SEQUENCE LISTING (90 sequences)

SEQ ID NO: 1 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 307 aa, Homo sapiens
ECGHISVSAPIVHLGDPITASCIIKQNCSHLDPEPQILWRLGAELQPGGRQQRLSDGTQESIITLPHLNHTQAFLSCSLNWGNSLQILDQVELRAGYPPAIPHNLSCLMNLTTSSLICQWEPGPETHLPTSFTLKSFKSRGNCQTQGDSILDCVPKDGQSHCSIPRKHLLLYQNMGIWVQAENALGTSMSPQLCLDPMDVVKLEPPMLRTMDPSPEAAPPQAGCLQLSWEPWQPGLHINQKCELRHKPQRGEASWALVGPLPLEALQYELCGLLPATAYTLQIRCIRWPLPGHWSDWSPSLELRTTE

SEQ ID NO: 3 - protein, 627 aa, Homo sapiens
MARLGNCSLTWAALIILLLPGSLEECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELH

SEQ ID NO: 4 - protein, 123 aa, Homo sapiens
AIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSIS

SEQ ID NO: 5 - protein, 205 aa, Homo sapiens
ASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 627 aa, Homo sapiens; identical to SEQ ID NO: 3

SEQ ID NO: 7 - protein, 328 aa, Homo sapiens
AIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 8 - protein, 627 aa, Homo sapiens; identical to SEQ ID NO: 3

SEQ ID NO: 9 - protein, 107 aa, Homo sapiens
VVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 10 - skipped

SEQ ID NO: 11 - protein, 24 aa, Homo sapiens
MARLGNCSLTWAALIILLLPGSLE

SEQ ID NO: 12 - protein, 22 aa, Homo sapiens
MLLLVTSLLLCELPHPAFLLIP

SEQ ID NO: 13 - DNA, 72 bp, Homo sapiens
atggcaaggctgggaaactgcagcctgacttgggctgccctgatcatcctgctgctccccggaagtctggag

SEQ ID NO: 14 - DNA, 66 bp, Mus musculus
atgctgctgctagtgacctccctgctgctctgtgagctgcctcacccggcgttcctgctgattcct

SEQ ID NO: 15 - protein, 603 aa, Homo sapiens
ECGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPEGSELH

SEQ ID NO: 16 - DNA, 1809 bp, Homo sapiens
gagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacggatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgtcccactccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcctcctcccgtggctttgtcctccatggcctggagcccgccagtctgtatcacatccacctcatggctgccagccaggctggggccaccaacagtacagtcctcaccctgatgaccttgaccccagaggggtcggagctacac

SEQ ID NO: 17 - DNA, 1809 bp, Artificial Sequence
  Description of Artificial Sequence: Synthetic polynucleotide
gagtgcgggcacatcagtgtctcagcccccatcgtccacctgggggatcccatcacagcctcctgcatcatcaagcagaactgcagccatctggacccggagccacagattctgtggagactgggagcagagcttcagcccgggggcaggcagcagcgtctgtctgatgggacccaggaatctatcatcaccctgccccacctcaaccacactcaggcctttctctcctgctgcctgaactggggcaacagcctgcagatcctggaccaggttgagctgcgcgcaggctaccctccagccataccccacaacctctcctgcctcatgaacctcacaaccagcagcctcatctgccagtgggagccaggacctgagacccacctacccaccagcttcactctgaagagtttcaagagccggggcaactgtcagacccaaggggactccatcctggactgcgtgcccaaggacgggcagagccactgctgcatcccacgcaaacacctgctgttgtaccagaatatgggcatctgggtgcaggcagagaatgcgctggggaccagcatgtccccacaactgtgtcttgatcccatggatgttgtgaaactggagccccccatgctgcggaccatggaccccagccctgaagcggcccctccccaggcaggctgcctacagctgtgctgggagccatggcagccaggcctgcacataaatcagaagtgtgagctgcgccacaagccgcagcgtggagaagccagctgggcactggtgggccccctccccttggaggcccttcagtatgagctctgcgggctcctcccagccacggcctacaccctgcagatacgctgcatccgctggcccctgcctggccactggagcgactggagccccagcctggagctgagaactaccgaacgggcccccactgtcagactggacacatggtggcggcagaggcagctggaccccaggacagtgcagctgttctggaagccagtgcccctggaggaagacagcggacgcatccaaggttatgtggtttcttggagaccctcaggccaggctggggccatcctgcccctctgcaacaccacagagctcagctgcaccttccacctgccttcagaagcccaggaggtggcccttgtggcctataactcagccgggacctctcgccccaccccggtggtcttctcagaaagcagaggcccagctctgaccagactccatgccatggcccgagaccctcacagcctctgggtaggctgggagccccccaatccatggcctcagggctatgtgattgagtggggcctgggcccccccagcgcgagcaatagcaacaagacctggaggatggaacagaatgggagagccacggggtttctgctgaaggagaacatcaggccctttcagctctatgagatcatcgtgactcccttgtaccaggacaccatgggaccctcccagcatgtctatgcctactctcaagaaatggctccctcccatgccccagagctgcatctaaagcacattggcaagacctgggcacagctggagtgggtgcctgagccccctgagctggggaagagcccccttacccactacaccatcttctggaccaacgctcagaaccagtccttctccgccatcctgaatgcatcctcccgtggctttgtcctccatggcctggagcccgccagtctgtatcacatccacctcatggctgccagccaggctggggccaccaacagtacagtcctcaccctgatgaccttgaccccagaggggtcggagctacac

SEQ ID NO: 18 - protein, 23 aa, Homo sapiens
IILGLFGLLLLLTCLCGTAWLCC

SEQ ID NO: 19 - protein, 22 aa, Homo sapiens
AIVVPVCLAFLLTTLLGVLFCF

SEQ ID NO: 20 - protein, 25 aa, Homo sapiens
IPWLGHLLVGLSGAFGFIILVYLLI

SEQ ID NO: 21 - protein, 21 aa, Homo sapiens
VVISVGSMGLIISLLCVYFWL

SEQ ID NO: 22 - DNA, 69 bp, Homo sapiens
atcatcctgggcctgttcggcctcctgctgttgctcacctgcctctgtggaactgcctggctctgttgc

SEQ ID NO: 23 - DNA, 66 bp, Homo sapiens
gccatagtcgtgcctgtttgcttagcattcctattgacaactcttctgggagtgctgttctgcttt

SEQ ID NO: 24 - DNA, 75 bp, Homo sapiens
attccgtggctcggccacctcctcgtgggtctcagcggggcttttggcttcatcatcttagtgtacttgctgatc

SEQ ID NO: 25 - DNA, 63 bp, Homo sapiens
gtggttatctctgttggctccatgggattgattatcagccttctctgtgtgtatttctggctg

SEQ ID NO: 26 - protein, 286 aa, Homo sapiens
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 27 - protein, 86 aa, Homo sapiens
ERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 28 - protein, 101 aa, Homo sapiens
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSIS

SEQ ID NO: 29 - protein, 205 aa, Homo sapiens; identical to SEQ ID NO: 5

SEQ ID NO: 30 - protein, 62 aa, Homo sapiens
SPNRKNPLWPSVPDPAHSSLGSWVPTIMEEDAFQLPGLGTPPITKLTVLEEDEKKPVPWESH

SEQ ID NO: 31 - protein, 202 aa, Homo sapiens
SSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 32 - protein, 67 aa, Homo sapiens
SPNRKNPLWPSVPDPAHSSLGSWVPTIMEEDAFQLPGLGTPPITKLTVLEEDEKKPVPWESHNSSET

SEQ ID NO: 33 - protein, 72 aa, Homo sapiens
AGDLPTHDGYLPSNIDDLPSHEAPLADSLEELEPQHISLSVFPSSSLHPLTFSCGDKLTLDQLKMRCDSLML

SEQ ID NO: 34 - protein, 67 aa, Homo sapiens; identical to SEQ ID NO: 32

SEQ ID NO: 35 - protein, 30 aa, Homo sapiens
SPGDEGPPRSYLRQWVVIPPPLSSPGPQAS

SEQ ID NO: 36 - protein, 63 aa, Homo sapiens
SPNRKNPLWPSVPDPAHSSLGSWVPTIMEEDAFQLPGLGTPPITKLTVLEEDEKKPVPWESHN

SEQ ID NO: 37 - protein, 183 aa, Homo sapiens
QNSGGSAYSEERDRPYGLVSIDTVTVLDAEGPCTWPCSCEDDGYPALDLDAGLEPSPGLEDPLLDAGTTVLSCGCVSAGSPGLGGPLGSLLDRLKPPLADGEDWAGGLPWGGRSPGGVSESEAGSPLAGLDMDTFDSGFVGSDCSSPVECDFTSPGDEGPPRSYLRQWVVIPPPLSSPGPQAS

SEQ ID NO: 38 - protein, 93 aa, Homo sapiens
SPNRKNPLWPSVPDPAHSSLGSWVPTIMEEDAFQLPGLGTPPITKLTVLEEDEKKPVPWESHNSSETCGLPTLVQTYVLQGDPRAVSTQPQSQ

SEQ ID NO: 39 - protein, 202 aa, Homo sapiens; identical to SEQ ID NO: 31

SEQ ID NO: 40 - protein, 62 aa, Homo sapiens; identical to SEQ ID NO: 30

SEQ ID NO: 41 - protein, 43 aa, Homo sapiens
SSNHSLTSCFTNQGYFFFHLPDALEIEACQVYFTYDPYSEEDP

SEQ ID NO: 42 - protein, 20 aa, Homo sapiens
AGDLPTHDGYLPSNIDDLPS

SEQ ID NO: 43 - protein, 116 aa, Homo sapiens
GGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 44 - protein, 93 aa, Homo sapiens; identical to SEQ ID NO: 38

SEQ ID NO: 45 - protein, 43 aa, Homo sapiens; identical to SEQ ID NO: 41

SEQ ID NO: 46 - protein, 20 aa, Homo sapiens
AGDLPTHDGYLPSNIDDLPS

SEQ ID NO: 47 - protein, 116 aa, Homo sapiens; identical to SEQ ID NO: 43

SEQ ID NO: 48 - protein, 138 aa, Homo sapiens
NKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSR

SEQ ID NO: 49 - protein, 205 aa, Homo sapiens; identical to SEQ ID NO: 5

SEQ ID NO: 50 - protein, 62 aa, Homo sapiens; identical to SEQ ID NO: 30

SEQ ID NO: 51 - protein, 67 aa, Homo sapiens
SGKNGPHVYQDLLLSLGTTNSTLPPPFSLQSGILTLNPVAQGQPILTSLGSNQEEAYVTMSSFYQNQ

SEQ ID NO: 52 - protein, 93 aa, Homo sapiens; identical to SEQ ID NO: 38

SEQ ID NO: 53 - protein, 77 aa, Homo sapiens
SSSRSLDCRESGKNGPHVYQDLLLSLGTTNSTLPPPFSLQSGILTLNPVAQGQPILTSLGSNQEEAYVTMSSFYQNQ

SEQ ID NO: 54 - DNA, 861 bp, Homo sapiens
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctcctgcagcaggacaaggtgcctgagcccgcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 55 - DNA, 261 bp, Homo sapiens
gaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctga

SEQ ID NO: 56 - DNA, 303 bp, Homo sapiens
aataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgtatgtcatcttctaggccaagcatttct

SEQ ID NO: 57 - DNA, 618 bp, Homo sapiens
gcatccttaagcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 58 - DNA, 186 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccat

SEQ ID NO: 59 - DNA, 609 bp, Homo sapiens
agcagcaaccactcgctgaccagctgcttcaccaaccagggttacttcttcttccacctcccggatgccttggagatagaggcctgccaggtgtactttacttacgacccctactcagaggaagaccctgatgagggtgtggccggggcacccacagggtcttccccccaacccctgcagcctctgtcaggggaggacgacgcctactgcaccttcccctccagggatgacctgctgctcttctcccccagtctcctcggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 60 - DNA, 201 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacc

SEQ ID NO: 61 - DNA, 219 bp, Homo sapiens
gcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctctga

SEQ ID NO: 62 - DNA, 201 bp, Homo sapiens; identical to SEQ ID NO: 60

SEQ ID NO: 63 - DNA, 93 bp, Artificial Sequence
  Description of Artificial Sequence: Synthetic oligonucleotide
agccctggggacgaaggacccccccggagctacctccgccagtgggtggtcattcctccgccactttcgagccctggaccccaggccagctaa

SEQ ID NO: 64 - DNA, 189 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataac

SEQ ID NO: 65 - DNA, 549 bp, Artificial Sequence
  Description of Artificial Sequence: Synthetic polynucleotide
cagaactcggggggctcagcttacagtgaggagagggatcggccatacggcctggtgtccattgacacagtgactgtgctagatgcagaggggccatgcacctggccctgcagctgtgaggatgacggctacccagccctggacctggatgctggcctggagcccagcccaggcctagaggacccactcttggatgcagggaccacagtcctgtcctgtggctgtgtctcagctggcagccctgggctaggagggcccctgggaagcctcctggacagactaaagccaccccttgcagatggggaggactgggctgggggactgccctggggtggccggtcacctggaggggtctcagagagtgaggcgggctcacccctggccggcctggatatggacacgtttgacagtggctttgtgggctctgactgcagcagccctgtggagtgtgacttcaccagccctggggacgaaggacccccccggagctacctccgccagtgggtggtcattcctccgccactttcgagccctggaccccaggccagc

SEQ ID NO: 66 - DNA, 279 bp, Homo sapiens
agccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagacctgtggcctccccactctggtccagacctatgtgctccagggggacccaagagcagtttccacccagccccaatcccag

SEQ ID NO: 67 - DNA, 609 bp, Homo sapiens; identical to SEQ ID NO: 59

SEQ ID NO: 68 - DNA, 186 bp, Homo sapiens; identical to SEQ ID NO: 58

SEQ ID NO: 69 - DNA, 129 bp, Homo sapiens
agcagcaaccactcgctgaccagctgcttcaccaaccagggttacttcttcttccacctcccggatgccttggagatagaggcctgccaggtgtactttacttacgacccctactcagaggaagaccct

SEQ ID NO: 70 - DNA, 60 bp, Homo sapiens
gcaggtgaccttcccacccatgatggctacttaccctccaacatagatgacctcccctca

SEQ ID NO: 71 - DNA, 351 bp, Homo sapiens
ggtggccccagccccccaagcactgcccctgggggcagtggggccggtgaagagaggatgcccccttctttgcaagaaagagtccccagagactgggacccccagcccctggggcctcccaccccaggagtcccagacctggtggattttcagccaccccctgagctggtgctgcgagaggctggggaggaggtccctgacgctggccccagggagggagtcagtttcccctggtccaggcctcctgggcagggggagttcagggcccttaatgctcgcctgcccctgaacactgatgcctacttgtccctccaagaactccagggtcaggacccaactcacttggtgtag

SEQ ID NO: 72 - DNA, 279 bp, Homo sapiens; identical to SEQ ID NO: 66

SEQ ID NO: 73 - DNA, 129 bp, Homo sapiens; identical to SEQ ID NO: 69

SEQ ID NO: 74 - DNA, 60 bp, Homo sapiens; identical to SEQ ID NO: 70

SEQ ID NO: 75 - DNA, 351 bp, Homo sapiens; identical to SEQ ID NO: 71

SEQ ID NO: 76 - DNA, 414 bp, Artificial Sequence
  Description of Artificial Sequence: Synthetic polynucleotide
aataagcgagacctaattaaaaaacacatctggcctaatgttccagatccttcaaagagtcatattgcccagtggtcacctcacactcctccaaggcacaatttcaattcaaaggatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgtatgtcatcttctaggccaagcatttctagcagtgatgaaaatgaatcttcacaaaacacttcgagcactgtccagtattctaccgtggtacacagtggctacagacaccaagttccgtcagtccaagtcttctcaaga

SEQ ID NO: 77 - DNA, 618 bp, Artificial Sequence; identical to SEQ ID NO: 57
  Description of Artificial Sequence: Synthetic polynucleotide

SEQ ID NO: 78 - DNA, 186 bp, Homo sapiens; identical to SEQ ID NO: 58

SEQ ID NO: 79 - DNA, 204 bp, Homo sapiens
agtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 80 - DNA, 279 bp, Homo sapiens; identical to SEQ ID NO: 66

SEQ ID NO: 81 - DNA, 234 bp, Homo sapiens
tcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 82 - protein, 40 aa, Artificial Sequence
  Description of Artificial Sequence: Synthetic polypeptide
AAAENLYFQGSAWSHPQFEKGGGSGGGSGGSAWSHPQFEK

SEQ ID NO: 83 - protein, 20 aa, Artificial Sequence
  Description of Artificial Sequence: Synthetic polypeptide
HHHHHHSSGRENLYFQGSMG

SEQ ID NO: 84 - protein, 9 aa, Homo sapiens
IWPNVPDPS

SEQ ID NO: 85 - protein, 9 aa, Homo sapiens
LKCNTPDPS

SEQ ID NO: 86 - protein, 9 aa, Homo sapiens
KIWAVPSPE

SEQ ID NO: 87 - protein, 9 aa, Homo sapiens
CSREIPDPA

SEQ ID NO: 88 - protein, 9 aa, Homo sapiens
VWPSLPDHK

SEQ ID NO: 89 - protein, 6 aa, Artificial Sequence
  Description of Artificial Sequence: Synthetic 6xHis tag
HHHHHH

SEQ ID NO: 90 - protein, 175 aa, Artificial Sequence
  Description of Artificial Sequence: Synthetic polypeptide
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

(19) United States
(12) Patent Application Publication
(10) Pub. No.: US 2024/0115603 A1
(43) Pub. Date: Apr. 11, 2024

(54) Modified Extracellular Domain of Granulocyte Colony-Stimulating Factor Receptor (G-CSFG) and Cytokines Binding Same

(71) Applicants: Zymeworks Inc., Vancouver (CA); Provincial Health Services Authority, Vancouver (CA); UVic Industry Partnerships Inc., Victoria (CA)

(72) Inventors: Gesa Volkers, Vancouver (CA); Megan Fuller, Vancouver (CA); Martin J. Boulanger, Victoria (CA); Surjit Bhimarao Dixit, Vancouver (CA); Brad Nelson, Vancouver (CA); Mario Sanches, Vancouver (CA); Bianca Loveless, Victoria (CA)

(21) Appl. No.: 17/767,682

(22) PCT Filed: Oct. 8, 2020
(86) PCT No.: PCT/CA2020/051352

§ 371 Date: Apr. 8, 2022

Related U.S. Application Data
(60) Provisional application No. 62/912,318, filed on Oct. 8, 2019.

(51) Int. Cl. A61K 35/17; A61K 39/00; A61K 45/06; A61P 37/04; C07K 14/715; C12N 5/0783

(52) U.S. Cl. CPC A61K 35/17; A61K 39/4611; A61K 39/4613; A61K 39/464418; A61K 45/06; A61P 37/04; C07K 14/7153; C12N 5/0636; C12N 5/0646

(57) ABSTRACT

Described herein are methods and compositions for selective activation of cells using variant cytokine receptor and cytokine pairs, wherein the cytokine receptors comprise an extracellular domain (ECD) of granulocyte-colony stimulating factor receptor (G-CSFR). In certain embodiments, the methods and compositions described herein are useful for exclusive activation of cells for adoptive cell transfer therapy. Thus, included herein are methods of producing cells expressing variant receptors that are selectively activated by a cytokine that does not bind its native receptor. Also disclosed herein are methods of treating a subject in need thereof, comprising administering to the subject cells expressing an variant receptor comprising an extracellular domain of G-CSFR and co-administering a variant cytokine that activates the variant receptor.

### CROSS REFERENCE TO RELATED APPLICATIONS

The application claims priority to U.S. Provisional Application No. 62/912,318, filed Oct. 8, 2019, which is hereby incorporated by reference in its entirety.

### SEQUENCE LISTING

The instant application contains a Sequence Listing which has been submitted via EFS-Web and is hereby incorporated by reference in its entirety. Said ASCII copy, created on Oct. 7, 2020, is named IKE-068WO_US_SL.txt, and is 85,109 bytes in size.

### BACKGROUND OF THE INVENTION

### Field of the Invention

The invention relates to methods and compositions for selective activation of cells using variant cytokine receptor and cytokine pairs, wherein the cytokine receptors comprise a variant extracellular domain (ECD) of granulocyte-colony stimulating factor receptor (G-CSFR). Also disclosed herein are methods of treating a subject by adoptive cell transfer, comprising administering to the subject cells expressing variant receptors and administering variant cytokines to send signals to the cells expressing variant receptors. Included with this disclosure are nucleic acids, expression vectors and kits for producing cells expressing variant cytokines and receptors, and kits which also provide cytokines for binding variant receptors.

### Description of the Related Art

The past two decades have brought much progress in the treatment of cancer by adoptive cell therapy (ACT). ACT with naturally occurring tumor-infiltrating T cells (TIL) is now reproducibly yielding >50% objective clinical response rates in advanced melanoma. ACT with T cells engineered to recognize B-lineage leukemias (using CD19-directed chimeric antigen receptors, or CD19 CARs) is yielding up to 90% complete response rates, with the majority of patients achieving durable responses. Motivated by these remarkable results, several companies are commercializing the TIL and CD19 CAR T cell approaches.

The engraftment, expansion and persistence of T cells for ACT are important determinants of clinical safety and efficacy. This is often addressed by administration of systemic IL-2 after ACT transfer, as well as expanding the T cells outside the body with IL-2 prior to transfer. In addition to the intended immune stimulatory effects, systemic IL-2 treatment can also lead to severe toxicities such as vascular leakage syndrome that need to be tightly controlled for patient safety. To manage these risks, patients typically need to be hospitalized for 2-3 weeks and have access to an ICU as a precautionary measure. Furthermore, IL-2 induces the proliferation of both effector and regulatory (inhibitory) T cells (5); therefore, giving a patient IL-2 is analogous to pressing the gas and brake pedals at the same time. CAR T cells bring the converse problem in that T-cell expansion and persistence can exceed safe levels. Moreover, they universally eradicate normal B cells (which also express CD19), leaving patients partly immune-deficient for life. Ideally, one would like to have precise control over the number of tumor-reactive T cells after adoptive transfer, including the ability to eliminate transferred cells once the cancer has been eradicated. Other cell-based therapies, such as stem cell therapies, would also benefit from improved control over the expansion, differentiation, and persistence of infused cells.

Human G-CSF is an approved therapeutic (Neupogen®, Filgrastim) used to treat Neutropenia in cancer patients. G-CSF is a four-helix bundle (Hill, C P et al. Proc Natl Acad Sci USA. 1993 Jun. 1; 90(11):5167-71), and the structure of G-CSF in complex with its receptor G-CSFR is well characterized (Tamada, T et al. Proc Natl Acad Sci USA. 2006 Feb. 28; 103(9):3135-40). The G-CSF:G-CSFR complex is a 2:2 heterodimer. G-CSF has two binding interfaces with G-CSFR. One interface is referred to as site II; it is the larger interface between G-CSF and the Cytokine Receptor Homologous (CRH) domain of G-CSFR. The second interface is referred to as site III; it is the smaller interface between G-CSF and the N-terminal Ig-like domain of G-CSFR.

### SUMMARY OF THE INVENTION

In certain embodiments, disclosed herein is a receptor, comprising a variant extracellular domain (ECD) of Granulocyte Colony-Stimulating Factor Receptor (G-CSFR), wherein the variant ECD of G-CSFR comprises at least one mutation in a site II interface region, at least one mutation in a site III interface region, or combinations thereof. In certain aspects, at least one mutation in the site II interface region is located at an amino acid position of the G-CSFR ECD selected from the group consisting of amino acid position 141, 167, 168, 171, 172, 173, 174, 197, 199, 200, 202 and 288 of SEQ ID NO. 2. In certain aspects, at least one mutation in the site II interface region is selected from the group of mutations of the G-CSFR ECD consisting of R141E, R167D, K168D, K168E, L171E, L172E, Y173K, Q174E, D197K, D197R, M199D, D200K, D200R, V202D, R288D, and R288E. In certain aspects, at least one mutation in the site III interface region is selected from the group of mutations of the G-CSFR ECD selected from the group consisting of amino acid position 30, 41, 73, 75, 79, 86, 87, 88, 89, 91, and 93 of SEQ ID NO. 2. In certain aspects, at least one mutation in the site III interface region is selected from the group of mutations of the G-CSFR ECD consisting of S30D, R41E, Q73W, F75KF, S79D, L86D, Q87D, I88E, L89A, Q91D, Q91K, and E93K. In certain aspects, the G-CSFR ECD comprises a combination of mutations of a design number in Table 6; wherein the mutations correspond to an amino acid position of SEQ ID NO. 2. In certain aspects, the G-CSFR ECD comprises the mutations: R41E, R141E, and R167D.

In certain aspects, the receptor disclosed herein is a chimeric receptor. In certain aspects, the receptor is expressed on a cell. In certain aspects, the receptor is expressed on an immune cell. In certain aspects, the immune cell is optionally a T cell, and, optionally, a NK cell, and, optionally, a NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell.

In certain aspects, the activation of the receptor by a variant G-CSF causes a cellular response selected from the group consisting of proliferation, viability and enhanced activity of a cell expressing the receptor.

In certain embodiments, disclosed herein is a nucleic acid encoding any of the receptors disclosed herein. In certain aspects, described herein is an expression vector comprising the nucleic acid. In certain embodiments, described herein is a cell engineered to express receptors disclosed herein. In certain aspects, the cell is an immune cell. In certain embodiments, the immune cell is optionally a T cell, and, optionally, a NK cell, and, optionally, a NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In certain embodiments, disclosed herein is a variant Granulocyte Colony-Stimulating Factor (G-CSF), wherein the variant G-CSF comprises at least one mutation in a site II interface region, at least one mutation in a site III interface region, or combinations thereof. In certain aspects, at least one mutation in the site II interface region of the variant G-CSF is located at an amino acid position selected from the group consisting of amino acid position 12, 16, 19, 20, 104, 108, 109, 112, 115, 116, 118, 119, 122 and 123 of SEQ ID NO. 1. In certain aspects, at least one mutation in the site II interface region of the variant G-CSF is selected from the group of mutations consisting of: S12E, S12K, S12R, K16D, L18F, E19K, Q20E, D104K, D104R, L108K, L108R, D109R, D112R, D112K, T115E, T115K, T116D, Q119E, Q119R, E122K, E122R, and E123R. In certain aspects, at least one mutation in the site III interface region of the variant G-CSF is selected from the group of mutations selected from the group consisting of amino acid position 38, 39, 40, 41, 46, 47, 48, 49, and 147 of SEQ ID NO. 1. In certain aspects, at least one mutation in the site III interface region of the variant G-CSF is selected from the group of mutations consisting of: T38R, Y39E, K40D, K40F, L41D, L41E, L41K, E46R, L47D, V48K, V48R, L49K, and R147E. In certain aspects, the variant G-CSF comprises a combination of mutations of a design number in Table 6; wherein the mutations correspond to an amino acid position of SEQ ID NO. 1. In certain aspects, the variant G-CSF comprises the mutations: E46R, L108K and D112R. In certain aspects, the variant G-CSF binds selectively to a receptor disclosed herein. In certain aspects, the receptor is expressed on a cell. In certain aspects, the cell is an immune cell. In certain aspects, the immune cell is optionally a T cell, and, optionally, a NK cell, and, optionally, a NKT cell, and, optionally, a B cell, and, optionally, a plasma cell, and, optionally, a macrophage, and, optionally, a dendritic cell, and, optionally, the cell is a stem cell, and, optionally, the cell is a primary cell, and, optionally, the cell is a human cell. In certain aspects, the selective binding of the variant G-CSF to the receptor causes a cellular response selected from the group consisting of proliferation, viability and enhanced activity of the immune cell.

In certain embodiments, disclosed herein is a nucleic acid encoding a variant G-CSF. In certain aspects, disclosed herein is an expression vector comprising the nucleic acid. In certain embodiments, disclosed herein is a cell engineered to express a variant G-CSF. In certain aspects, the cell is an immune cell.

In certain embodiments, disclosed herein is a system for selective activation of a receptor expressed on a cell surface, the system comprising a receptor and a variant G-CSF wherein the receptor comprises at least one mutation in a site II interface region, a site III interface region, or combinations thereof, and the variant G-CSF comprises at least one mutation in an amino acid sequence of G-CSF that binds the site II interface region of the receptor, the site III interface region of the receptor, or combinations thereof; and wherein the variant G-CSF binds the receptor preferentially over a wild type G-CSFR ECD, and the receptor binds the variant G-CSF preferentially over a wild type G-CSF. In certain aspects, the receptor and the variant G-CSF comprise a combination of mutations of a site II interface of a design number of Table 2; wherein the receptor mutations correspond to an amino acid position of SEQ ID NO. 2 and the variant G-CSF mutations correspond to an amino acid position of SEQ ID NO. 1. In certain aspects, the receptor and the variant G-CSF comprise a combination of mutations of a site III interface of a design number of Table 4; wherein the receptor mutations correspond to an amino acid position of SEQ ID NO. 2 and the variant G-CSF mutations correspond to an amino acid position of SEQ ID NO. 1. In certain aspects, the receptor and the variant G-CSF comprise a combination of mutations of a site II interface, and a site III interface of a design number of Table 6; wherein the receptor mutations correspond to an amino acid position of SEQ ID NO. 2 and the variant G-CSF mutations correspond to an amino acid position of SEQ ID NO. 1. In certain aspects, the combination of mutations comprises the mutations of design number 106; wherein the variant G-CSF comprises the E46R and D104K mutations corresponding to an amino acid position of SEQ ID NO. 1; and wherein the receptor comprises the R41E and K168D mutations corresponding to an amino acid position of SEQ ID NO. 2. In certain aspects, the combination of mutations comprises the mutations of design number 117; wherein the variant G-CSF comprises the E46R, E122R, and E123R mutations corresponding to an amino acid position of SEQ ID NO. 1; and wherein the receptor comprises the R41E and R141E mutations corresponding to an amino acid position of SEQ ID NO. 2. In certain aspects, the combination of mutations comprises the mutations of design number 130; wherein the variant G-CSF comprises the E46R, L108K, and D112R mutations corresponding to an amino acid position of SEQ ID NO. 1; and wherein the receptor comprises the R41E and R167D mutations corresponding to an amino acid position of SEQ ID NO. 2. In certain aspects, the combination of mutations comprises the mutations of design number 134; wherein the variant G-CSF comprises the E46R, L108K, D112R, E122R, and E123R mutations corresponding to an amino acid position of SEQ ID NO. 1; and wherein the receptor comprises the R41E, R141E, and R167D mutations corresponding to an amino acid position of SEQ ID NO. 2. In certain aspects, the combination of mutations comprises the mutations of design number 135; wherein the variant G-CSF comprises the E46R, T115K, E122R, and E123R mutations corresponding to an amino acid position of SEQ ID NO. 1; and wherein the receptor comprises the R41E, R141E, L171E, and Q174E mutations corresponding to an amino acid position of SEQ ID NO. 2. In certain aspects, the combination of mutations comprises the mutations of design number 137; wherein the variant G-CSF comprises the E46R, L108K, and D112R mutations corresponding to an amino acid position of SEQ ID NO. 1; and wherein the receptor comprises the R41E, R141E, and R167D mutations corresponding to an amino acid position of SEQ ID NO. 2. In certain aspects, the combination of mutations comprises the mutations of design number 300; wherein the variant G-CSF comprises the K40D, L41D, L108K, and D112R mutations corresponding to an amino acid position of SEQ ID NO. 1; and wherein the receptor comprises the F75K, Q91K and R167D mutations corresponding to an amino acid position of SEQ ID NO. 2. In certain aspects, the combination of mutations comprises the mutations of design number 301; wherein the variant G-CSF comprises the T38R, E46R, L108K, and D112R mutations corresponding to an amino acid position of SEQ ID NO. 1; and wherein the receptor comprises the R41E, Q73E, and R167D mutations corresponding to an amino acid position of SEQ ID NO. 2. In certain aspects, the combination of mutations comprises the mutations of design number 302; wherein the variant G-CSF comprises the E46R, L108K, and D112R mutations corresponding to an amino acid position of SEQ ID NO. 1; and wherein the receptor comprises the R41E, L86D, and R167D mutations corresponding to an amino acid position of SEQ ID NO. 2. In certain aspects, the combination of mutations comprises the mutations of design number 303; wherein the variant G-CSF comprises the L108K, D112R, and R147E mutations corresponding to an amino acid position of SEQ ID NO. 1; and wherein the receptor comprises the E93K and R167D mutations corresponding to an amino acid position of SEQ ID NO. 2. In certain aspects, the combination of mutations comprises the mutations of design number 304; wherein the variant G-CSF comprises the E46R, L108K, D112R, and R147E mutations corresponding to an amino acid position of SEQ ID NO. 1; and wherein the receptor comprises the R41E, E93K, and R167D mutations corresponding to an amino acid position of SEQ ID NO. 2. In certain aspects, the combination of comprises the mutations of design number 305; wherein the variant G-CSF comprises the E19K, E46R, L108K, and D112R mutations corresponding to an amino acid position of SEQ ID NO. 1; and wherein the receptor comprises the R41E, R167D, and R288E mutations corresponding to an amino acid position of SEQ ID NO. 2. In certain aspects, the combination of mutations comprises the mutations of design number 307; wherein the variant G-CSF comprises the S12E, K16D, E19K, and E46R mutations corresponding to an amino acid position of SEQ ID NO. 1; and wherein the receptor comprises the R41E, D197K, D200K and R288E mutations corresponding to an amino acid position of SEQ ID NO. 2. In certain aspects, the combination of mutations comprises the mutations of design number 308; wherein the variant G-CSF comprises the E19R, E46R, D112K mutations corresponding to an amino acid position of SEQ ID NO. 1; and wherein the receptor comprises the R41E, R167D, V202D and R288E mutations corresponding to an amino acid position of SEQ ID NO. 2. In certain aspects, the combination of mutations comprises the mutations of design number 400; wherein the variant G-CSF comprises the E19K, E46R, D109R, and D112R mutations corresponding to an amino acid position of SEQ ID NO. 1; and wherein the receptor comprises the R41E, R167D, M199D and R288D mutations corresponding to an amino acid position of SEQ ID NO. 2. In certain aspects, the combination of mutations comprises the mutations of design number 401; wherein the variant G-CSF comprises the E19K, E46R, L108K, and D112R mutations corresponding to an amino acid position of SEQ ID NO. 1; and wherein the receptor comprises the R41E, R167D, and R288D mutations corresponding to an amino acid position of SEQ ID NO. 2. In certain aspects, the combination of mutations comprises the mutations of design number 402; wherein the variant G-CSF comprises the E19K, E46R, D112K, and T115K mutations corresponding to an amino acid position of SEQ ID NO. 1; and wherein the receptor comprises the R41E, R167E, Q174E and R288E mutations corresponding to an amino acid position of SEQ ID NO. 2. In certain aspects, the combination of mutations comprises the mutations of design number 403; wherein the variant G-CSF comprises the E19R, E46R, and D112K, mutations corresponding to an amino acid position of SEQ ID NO. 1; and wherein the receptor comprises the R41E, R167D, and R288E mutations corresponding to an amino acid position of SEQ ID NO. 2.

In certain embodiments, disclosed herein is a method of selective activation of a receptor expressed on the surface of a cell, comprising contacting a receptor with a variant G-CSF. In certain aspects, the receptor is expressed on a cell. In certain aspects, the receptor is expressed on an immune cell. In certain aspects, the receptor is expressed on a T cell or NK cell. In certain aspects, the selective activation of the immune cell causes a cellular response selected from the group consisting of proliferation, viability and enhanced activity of the immune cell.

In certain embodiments, disclosed herein is a method of producing a cell expressing a receptor disclosed herein, comprising introducing to the cells the nucleic acid or the expression vector disclosed herein.

In certain embodiments, disclosed herein is a method of treating a subject in need thereof, comprising infusing into the subject a cell (e.g., an immune cell) disclosed herein. In certain aspects, the method further comprises administering a variant G-CSF to the subject.

In certain embodiments, disclosed herein is a kit for producing a system for selective activation of a receptor expressed on a cell surface, the kit comprising a nucleic acid or the expression vector disclosed herein; a variant G-CSF disclosed herein, and instructions for use.

In certain embodiments, described herein are chimeric receptors, comprising: (a) an extracellular domain (ECD) of a G-CSFR (Granulocyte-Colony Stimulating Factor Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor) and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the at least one signaling molecule binding site is selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain.

In certain aspects, described herein are chimeric receptors, comprising: an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

In certain embodiments, the activated chimeric receptor forms a homodimer, and, optionally, the activation of the chimeric receptor causes a cellular response selected from the group consisting of proliferation, viability and enhanced activity of a cell expressing the chimeric receptor, and, optionally, the chimeric receptor is activated upon contact with a G-CSF, and, optionally, the G-CSF is a wild-type G-CSF, and, optionally, the extracellular domain of the G-CSFR is a wild-type extracellular domain. In certain embodiments, the activated chimeric receptor forms a homodimer, and, optionally, the activation of the chimeric receptor causes a cellular response selected from the group consisting of proliferation, viability and enhanced activity of a cell expressing the chimeric receptor, and, optionally, the chimeric receptor is activated upon contact with a G-CSF, and, optionally, the G-CSF is a wild-type G-CSF, and, optionally, the extracellular domain of the G-CSFR is a wild-type extracellular domain.

In certain embodiments, the chimeric receptor is expressed in a cell, and, optionally, an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and, optionally, the cell is a primary cell, and, optionally, the cell is a human cell.

In certain embodiments, the ICD comprises: (a) at least a portion of an ICD of IL-2Rβ having an amino acid sequence of SEQ ID NO. 16, 19, 21, 29, 31, 33, 35, 37, or 39; or (b) at least a portion of an ICD of IL-7Rα having an amino acid sequence of SEQ ID NO. 41; or (c) at least a portion of an ICD of IL-21R having an amino acid sequence of SEQ ID NO. 25 or 27; or (d) at least a portion of an ICD of IL-12Rβ₂ having an amino acid sequence of SEQ ID NO. 23, 32 or 26; or (e) at least a portion of an ICD of G-CSFR having an amino acid sequence of SEQ ID NO. 20, 22, 24, 26, 28, 30, 34, 40 or 42; or (0 at least a portion of an ICD of gp130 having an amino acid sequence of SEQ ID NO. 18 or 38; or (g) at least a portion of an ICD of IL-7R having an amino acid sequence of SEQ ID NO. 43; or (h) at least a portion of an ICD of IL-2RG having an amino acid sequence of SEQ ID NO. 17.

In certain embodiments, the transmembrane domain comprises a sequence set forth by:

### (a) SEQ ID NO. 8; or (b) SEQ ID NO. 9; or (c) SEQ ID NO. 10; or (d) SEQ ID NO. 11.

In certain aspects, described herein is a nucleic acid encoding a chimeric receptor; wherein the chimeric receptor comprises: (a) an extracellular domain (ECD) of a G-CSFR (Granulocyte-Colony Stimulating Factor Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ; and, optionally, the transmembrane domain is a wild-type transmembrane domain. In certain embodiments, the ECD of the G-CSFR is encoded by nucleic acid sequence set forth in SEQ ID NO. 5 or 6. In certain embodiments, the nucleic acid comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

In certain embodiments, the present disclosure describes an expression vector comprising a nucleic acid encoding a chimeric receptor described herein. In certain embodiments, the vector is selected from the group consisting of: a retroviral vector, a lentiviral vector, an adenoviral vector and a plasmid.

In certain aspects, described herein is a nucleic acid encoding a chimeric receptor; wherein the chimeric receptor comprises: an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

In certain embodiments, the ECD of the G-CSFR is encoded by nucleic acid sequence set forth in SEQ ID NO. 5 or 6. In certain embodiments, the nucleic acid comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7Rα having an amino acid sequence of SEQ ID NO. 43; or
(h) a sequence encoding at least a portion of an ICD of IL-2Rγc having an amino acid sequence of SEQ ID NO. 17.

In certain aspects, described herein are expression vectors comprising a nucleic acid described herein. In certain embodiments, the vector is selected from the group consisting of: a retroviral vector, a lentiviral vector, an adenoviral vector and a plasmid.

In certain aspects, described herein is a cell comprising a nucleic acid encoding a chimeric receptor; wherein the chimeric receptor comprises: (a) an extracellular domain (ECD) of a G-CSFR (Granulocyte-Colony Stimulating Factor Receptor) operatively linked Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain; and, optionally,
the cell is an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain aspects, described herein is a cell comprising a nucleic acid encoding a chimeric receptor; wherein the chimeric receptor comprises: an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα; and, optionally,
the cell is an immune cell; and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain embodiments, the ECD of the G-CSFR is encoded by nucleic acid comprised in the cell has a sequence set forth in SEQ ID NO. 5 or 6. In certain embodiments, the nucleic acid comprised by the cell comprises:
(a) a sequence encoding at least a portion of an ICD of IL-2Rβ having an amino sequence of SEQ ID NO. 16, 19, 21, 29, 31, 33, 35, 37, or 39; or
(b) a sequence encoding at least a portion of an ICD of IL-7Rα having an amino acid sequence of SEQ ID NO. 41; or
(c) a sequence encoding at least a portion of an ICD of IL-21R having an amino acid sequence of SEQ ID NO. 25 or 27; or
(d) a sequence encoding at least a portion of an ICD of IL-12Rβ₂ having an amino acid sequence of SEQ ID NO. 23, 32 or 26; or
(e) a sequence encoding at least a portion of an ICD of G-CSFR having an amino acid sequence of SEQ ID NO. 20, 22, 24, 26, 28, 30, 34, 40 or 42; or
(f) a sequence encoding at least a portion of an ICD of gp130 having an amino acid sequence of SEQ ID NO. 18 or 38; or
(g) a sequence encoding at least a portion of an ICD of IL-7R having an amino acid sequence of SEQ ID NO. 43; or
(h) a sequence encoding at least a portion of an ICD of IL-2Rγc having an amino acid sequence of SEQ ID NO. 17.

In certain aspects, described herein is a cell comprising an expression vector described herein, and, optionally, the cell is an immune cell, and, optionally, a T cell or a NK cell. In certain aspects, described herein is a cell comprising the chimeric receptor of claim 1, and, optionally, the cell in an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain aspects, described herein is a cell comprising the chimeric receptor described herein, and, optionally, the cell in an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain aspects, described herein is a method of selective activation of a chimeric receptor expressed on the surface of a cell, comprising: contacting a chimeric receptor with a G-CSF that selectively activates the chimeric receptor; wherein the chimeric receptor comprises: (a) an extracellular domain (ECD) of a G-CSFR (Granulocyte-Colony Stimulating Factor Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor), and, optionally, the IL-2R is selected from the group consisting of IL-2Rβ and IL-2Rγc, and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor, and, optionally, the at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R, and, optionally, the ICD comprises a Box 1 region and a Box 2 region of a protein selected from the group consisting of G-CSFR and gp130; and, optionally, the chimeric receptor comprises a third domain comprising at least a portion of a transmembrane domain of a protein selected from the group consisting of: G-CSFR, gp130 (Glycoprotein 130), and IL-2Rβ, and, optionally, the transmembrane domain is a wild-type transmembrane domain.

In certain aspects, described herein is a method of selective activation of a chimeric receptor expressed on the surface of a cell, comprising: contacting a chimeric receptor with a G-CSF that selectively activates the chimeric receptor; wherein the chimeric receptor, comprises an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

In certain embodiments of the methods described herein, the activated chimeric receptor forms a homodimer, and, optionally, the activation of the chimeric receptor causes a cellular response selected from the group consisting of proliferation, viability and enhanced activity of a cell expressing the chimeric receptor; and, optionally, the chimeric receptor is activated upon contact with a G-CSF, and, optionally, the G-CSF is a wild-type G-CSF, and, optionally, the extracellular domain of the G-CSFR is a wild-type extracellular domain; wherein the chimeric receptor is expressed in a cell, and, optionally, an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell.

In certain embodiments of the methods described herein, the chimeric receptor comprises
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or
(c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17; and wherein the transmembrane domain comprises a sequence set forth by: (a) SEQ ID NO. 8; or
(b) SEQ ID NO. 9; or (c) SEQ ID NO. 10; or (d) SEQ ID NO. 11.

In certain aspects, described herein is a method of producing a chimeric receptor in an cell, comprising: introducing into the cell the nucleic acid of any one of claim 13-16, or 19-22 or the expression vector of any one of claims claim 17, 18, 23 or 24; and, optionally, the method comprises gene editing; and, optionally, the cell is an immune cell and, optionally, a T cell, and, optionally, a NK cell, and, optionally, a NKT cell, and, optionally, a B cell, and, optionally, a plasma cell, and optionally, a macrophage, and optionally, a dendritic cell, and optionally, the cell is a primary cell, and, optionally, the cell is a human cell.

In certain embodiments, described herein is a method of treating a subject in need thereof, comprising: infusing into the subject a cell expressing a chimeric receptor and administering a cytokine that binds the chimeric receptor; wherein the chimeric receptor comprises: (a) an extracellular domain (ECD) of a G-CSFR (Granulocyte-Colony Stimulating Factor Receptor) operatively linked to a second domain; the second domain comprising (b) at least a portion of an intracellular domain (ICD) of a multi-subunit cytokine receptor selected from the group consisting of: IL-2R (Interleukin-2 receptor), IL-7R (Interleukin-7 receptor), IL-12R (Interleukin-12 Receptor), and IL-21R (Interleukin-21 Receptor); and, optionally, the IL-2R is selected from the group consisting of IL-2Rβ and IL-2Rγc; and, optionally, the second domain comprises at least a portion of the C-terminal region of IL-2Rβ, IL-7Rα, IL-12Rβ₂ or IL-21R; wherein at least a portion of the ICD of the cytokine receptor comprises at least one signaling molecule binding site from an intracellular domain of a cytokine receptor; and, optionally, the ICD comprises at least one signaling molecule binding site selected from the group consisting of: a STAT3 binding site of G-CSFR; a STAT3 binding site of gp130; a SHP-2 binding site of gp130; a SHC binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT4 binding site of IL-12Rβ₂; a STAT5 binding site of IL-12Rβ₂; a STAT3 binding site of IL-12Rβ₂; a STAT5 binding site of IL-21R; a STAT3 binding site of IL-21R; and a STAT1 binding site of IL-21R; and, optionally, the ICD comprises a Box 1 region and a Box 2 region of a protein selected from the group consisting of G-CSFR and gp130; and, optionally, the chimeric receptor comprises comprising a third domain comprising at least a portion of a transmembrane domain of a protein selected from the group consisting of: G-CSFR, gp130 (Glycoprotein 130), and IL-2Rβ; and, optionally, the transmembrane domain is a wild-type transmembrane domain.

In certain aspects, described herein is a method of treating a subject in need thereof, comprising: infusing into the subject a cell expressing a chimeric receptor and administering a cytokine that binds the chimeric receptor; wherein the chimeric receptor comprises: an ECD of a G-CSFR operatively linked to a second domain; the second domain comprising:
(i)
(a) a transmembrane domain of gp130;
(b) a Box 1 and a Box 2 region of gp130; and
(c) a C-terminal region of IL-2Rβ; or
(ii)
(a) a transmembrane domain of G-CSFR;
(b) a Box 1 and a Box 2 region of G-CSFR; and
(c) a C-terminal region of IL-2Rβ; or
(iii)
(a) a transmembrane domain of G-CSFR;
(b) a Box 1 and a Box 2 region of G-CSFR; and
(c) a C-terminal region of IL-12Rβ₂; or
(iv)
(a) a transmembrane domain of G-CSFR;
(b) a Box 1 and a Box 2 region of G-CSFR; and
(c) a C-terminal region of IL-21R; or
(v)
(a) a transmembrane domain of IL-2Rβ+γc;
(b) a Box 1 and a Box 2 region of IL-2Rβ+γc; and
(c) a C-terminal region of IL-2Rβ+γc; or
(vi)
(a) a transmembrane domain of G-CSFR
(b) a Box 1 and a Box 2 region of G-CSFR; and
(c) a C-terminal region of IL-7Rα.

In certain embodiments of the method, the activated chimeric receptor forms a homodimer; and, optionally, the activation of the chimeric receptor causes a cellular response selected from the group consisting of proliferation, viability and enhanced activity of a cell expressing the chimeric receptor; and, optionally, the chimeric receptor is activated upon contact with a G-CSF; and, optionally, the G-CSF is a wild-type G-CSF; and, optionally, the extracellular domain of the G-CSFR is a wild-type extracellular domain; wherein the chimeric receptor is expressed in a cell; and, optionally, the cell is an immune cell, and, optionally, a T cell, and, optionally, a NK cell, and, optionally, a NKT cell, and, optionally, a B cell, and, optionally, a plasma cell, and optionally, a macrophage, and optionally, a dendritic cell, and, optionally, the cell is a stem cell, and optionally, the cell is a primary cell, and, optionally, the cell is a human cell. In certain embodiments, the chimeric receptor optionally comprises:
(a) at least a portion of an ICD of IL-2Rβ having an amino acid sequence of SEQ ID NO. 16, 19, 21, 29, 31, 33, 35, 37, or 39; or
(b) at least a portion of an ICD of IL-7Rα having an amino acid sequence of SEQ ID NO. 41; or
(c) at least a portion of an ICD of IL-21R having an amino acid sequence of SEQ ID NO. 25 or 27; or
(d) at least a portion of an ICD of IL-12Rβ₂ having an amino acid sequence of SEQ ID NO. 23, 32 or 26; or
(e) at least a portion of an ICD of G-CSFR having an amino acid sequence of SEQ ID NO. 20, 22, 24, 26, 28, 30, 34, 40 or 42; or
(f) at least a portion of an ICD of gp130 having an amino acid sequence of SEQ ID NO. 18 or 38; or
(g) at least a portion of an ICD of IL-7Rα having an amino acid sequence of SEQ ID NO. 43; or
(h) at least a portion of an ICD of IL-2Rγc having an amino acid sequence of SEQ ID NO. 17; and wherein the transmembrane domain comprises a sequence set forth by: (a) SEQ ID NO. 8; or (b) SEQ ID NO. 9; or (c) SEQ ID NO. 10; or (d) SEQ ID NO. 11.

In certain embodiments, the methods described herein are used to treat cancer. In certain embodiments, the method is used to treat an autoimmune disease. In certain embodiments, the method is used to treat an inflammatory condition. In certain embodiments, the method is used to prevent and treat graft rejection. In certain embodiments, the method is used to treat an infectious disease. In certain embodiments, the method further comprises administering at least one additional active agent; and, optionally, the additional active agent is an additional cytokine.

In certain embodiments, the methods described herein comprise: i) isolating an immune cell-containing sample; (ii) transducing or transfecting the immune cells with a nucleic acid sequence encoding the chimeric cytokine receptor; (iii) administering or infusing the immune cells from (ii) to the subject; and (iv) contacting the immune cells with the cytokine that binds the chimeric receptor. In certain embodiments, the subject has undergone an immuno-depletion treatment prior to administering or infusing the cells to the subject. In certain embodiments, the immune cell-containing sample is isolated from the subject that will be administered or infused with the cells. In certain embodiments, the immune cells are contacted with the cytokine in vitro prior to administering or infusing the cells to the subject. In certain embodiments, the immune cells are contacted with the cytokine that binds the chimeric receptor for a sufficient time to activate signaling from the chimeric receptor.

Described herein is a kit for treating a subject in need thereof, comprising: cells encoding a chimeric receptor described herein, and, optionally, the cells are immune cells; and instructions for use; and, optionally, the kit comprises a cytokine that binds the chimeric receptor. Described herein is a kit for producing a chimeric receptor expressed on a cell, comprising: an expression vector encoding a chimeric receptor described herein and instructions for use; and, optionally, the kit comprises a cytokine that binds the chimeric receptor.

Described herein is a kit for producing a chimeric receptor expressed on a cell, comprising: cells comprising an expression vector encoding a chimeric receptor described herein and, optionally, the cells are bacterial cells, and instructions for use; and, optionally, the kit comprises a cytokine that binds the chimeric receptor.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 is a diagram showing the structures of the Site II and III interfaces of the 2:2 G-CSF:G-CSFR heterodimeric complex.

FIG. 2 is a diagram outlining the strategy used for the G-CSF:G-CSFR interface design.

FIG. 3 is a graph showing the energetic components of site II interface interactions.

FIG. 4 is a diagram showing the structure of the site II interface and the interactions of Arg167 (left) and Arg141 (right) of G-CSFR(CRH) with G-CSF residues at site II interface.

FIG. 5 is a diagram showing wild type G-CSF at site II (left) and overlay of the same region of a triplicate ZymeCAD™ mean-field pack of site II design #35 (right).

FIG. 6 are images of SDS-PAGE showing results of G-CSF pulldown assays of G-CSF designs #: 6, 7, 8, 9, 15, 17, 30, 34, 35, and 36 (top panel) and co-expressed and purified site II design complexes #: 6, 7, 8, 9, 15, 17, 30, 34, 35, and 36 (bottom panel), first lane WT G-CSF control, second lane WT G-CSF:G-CSFR(CRH) control.

FIG. 7 are images of SDS-PAGE showing results of G-CSF pulldown assays of G-CSF designs #: 6, 7, 8, 9, 15, 17, 30, 34, 35, and 36 co-expressed with WT-G-CSFR (top panel) and G-CSF pulldown assays of WT G-CSF co-expressed with G-CSFR designs #: 6, 7, 8, 9, 15, 17, 30, 34, 35, and 36 (bottom panel, first lane WT:WT G-CSF:G-CSFR pulldown control.

FIG. 8 is a graph showing the energetic components of site III interface interactions.

FIG. 9 is a diagram showing the structure of the site III interface and interactions of R41 (left) and E93 (right) of G-CSFR(Ig) with G-CSF residues at site III interface.

FIG. 10 are images of SDS-PAGE showing results of G-CSF pulldown assays of designs #401 and 402 (top panel) and co-expressed and purified site II/III design complexes #401 and 402, and with WT G-CSFR and pulldown of WT G-CSF co-expressed with design #401 and 402 G-CSFR (bottom panel), first lane WT G-CSF control, second lane WT G-CSF:G-CSFR(Ig-CRH) control.

FIG. 11: are graphs showing size-exclusion chromatography profile of WT (SX75) G-CSF and design 130 (SX75), 303 (SX200) and 401 (SX200) G-CSF_{E} mutants post TEV cleavage.

FIG. 12: are graphs showing size-exclusion chromatography profile of purified WT (SX75) and purified design 401 (SX200) and 402 (SX200) G-CSFR(Ig-CRH)_{E} mutants post TEV cleavage.

FIG. 13: are graphs showing binding SPR sensorgrams for WT, designs #130, #401, #402 G-CSF, binding to their cognate or mispaired G-CSFR(Ig-CRH). Each G-CSF vs G-CSFR pair is labelled in the bottom of each panel. Representative steady state fit used to derive KDs for the cognate pair of design #401 and #402 are shown under their respective sensorgrams.

FIG. 14: are graphs showing: Top left panel: DSC thermograms of WT G-CSF, design #130 and #134 G-CSF_{E}; Top right panel: DSC thermograms of WT G-CSFR(Ig-CRH), design #130 and #134 G-CSFR(Ig-CRH)_{E}; Bottom left panel: DSC thermograms of design #401 and #402 G-CSF_{E}; Bottom right: DSC thermograms of design #300, #303, #304 and #307 G-CSF_{E}

FIG. 15: are graphs showing results of bromo-deoxyuridine (BrdU) assays showing proliferation 32D-IL-2RβIL2Rb cells expressing A) G-CSFR_{WT}-ICD_{IL-2Rb} (homodimer) or B) G-CSFR_{WT}-ICD_{IL-2Rb}+G-CSFR_{WT}-ICD_{gc} (heterodimer). Cells were stimulated with no cytokine, IL-2 (300 IU/ml) or G-CSF_{WT} (100 ng/ml in A or 30 ng/ml in B).

FIG. 16: are graphs showing results of BrdU assays showing proliferation of 32D-IL-2Rβ cells expressing A) G-CSFR₁₃₇-ICD_{gp130-IL-2Rb} (homodimer); or B) G-CSFR₁₃₇-ICD_{IL-2Rb}+G-CSFR₁₃₇-ICD_{gc} (heterodimer). Cells were stimulated with no cytokine, IL-2 (300 IUIUIU/ml), G-CSF_{WT} (30 ngngng/ml), or G-CSFR₁₃₇ (30 ng/ml).

FIG. 17: are graphs showing results of BrdU assays showing proliferation of 32D-IL-Rβ cells expressing: A) G-CSFR_{WT}-ICD_{gp130-IL-2Rb} (homodimer); or B) G-CSFR_{WT}-ICD_{IL-2Rb}+G-CSFR_{WT}-ICD_{gc} (heterodimer). Cells were stimulated with no cytokine, IL-2 (300 IU/ml), G-CSF_{WT} (30 ng/ml), or G-CSFR₁₃₇ (30 ng/ml).

FIG. 18: are western blots showing signaling of 32D-IL-2Rβ cells expressing: G-CSFR_{WT}-ICD_{gp130-IL-2Rb} (homodimer), G-CSFR₁₃₇-ICD_{gp130-IL-2Rb} (homodimer), G-CSFR_{WT}-ICD_{IL-2Rb}+G-CSFR_{WT}-ICD_{gc} (heterodimer) or G-CSFR₁₃₇-ICD_{IL-2Rb}+G-CSFR₁₃₇-ICD_{gc} (heterodimer). Cells were stimulated with no cytokine, IL-2 (300 IU/ml), G-CSF_{WT} (30 ng/ml), or G-CSFR₁₃₇ (30 ng/ml).

FIG. 19 presents graphs showing the results of BrdU incorporation assays to assess cell cycle progression of primary murine T cells expressing the indicated chimeric receptors (or non-transduced cells) in response to stimulation with no cytokine, IL-2 or WT, 130, 304 or 307 cytokine. A and B represent experimental replicates.

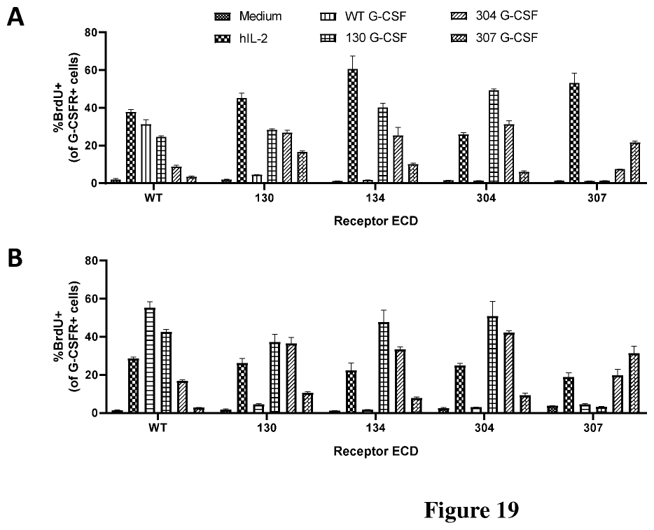

FIG. 20 is a schematic of native IL-2Rβ, IL-2Rγc, and G-CSFR subunits, as well as the G2R-1 receptor subunit designs.

FIG. 21 presents graphs showing the expansion (fold change in cell number) of 32D-IL-2Rβ cells (which is the 32D cell line stably expressing the human IL-2Rβ subunit) expressing the indicated G-CSFR chimeric receptor subunits and stimulated with WT G-CSF, IL-2 or no cytokine. G/γc was tagged at its N-terminus with a Myc epitope (Myc/G/γc), and G/IL-2Rβ was tagged at its N-terminus with a Flag epitope (Flag/G/IL-2Rβ); these epitope tags aid detection by flow cytometry and do not impact the function of the receptors. In addition, the lower panels in B-D show the percentage of cells expressing the G-CSFR ECD (% G-CSFR+) under each of the culture conditions. Squares represent cells stimulated with IL-2. Triangles represent cells stimulated with G-CSF. Circles represent cells not stimulated with a cytokine.

FIG. 22 presents graphs showing the expansion (fold change in cell number) of human T cells expressing the Flag-tagged G/IL-2Rβ subunit alone, the Myc-tagged G/γc subunit alone, or the full-length G-CSFR. A-D) PBMC-derived T cells; E-H) tumor-associated lymphocytes (TAL). Squares represent cells stimulated with IL-2. Triangles represent cells stimulated with G-CSF. Circles represent cells not stimulated with a cytokine.

FIG. 23 is a schematic of native and chimeric receptors, showing JAK, STAT, Shc, SHP-2 and PI3K binding sites. The shading scheme includes receptors from FIG. 1.

FIG. 24 is a schematic of chimeric receptors, showing Jak, STAT, Shc, SHP-2 and PI3K binding sites. The shading scheme includes receptors from FIGS. 1 and 4.

FIG. 25 is a diagram of the lentiviral plasmid containing the G2R-2 cDNA insert.

FIG. 26 presents graphs showing G-CSFR ECD expression assessed by flow cytometry in cells transduced with G2R-2. A) 32D-IL-2Rβ cell line; B) PBMC-derived human T cells and human tumor-associated lymphocytes (TAL).

FIG. 27 presents graphs showing the expansion (fold change in cell number) of cells expressing G2R-2 compared to non-transduced cells. A) Human PBMC-derived T cells; B, C) Human tumor-associated lymphocytes (TAL) from two independent experiments. Squares represent cells stimulated with IL-2. Triangles represent cells stimulated with G-CSF. Circles represent cells not stimulated with cytokine.

FIG. 28 presents graphs showing expansion (fold change in cell number) of CD4- or CD8-selected human tumor-associated lymphocytes expressing G2R-2 compared to non-transduced cells. A) Non-transduced CD4-selected cells; B) Non-transduced CD8-selected cells; C) CD4-selected cells transduced with G2R-2; D) CD8-selected cells transduced with G2R-2. Dotted gray line represents cells stimulated with IL-2. Solid black line represents cells stimulated with G-CSF. Dashed gray line represents cells not stimulated with a cytokine.

FIG. 29 presents graphs showing expansion (fold change in cell number) of CD4+ or CD8+ tumor-associated lymphocytes expressing G2R-2. The cells were initially expanded in G-CSF or IL-2, as indicated. Cells were then plated in either IL-2, G-CSF or medium only. Solid gray line represents cells stimulated with IL-2. Solid gray line represents cells stimulated with IL-2. Solid black line represents cells stimulated with G-CSF. Dashed light gray line represents cells expanded in IL-2 and then stimulated with medium only. Dashed dark gray line represents cells expanded in G-CSF and then stimulated with medium only.

FIG. 30 presents a graph showing immunophenotype (by flow cytometry) of CD4- or CD8-selected tumor-associated lymphocytes (TAL) expressing G2R-2 chimeric receptor construct versus non-transduced cells, after expansion in G-CSF or IL-2. A) Percentage of live cells showing a CD4+, CD8+ or CD3-CD56+ cell surface phenotype. B) Percentage of live cells showing the indicated cell surface phenotypes based on CD45RA and CCR7 expression.

FIG. 31 presents graphs showing the results of BrdU incorporation assays to assess proliferation of primary human T cells expressing G2R-2 versus non-transduced cells. T cells were selected by culture in IL-2 or G-CSF, as indicated, prior to the assay. A) Tumor-associated lymphocytes; B) PBMC-derived T cells.

FIG. 32 presents graphs showing the results of BrdU incorporation assays to assess proliferation of primary murine T cells expressing G2R-2 or the single-chain G/IL-2Rβ (a component of G2R-1) versus mock-transduced cells. A) Transduction efficiency as reflected by the percentage of cells expressing the G-CSFR ECD (by flow cytometry) after culture in the indicated cytokines; B) Percent BrdU incorporation in all live cells in response to the indicated cytokines; C) Percent BrdU incorporation by cells expressing the G-CSFR ECD (G-CSFR+ cells). All cells were expanded in IL-2 for 3 days prior to assay. Squares represent cells stimulated with IL-2. Triangles represent cells stimulated with G-CSF. Circles represent cells not stimulated with a cytokine.

FIG. 33 presents western blots to detect the indicated cytokine signaling events in human primary T cells expressing G2R-2 versus non-transduced cells. β-actin, total Akt and histone H3 serve as a protein loading controls. A, B) Tumor-associated lymphocytes (TALs); C) PBMC-derived T cells.

FIG. 34 presents western blots to detect the indicated cytokine signaling events in primary murine T cells expressing G2R-2 or the single-chain G/IL-2Rβ (from G2R-1) versus mock-transduced cells. Arrow indicates the specific phospho-Jak2 band, other larger bands are presumed to be the result of cross-reactivity of the primary anti-phospho-Jak2 antibody with phospho-Jak1. β-actin and histone H3 serve as protein loading controls.

FIG. 35 is a graph showing the results of a BrdU incorporation assay to assess cell cycle progression of 32D-IL-2Rβ cells expressing the indicated chimeric receptors (or non-transduced cells) in response to stimulation with no cytokine, IL-2 (300 IU/mL), WT G-CSF (30 ng/mL) or 130 G-CSF (30 ng/mL).

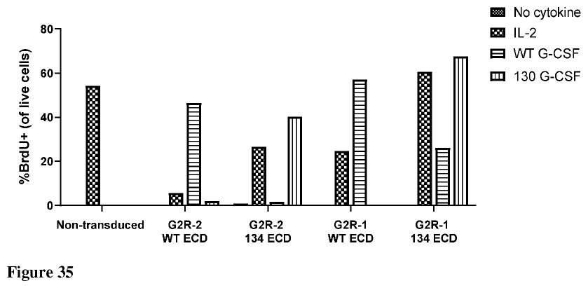

FIG. 36 presents graphs showing the results of BrdU incorporation assays to assess cell cycle progression of primary murine T cells expressing the indicated chimeric receptors (or non-transduced cells) in response to stimulation with no cytokine, IL-2 or WT, 130, 304 or 307 cytokine. A and B represent experimental replicates.

FIG. 37 presents western blots to detect the indicated cytokine signaling events in 32D-IL-2Rβ cells expressing the indicated chimeric receptor subunits (or non-transduced cells) in response to stimulation with no cytokine, IL-2, WT G-CSF or 130 G-CSF. β-actin and histone H3 serve as protein loading controls.

FIG. 38 presents A) western blots to detect the indicated cytokine signaling events in primary murine T cells expressing the indicated chimeric receptor subunits in response to stimulation with no cytokine, IL-2, WT G-CSF, 130 G-CSF or 304 G-CSF. β-actin and histone H3 serve as protein loading controls. B) Transduction efficiency of cells used in panel A, as assessed by flow cytometry with an antibody specific for the extracellular domain of the human G-CSF receptor.

FIG. 39 presents plots showing G-CSFR ECD expression by flow cytometry in primary human tumor-associated lymphocytes (TAL) transduced with the indicated chimeric receptor constructs. Live CD3+, CD56− cells were gated on CD8 or CD4, and G-CSFR ECD expression is shown for each population.

FIG. 40 presents graphs and images showing the expansion, proliferation and signaling of primary human tumor-associated lymphocytes (TAL) expressing G2R-3 versus non-transduced cells. A) Graph showing the results of a T-cell expansion assay, where cells were transduced with G2R-3-encoding lentivirus, washed, and re-plated in IL-2 (300 IU/ml), wildtype G-CSF (100 ng/ml) or no cytokine. Live cells were counted every 3-4 days. Squares represent cells stimulated with IL-2. Triangles represent cells stimulated with G-CSF. Circles represent cells not stimulated with a cytokine. B) Western blot to assess intracellular signaling events. Cells were harvested from the expansion assay and stimulated with IL-2 (300 IU/ml) or wildtype G-CSF (100 ng/ml). Arrow indicates the specific phospho-Jak2 band at 125 kDa; larger bands are presumed to be the result of cross-reactivity of the primary anti-phospho-Jak2 antibody with phospho-Jak1. β-actin and histone H3 serve as protein loading controls. C) Graph showing the results of a BrdU incorporation assay to assess T-cell proliferation. Cells were harvested from the expansion assay, washed, and re-plated in IL-2 (300 IU/ml), wildtype G-CSF (100 ng/ml) or no cytokine.

FIG. 41 presents graphs showing the fold expansion and G-CSFR ECD expression of primary human PBMC-derived T cells expressing G2R-3 with WT ECD versus non-transduced cells. A) Graph showing the results of a T-cell expansion assay, where cells were transduced with G2R-3-encoding lentivirus. On Day 1, WT G-CSF (100 ng/ml) or no cytokine (medium alone) were added to the culture. Thereafter, to Day 21, cells were replenished with medium containing WT G-CSF or no cytokine. On Day 21 of expansion, cells were washed and re-plated in WT G-CSF (100 ng/mL), IL-7 (20 ng/mL) and IL-15 (20 ng/mL), or no cytokine. Live cells were counted every 2-4 days. Squares represent cells stimulated with G-CSF. Triangles represent cells stimulated with G-CSF and re-plated in IL-7 and IL-15 on Day 21. Circles represent cells not stimulated with a cytokine. Diamonds represent cells stimulated with G-CSF, and re-plated in medium only on Day 21. B) Graph showing the expression of the G-CSFR ECD, as determined by flow cytometry, on Day 21 or 42 of expansion.

FIG. 42 presents graphs showing the intracellular signaling and immunophenotype of primary human PBMC-derived T cells expressing G2R-3 versus non-transduced cells. A) Western blot to assess intracellular signaling events. Cells were harvested from an expansion assay and stimulated with IL-2 (300 IU/ml) or wildtype G-CSF (100 ng/ml). β-actin serves as protein loading control. B, C) Representative flow cytometry plots and graphs showing the immunophenotype, assessed by flow cytometry, of cells expressing G2R-3 versus non-transduced cells on Day 42 of expansion.

FIG. 43 presents graphs showing the fold expansion of primary human PBMC-derived T cells expressing G2R-3 with 304 or 307 ECD versus non-transduced cells. A) Graph showing the results of a T-cell expansion assay, where cells were transduced with G2R-3 304 ECD-encoding lentivirus. B) Graph showing the results of a T-cell expansion assay, where cells were transduced with G2R-3 307 ECD-encoding lentivirus. C) Graph showing the results of a T-cell expansion assay with non-transduced cells. On Day 2 IL-2 (300 IU/mL), 304 G-CSF (100 ng/ml), 307 G-CSF (100 ng/mL) or no cytokine (medium alone) were added to the culture, as indicated, and replenished every two days thereafter. Live cells were counted every 3-4 days. Diamonds represent cells stimulated with 304 G-CSF. Squares represent cells stimulated with 307 G-CSF. Triangles represent cells stimulated with IL-2. Inverted triangles represent cells not stimulated with a cytokine.

FIG. 44 presents a graph showing the results of a BrdU incorporation assay to assess proliferation of primary human PBMC-derived T cells expressing G2R-3 with 304 or 307 ECD versus non-transduced cells. Cells were transduced with G2R-3 304 ECD- or 307 ECD-encoding lentivirus and expanded in the 304 or 307 G-CSF (100 ng/mL). Non-transduced cells were expanded in IL-2 (300 IU/mL). On Day 12 of expansion cells were washed, and re-plated in IL-2 (300 IU/ml), 130 G-CSF (100 ng/ml), 304 G-CSF (100 ng/ml), 307 G-CSF (100 ng/ml) or no cytokine.

FIG. 45 presents a graph showing G-CSFR ECD expression by flow cytometry in primary murine T cells transduced with the indicated chimeric receptor constructs.

FIG. 46 shows G-CSF-induced phosphorylation of STAT3 (detected by flow cytometry) in primary PBMC-derived human T cells expressing G21R-1 or G21R-2. Cells were subdivided (i.e., gated) into G-CSFR-positive (upper panels) or G-CSFR-negative (lower panels) populations.

FIG. 47 presents graphs and images showing G-CSF-induced biochemical signaling events in primary murine T cells expressing G21R-1 or G12R-1. A) Graph showing phosphorylation of STAT3 (detected by flow cytometry) in CD4+ or CD8+ cells transduced with G21R-1 and stimulated with no cytokine, IL-21 or G-CSF. B) Graph showing the percentage of cells staining positive for phospho-STAT3 after stimulation with no cytokine (black circles), IL-21 (squares) or WT G-CSF (gray circles). Live cells were gated on CD8 or CD4, and the percentage of phospho-STAT3-positive cells is shown for each population. C) Western blots to assess the indicated cytokine signaling events in cells expressing G21R-1 or G12R-1 and stimulated with IL-21, IL-12 or WT G-CSF. β-actin and histone H3 serve as protein loading controls.

FIG. 48 presents graphs and images showing proliferation, G-CSFR ECD expression and WT G-CSF-induced intracellular signaling events in primary murine T cells expressing G2R-2, G2R-3, G7R-1, G21/7R-1 and G27/2R-1, or mock-transduced T cells. A, B) Graphs showing the results of BrdU incorporation assays to assess T-cell proliferation. Cells were harvested, washed, and re-plated in IL-2 (300 IU/ml), wildtype G-CSF (100 ng/ml) or no cytokine. Panels A and B are experimental replicates. C) Graph showing G-CSFR ECD expression by flow cytometry in primary murine T cells transduced with the indicated chimeric receptor constructs. D) Western blots to assess the indicated cytokine signaling events in cells expressing G2R-2, G2R-3, G7R-1, G21/7R-1 and G27/2R-1, or mock-transduced T cells. Cells were stimulated with IL-2 (300 IU/mL), IL-7 (10 ng/mL), IL-21 (10 ng/mL), IL-27 (50 ng/mL) or G-CSF (100 ng/mL). β-actin and histone H3 serve as protein loading controls.

FIG. 49 presents graphs and images showing proliferation, G-CSFR ECD expression and G-CSF-induced biochemical signaling events in primary murine T cells expressing G21/2R-1, G12/2R-1 and 21/12/2R-1, or mock-transduced T cells. A, B) Graphs showing the results of BrdU incorporation assays to assess T-cell proliferation. Cells were harvested, washed, and re-plated in IL-2 (300 IU/ml), wildtype G-CSF (100 ng/ml) or no cytokine. Panels A and B are experimental replicates. C) Graph showing G-CSFR ECD expression by flow cytometry in primary murine T cells transduced with the indicated chimeric receptor constructs. D) Western blots to assess the indicated cytokine signaling events in cells expressing G21/2R-1, G12/2R-1 and 21/12/2R-1 or mock-transduced T cells. Cells were stimulated with IL-2 (300 IU/mL), IL-21 (10 ng/mL), IL-12 (10 ng/mL) or G-CSF (100 ng/mL). β-actin and histone H3 serve as protein loading controls.

FIG. 50 presents graphs showing the fold expansion and G-CSFR ECD expression of primary human PBMC-derived T cells expressing G12/2R-1 with 134 ECD, versus non-transduced cells. A) Graph showing the results of a T-cell expansion assay, where cells were transduced with G12/2R-1_134-ECD-encoding lentivirus and expanded in IL-2 (300 IU/mL), 130 G-CSF (100 ng/ml) or medium. Live cells were counted every 4-5 days. Squares represent cells not stimulated with a cytokine. Triangles represent cells stimulated with 130 G-CSF. Diamonds represent cells stimulated with IL-2. B) Graph showing the results of a T-cell expansion assay, where cells were transduced as in panel A. On Day 19 of expansion, cells were washed in re-plated in IL-2, 130 G-CSF or medium only. Live cells were counted every 4-5 days. Squares represent cells not stimulated with a cytokine. Light grey diamonds represent cells stimulated with 130 G-CSF. Dark grey diamonds represent cells stimulated with IL-2. Light grey inverted triangles represent cells initially stimulated with IL-2, and then re-plated in medium only on Day 19. Dark grey triangles represent cells initially stimulated with 130 G-CSF, and then re-plated in medium only on Day 19. C) Graph showing the expression of the G-CSFR ECD, as determined by flow cytometry, on Day 4 or 16 of expansion.

FIG. 51 presents graphs showing the proliferation and immunophenotype of primary human PBMC-derived T cells expressing G12/2R-1 with 134 ECD, versus non-transduced cells. A) Graph showing the results of a BrdU incorporation assay to assess T-cell proliferation. Cells were harvested, washed, and re-plated in IL-2 (300 IU/ml), IL-2+IL-12 (300 IU/ml and 10 ng/mL, respectively), 130 G-CSF (300 ng/ml) or medium alone. B, C) Representative flow cytometry plots and graph showing the immunophenotype, assessed by flow cytometry, of cells expressing G12/2R-1 with 134 ECD, versus non-transduced cells, on Day 16 of expansion.

FIG. 52 presents graphs showing the fold expansion and proliferation of primary human PBMC-derived T cells expressing G12/2R-1 with 304 ECD, versus non-transduced cells. A) Graph showing the results of a T-cell expansion assay, where cells were transduced with G12/2R-1_134-ECD-encoding lentivirus, and expanded in IL-2 (300 IU/mL), 130 G-CSF (100 ng/ml), 304 G-CSF (100 ng/ml) or medium alone. Non-transduced cells were cultured in IL-2, 130 G-CSF, 304 G-CSF or medium alone. Live cells were counted every 3-4 days. Inverted triangles represent cells not stimulated with cytokine. Triangles represent cells stimulated with IL-2. Circles represent cells stimulated with 130 G-CSF. Diamonds represent cells stimulated with 304 G-CSF. B) Cells were harvested from the expansion assay on Day 12, and were washed and re-plated in IL-2 (300 IU/ml), 130 G-CSF (300 ng/ml), 304 G-CSF (100 ng/ml), 307 G-CSF (100 ng/ml) or medium alone.

FIG. 53 presents western blots to detect the indicated cytokine signaling events in primary PBMC-derived T cells expressing G2R-3 with 304 ECD, G12/2R-1 with 304 ECD, or non-transduced T cells. Cells were harvested from the expansion assay and stimulated with 304 G-CSF (100 ng/mL), IL-2 (300 IU/mL), IL-2 and IL-12 (10 ng/mL), or medium alone, as indicated. Black arrows and small outcropped panel on the right indicate the molecular weight markers at 115 kDa and 140 kDa from a protein ladder. β-actin and histone H3 serve as protein loading controls.

### DETAILED DESCRIPTION OF THE INVENTION

Briefly, and as described in more detail below, described herein are methods and compositions for selective activation of cells using variant cytokine receptor and cytokine pairs, wherein the cytokine receptors comprise a variant extracellular domain (ECD) of granulocyte-colony stimulating factor receptor (G-CSFR). In certain embodiments, the methods and compositions described herein are useful for exclusive activation of cells for adoptive cell transfer therapy. Thus, included herein are methods for producing cells expressing variant receptors that are selectively activated by a cytokine that does not bind its native receptor. Also disclosed herein, are methods of treating a subject in need thereof, comprising administering to the subject a cell expressing receptors comprising a variant ECD of G-CSFR and co-administering a variant of G-CSF that binds to the variant ECD of G-CSFR. In certain aspects, the compositions and methods described herein address an unmet need for the selective activation of cells for adoptive cell transfer methods and may reduce or eliminate the need for immuno-depletion of a subject prior to adoptive cell transfer or for administering broad-acting stimulatory cytokines such as IL-2.

### Definitions

Terms used in the claims and specification are defined as set forth below unless otherwise specified.

The term “treatment” refers to any therapeutically beneficial result in the treatment of a disease state, e.g., a cancer disease state, including prophylaxis, lessening in the severity or progression, remission, or cure thereof.

The term “in vivo” refers to processes that occur in a living organism.

The term “mammal” as used herein includes both humans and non-humans and include but is not limited to humans, non-human primates, canines, felines, murines, bovines, equines, and porcines.

The term “sufficient amount” means an amount sufficient to produce a desired effect, e.g., an amount sufficient to selectively activate a receptor expressed on a cell.

The term “therapeutically effective amount” is an amount that is effective to ameliorate a symptom of a disease. A therapeutically effective amount can be a “prophylactically effective amount” as prophylaxis can be considered therapy.

The term “operatively linked” refers to nucleic acid or amino acid sequences that are placed into a functional relationship with another nucleic acid or amino acid sequence, respectively. Generally, “operatively linked” means that nucleic acid sequences or amino acid sequences being linked are contiguous, and, in the case of a secretory leader, contiguous and in reading phase.

As used herein, the term “extracellular domain” (ECD) refers to the domain of a receptor (e.g., G-CSFR) that, when expressed on the surface of a cell, is external to the plasma membrane. In certain embodiments the ECD of G-CSFR comprises at least a portion of SEQ ID NO. 2 or SEQ ID NO. 7.

As used herein, the term “intracellular domain” (ICD) refers to the domain of a receptor that is located within the cell when the receptor is expressed on a cell surface.

As used herein, the term “transmembrane domain” (TMD or TM) refers to the domain or region of a cell surface receptor that is located within the plasma membrane when the receptor is expressed on a cell surface.

The term “cytokine” refers to small proteins (about 5-20 kDa) that bind to cytokine receptors and can induce cell signaling upon binding to and activation of a cytokine receptor expressed on a cell. Examples of cytokines include, but are not limited to: interleukins, lymphokines, colony stimulating factors and chemokines.

The term “cytokine receptor” refers to receptors that bind to cytokines, including type 1 and type 2 cytokine receptors. Cytokine receptors include, but are not limited to, G-CSFR, IL-2R (Interleukin-2 receptor), IL-7R (Interleukin-7 receptor), IL-12R (Interleukin-12 Receptor), and IL-21R (Interleukin-21 Receptor).

The term “chimeric receptors,” as used herein, refers to a transmembrane receptor that is engineered to have at least a portion of at least one domain (e.g., ECD, ICD, TMD, or C-terminal region) that is derived from sequences of one or more different transmembrane proteins or receptors.

The term “Site II interface,” “Site II region,” “Site II interface region,” or “Site II,” as used herein, refers to the larger of the G-CSF:G-CSFR 2:2 heterodimer binding interfaces of G-CSF with G-CSFR, located at the interface between G-CSF and the Cytokine Receptor Homologous (CRH) domain of G-CSFR.

The term “Site III interface,” “Site III region,” “Site III interface region,” or “Site III,” as used herein, refers to the smaller of the G-CSF:G-CSFR 2:2 heterodimer binding interfaces of G-CSF with G-CSFR, and is located at the interface between G-CSF and the N-terminal Ig-like domain of G-CSFR.

The term “at least a portion of” or “a portion of,” as used herein, in certain aspects refers to greater than 75%, greater than 80%, greater than 90%, greater than 95%, greater than 99% of the length of contiguous nucleic acid bases or amino acids of a SEQ ID NO described herein. In certain aspects, at least a portion of a domain or binding site (e.g., ECD, ICD, transmembrane, C-terminal region or signaling molecule binding site) described herein can be greater than 75%, greater than 80%, greater than 90%, greater than 95%, greater than 99% identical to a SEQ ID NO. described herein.

The term “wild-type” refers to the native amino acid sequence of a polypeptide or native nucleic acid sequence of a gene coding for a polypeptide described herein. The wild-type sequence of a protein or gene is the most common sequence of the polypeptide or gene for a species for that protein or gene.

The terms “variant cytokine-receptor pair,” “variant cytokine and receptor pairs,” “variant cytokine and receptor design(s),” “variant cytokine-receptor switch,” or “orthogonal cytokine-receptor pair” refers to genetically engineered pairs of proteins that are modified by amino acid changes to (a) lack binding to the native cytokine or cognate receptor; and (b) to specifically bind to the counterpart engineered (variant) ligand or receptor.

The term “variant receptor,” or “orthogonal receptor” as used herein, refers to the genetically engineered receptor of a variant cytokine-receptor pair and includes chimeric receptors.

The term “variant ECD,” as used herein, refers to the genetically engineered extracellular domain of a receptor (e.g., G-CSFR) of a variant cytokine-receptor pair.

The term “variant cytokine,” “variant G-CSF,” or “orthogonal cytokine” as used herein, refers to the genetically engineered cytokine of a variant cytokine-receptor pair.

As used herein, “do not bind,” “does not bind” or “incapable of binding” refers to no detectable binding, or an insignificant binding, i.e., having a binding affinity much lower than that of the natural ligand.

The term “selectively activates,” or “selective activation,” as used herein when referring to a cytokine and a variant receptor, refers to a cytokine that binds preferentially to a variant receptor and the receptor is activated upon binding of a cytokine to the variant receptor. In certain aspects, the cytokine selectively activates a chimeric receptor that has been co-evolved to specifically bind the cytokine. In certain aspects, the cytokine is a wild-type cytokine and it selectively activates a chimeric receptor that is expressed on cells, whereas the native, wild-type receptor to the cytokine is not expressed in the cells.

The term, “enhanced activity,” as used herein, refers to increased activity of a variant receptor expressed on a cell upon stimulation with a variant cytokine, wherein the activity is an activity observed for a native receptor upon stimulation with a native cytokine.

The term “immune cell” refers to any cell that is known to function to support the immune system of an organism (including innate and adaptive immune responses), and includes, but is not limited to, Lymphocytes (e.g., B cells, plasma cells and T cells), Natural Killer Cells (NK cells), Macrophages, Monocytes, Dendritic cells, Neutrophils, and Granulocytes. Immune cells include stem cells, immature immune cells and differentiated cells. Immune cells also include any sub-population of cells, however rare or abundant in an organism. In certain embodiments, an immune cell is identified as such by harboring known markers (e.g., cell surface markers) of immune cell types and sub-populations.

The term “T cells” refers to mammalian immune effector cells that may be characterized by expression of CD3 and/or T cell antigen receptor, which cells may be engineered to express an orthologous cytokine receptor. In some embodiments, the T cells are selected from naïve CD8⁺ T cells, cytotoxic CD8⁺ T cells, naïve CD4⁺ T cells, helper T cells, e. g., T_{H}2, T_{H}9, T_{H}11, T_{H}22, T_{FH}; regulatory T cells, e.g., T_{R}1, natural T_{Reg}, inducible T_{Reg}; memory T cells, e.g., central memory T cells, effector memory T cells, NKT cells, and γδT cells.

The term “G-CSFR” refers to Granulocyte Colony-Stimulating Factor Receptor. G-CSFR can also be referred to as: GCSFR, G-CSF Receptor, Colony Stimulating Factor 3 Receptor, CSF3R, CD114 Antigen, or SCN7. Human G-CSFR is encoded by the gene having an Ensembl identification number of: ENSG00000119535. Human G-CSFR is encoded by the cDNA sequence corresponding to GeneBank Accession number NM_156039.3.

The term “G-CSF” refers to Granulocyte Colony Stimulating Factor. G-CSF can also be called Colony Stimulating Factor 3 and CSF3. Human G-CSF is encoded by the gene having an Ensembl identification number of: ENSG00000108342. Human G-CSF is encoded by the cDNA sequence corresponding to GeneBank Accession number KP271008.1.

“JAK” can also be referred to as Janus Kinase. JAK is a family of intracellular, nonreceptor tyrosine kinases that transduce cytokine-mediated signals via the Jak-STAT pathway and includes JAK1, JAK2, JAK3 and TYK2. Human JAK1 is encoded by the gene having an Ensembl identification number of: ENSG00000162434. Human JAK1 is encoded by the cDNA sequence corresponding to GeneBank Accession number NM_002227. Human JAK2 is encoded by the gene having an Ensembl identification number of: ENSG00000096968. Human JAK2 is encoded by the cDNA sequence corresponding to GeneBank Accession number NM_001322194. Human JAK3 is encoded by the gene having an Ensembl identification number of: ENSG00000105639. Human JAK3 is encoded by the cDNA sequence corresponding to GeneBank Accession number NM_000215. Human_TYK2 is encoded by the gene having an Ensembl identification number of: ENSG00000105397. Human TYK2 is encoded by the cDNA sequence corresponding to GeneBank Accession number NM_001385197.

STAT can also be referred to as Signal Transducer and Activator of Transcription. STAT is a family of 7 STAT proteins: STAT1, STAT2, STAT3, STAT4, STAT5A, STAT5B and STAT6. Human STAT1 is encoded by the gene having an Ensembl identification number of: ENSG00000115415. Human STAT1 is encoded by the cDNA sequence corresponding to GeneBank Accession number NM_007315. Human_STAT2 is encoded by the gene having an Ensembl identification number of: ENSG00000170581. Human STAT2 is encoded by the cDNA sequence corresponding to GeneBank Accession number NM_005419. Human_STAT3 is encoded by the gene having an Ensembl identification number of: ENSG00000168610. Human STAT3 is encoded by the cDNA sequence corresponding to GeneBank Accession number NM_139276. Human STAT4 is encoded by the gene having an Ensembl identification number of: ENSG00000138378. Human STAT4 is encoded by the cDNA sequence corresponding to GeneBank Accession number NM_003151. Human STAT5A is encoded by the gene having an Ensembl identification number of: ENSG00000126561. Human STAT5A is encoded by the cDNA sequence corresponding to GeneBank Accession number NM_003152. Human STAT5B is encoded by the gene having an Ensembl identification number of: ENSG00000173757. Human STAT5B is encoded by the cDNA sequence corresponding to GeneBank Accession number NM_012448. Human STAT6 is encoded by the gene having an Ensembl identification number of: ENSG00000166888. Human STAT6 is encoded by the cDNA sequence corresponding to GeneBank Accession number NM_003153.

SHC can also be referred to as Src Homology 2 Domain Containing Transforming Protein. Shc is a family of three isoforms and includes p66Shc, p52Shc and p46Shc, SHC1, SHC2 and SHC3. Human SHC1 is encoded by the gene having an Ensembl identification number of: ENSG00000160691. Human SHC1 is encoded by the cDNA sequence corresponding to GeneBank Accession number NM_183001. Human SHC2 is encoded by the gene having an Ensembl identification number of: ENSG00000129946. Human SHC2 is encoded by the cDNA sequence corresponding to GeneBank Accession number NM_012435. Human SHC3 is encoded by the gene having an Ensembl identification number of: ENSG00000148082. Human SHC3 is encoded by the cDNA sequence corresponding to GeneBank Accession number NM_016848.

SHP-2 can also be referred to as Protein Tyrosine Phosphatase Non-Receptor Type 11 (PTPN11) and Protein-Tyrosine Phosphatase 1D (PTP-1D). Human SHP-2 is encoded by the gene having an Ensembl identification number of: ENSG00000179295. Human SHP-2 is encoded by the cDNA sequence corresponding to GeneBank Accession number NM_001330437.

PI3K can also be referred to as Phosphatidylinositol-4,5-Bisphosphate 3-Kinase. The catalytic subunit of PI3K can be referred to as PIK3CA. Human PIK3CA is encoded by the gene having an Ensembl identification number of: ENSG00000121879. Human PIK3CA is encoded by the cDNA sequence corresponding to GeneBank Accession number NM_006218.

Abbreviations used in this application include the following: ECD (extracellular domain), ICD (intracellular domain), a G-CSFR (Granulocyte-Colony Stimulating Factor Receptor), a G-CSF (Granulocyte-Colony Stimulating Factor), IL-2R (Interleukin-2 receptor), IL-12R (Interleukin 12 Receptor), IL-21R (Interleukin-21 Receptor) and IL-7R (Interleukin-7 receptor). IL-2Rγ can also be referred to herein as: IL-2RG, IL-2Rgc, γc, or IL-2Rγc. For select chimeric cytokine receptor designs: “G-CSFRwt-ICDIL-2Rb” is herein also referred to as “G/IL-2Rb”; “G-CSFRwt-ICDgc” is herein also referred to as “G/gc”; “G-CSFR137-ICDgp130-IL-2Rb” is herein also referred to as “G2R-2 with 137 ECD”; and “G-CSFR137-ICDIL-2Rb+GCSFR137-ICDgc” is herein also referred to as “G2R-1 with 137 ECD”.IL-2Rγ (i.e., IL-2RG, IL-2Rgc, γc, or IL-2Rγc).

It must be noted that, as used in the specification and the appended claims, the singular forms “a,” “an” and “the” include plural referents unless the context clearly dictates otherwise.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Variant Cytokine and Receptor Designs

Described herein are variant cytokine and receptor pairs for selective activation of the variant receptors. The variant receptors of the instant disclosure comprise an extracellular domain of G-CSFR; and the variant cytokine comprises a G-CSF (Granulocyte Colony-Stimulating Factor), which binds to and activates the variant receptor. In certain embodiments, the variant receptors are chimeric receptors comprising an ECD of G-CSFR and at least a portion of an ICD of a receptor different from G-CSFR.

Variant G-CSF and Variant G-CSFR ECD Pairs

In certain aspects, the variant G-CSF and receptor designs described herein comprise at least one Site II interface region mutation, at least one Site III interface region mutation, and combinations thereof. In certain aspects, the variant G-CSF and receptor designs described herein comprise at least one Site II or Site III interface region mutation listed in Tables 2, 4 or 6.

In certain aspects, at least one mutation on the variant receptor in the site II interface region is located at an amino acid position of the G-CSFR extracellular domain selected from the group consisting of amino acid position: 141, 167, 168, 171, 172, 173, 174, 197, 199, 200, 202 and 288 of the G-CSFR extracellular domain (SEQ ID NO. 2).

In certain aspects, at least one mutation on the variant G-CSF site II interface region is located at an amino acid position of the G-CSF selected from the group consisting of amino acid position: 12, 16, 19, 20, 104, 108, 109, 112, 115, 116, 118, 119, 122 and 123 of G-CSF (SEQ ID NO. 1).

In certain aspects, at least one mutation on the variant receptor site II interface region is selected from the group of mutations of the G-CSFR extracellular domains consisting of: R141E, R167D, K168D, K168E, L171E, L172E, Y173K, Q174E, D197K, D197R, M199D, D200K, D200R, V202D, R288D, and R288E.

In certain aspects, at least one mutation on the variant G-CSF site II interface region is selected from the group of mutations of G-CSF consisting of: K16D, R, S12E, S12K, S12R, K16D, L18F, E19K, E19R, Q20E, D104K, D104R, L108K, L108R, D109R, D112R, D112K, T115E, T115K, T116D, Q119E, Q119R, E122K, E122R, and E123R.

In certain aspects, at least one mutation on the variant site III interface region is selected from the group of mutations of the G-CSFR extracellular domain selected for the group consisting of amino acid position: 30, 41, 73, 75, 79, 86, 87, 88, 89, 91, and 93 of SEQ ID NO. 2.

In certain aspects, at least one mutation on the variant site III interface region is selected from the group of mutations of the G-CSF selected for the group consisting of amino acid position: 38, 39, 40, 41, 46, 47, 48, 49, and 147 of SEQ ID NO. 1.

In certain aspects, at least one mutation on the variant receptor site III interface region is selected from the group of mutations of the G-CSFR extracellular domains consisting of S30D, R41E, Q73W, F75K, S79D, L86D, Q87D, I88E, L89A, Q91D, Q91K, and E93K.

In certain aspects, at least one mutation on the variant G-CSF site III interface region is selected from the group of mutations of G-CSF consisting of: T38R, Y39E, K40D, K40F, L41D, L41E, L41K, E46R, L47D, V48K, V48R, L49K, and R147E.

The variant cytokine and receptor pairs described herein can comprise mutations in either the site II region alone, the site III region alone or both the site II and site III regions.

The variant cytokine and receptor pairs described herein can have any number of site II and/or site III mutations described herein. In certain aspects, the variant G-CSF and receptors have the mutations listed in Table 6. In certain aspects, the variant receptor and/or variant G-CSF may have 1, 2, 3, 4, 5, 6, 7, 8, 9, 10 or more mutations described herein.

In certain aspects, the variant receptors described herein comprise G-CSFR ECD domains that share at least 50%, at least 60%, at least 70%, at least 80%, at least 85%, at least 90%, at least 95%, at least 96%, at least 97%, at least 98%, or at least 99% amino acid identity to a G-CSFR ECD SEQ ID NO. described herein. In certain aspects, the chimeric receptor comprises the ECD of G-CSFR having an amino acid sequence of SEQ ID NO. 2, 3, 6 or 8.

In certain aspects, the variant G-CSF described herein comprise an amino acid sequence that shares at least 50%, at least 60%, at least 70%, at least 80%, at least 85%, at least 90%, at least 95%, at least 96%, at least 97%, at least 98%, or at least 99% amino acid identity to a G-CSFR ECD SEQ ID NO. 1.

In certain aspects, the ECD of G-CSFR comprises at least one amino acid substitution selected from the group consisting of R41E, R141E, and R167D.

A variant cytokine and/or receptor may be produced recombinantly not only directly, but also as a fusion polypeptide with a heterologous polypeptide, e.g. a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders. In certain embodiments, the signal sequence is the signal sequence of G-CSFR or GM-CSFR. In certain embodiments, the signal sequence is SEQ ID NO: 11 or SEQ ID NO: 12.

In certain as aspects, the variant receptor and/or variant G-CSF are modified (e.g., sugar groups, PEG) either naturally or synthetically to enhance stability. For example, in certain embodiments, variant cytokines are fused to the Fc domain of IgG, albumin, or other molecules to extend its half-life, e.g., by pegylation, glycosylation, and the like as known in the art. Fc-fusion can also promote alternative Fc receptor mediated properties in vivo. The “Fc region” can be a naturally occurring or synthetic polypeptide that is homologous to an IgG C-terminal domain produced by digestion of IgG with papain. IgG Fc has a molecular weight of approximately 50 kDa. The variant cytokines can include the entire Fc region, or a smaller portion that retains the ability to extend the circulating half-life of a chimeric polypeptide of which it is a part. In addition, full-length or fragmented Fc regions can be variants of the wild-type molecule.

Upon binding of the variant cytokine to the variant receptor, the variant receptor activates signaling that is transduced through native cellular elements to provide for a biological activity that mimics that native response, but which is specific to a cell engineered to express the variant receptor. In certain aspects, the variant receptor and G-CSF pair do not bind their native, wild-type G-CSF or native, wild-type G-CSFR. Thus, in certain embodiments, the variant receptor does not bind to the endogenous counterpart cytokine, including the native counterpart of the variant cytokine, while the variant cytokine does not bind to any endogenous receptors, including the native counterpart of the variant receptor In certain embodiments, the variant cytokine binds the native receptor with significantly reduced affinity compared to binding of the native cytokine to the native cytokine receptor. In certain embodiments, the affinity of the variant cytokine for the native receptor is less than 10×, less than 100×, less than 1,000× or less than 10,000× of the affinity of the native cytokine to the native cytokine receptor. In certain embodiments, the variant cytokine binds the native receptor with a K_{D} of greater than 1×10⁻⁴ M, 1×10⁻⁵ M, greater than 1×10⁻⁶ M; greater than 1×10⁻⁷ M, greater than 1×10⁻⁸ M, or greater than 1×10⁻⁹ M. In certain embodiments, the variant cytokine receptor binds the native cytokine with significantly reduced affinity compared to the binding of the native cytokine receptor to the native cytokine. In certain embodiments, the variant cytokine receptor binds the native cytokine less than 10×, less than 100×, less than 1,000× or less than 10,000× the native cytokine to the native cytokine receptor. In certain embodiments, the variant cytokine receptor binds the native cytokine with a K_{D} of greater than 1×10⁻⁴ M, 1×10⁻⁵ M, greater than 1×10⁻⁶ M; or greater than 1×10⁻⁷ M, greater than 1×10⁻⁸ M, or greater than 1×10⁻⁹ M. In some embodiments, the affinity of the variant cytokine for the variant receptor is comparable to the affinity of the native cytokine for the native receptor, e.g. having an affinity that is least about 1% of the native cytokine receptor pair affinity, at least about 5%, at least about 10%, at least about 25%, at least about 50%, at least about 75%, at least about 100%, and may be higher, e.g. 2×, 3×, 4×, 5×, 10× or more of the affinity of the native cytokine for the native receptor. The affinity can be determined by any number of assays well known to one of skill in the art. For example, affinity can be determined with competitive binding experiments that measure the binding of a receptor using a single concentration of labeled ligand in the presence of various concentrations of unlabeled ligand. Typically, the concentration of unlabeled ligand varies over at least six orders of magnitude. Through competitive binding experiments, IC₅₀ can be determined. As used herein, “IC₅₀” refers to the concentration of the unlabeled ligand that is required for 50% inhibition of the association between receptor and the labeled ligand. IC₅₀ is an indicator of the ligand-receptor binding affinity. Low IC₅₀ represents high affinity, while high IC₅₀ represents low affinity.

Binding of a variant cytokine to the variant cytokine receptor expressed on the surface of a cell, may or may not affect the function of the variant cytokine receptor (as compared to native cytokine receptor activity); native activity is not necessary or desired in all cases. In certain embodiments, the binding of an variant cytokine to the variant cytokine receptor will induce one or more aspects of native cytokine signaling. In certain embodiments, the binding of a variant cytokine to the variant cytokine receptor expressed on the surface of a cell causes a cellular response selected from the group consisting of proliferation, viability and enhanced activity.

TABLE 1
Sequence of human WT G-CSF and human WT G-CSFR Ig-CRH domain

| | | | | SEQ |
|---|---|---|---|---|
| | Uniprot | Residue | | ID |
| Protein | ID | numbering | Polypeptide Sequence | NO. |
| G-CSF | A0A0B4U5E3 | 1-174 | TPLGPASSLPQSFLLKCLEQVRKIQGDGAALQ | 1 |
| | | | EKLCATYKLCHPEELVLLGHSLGIPWAPLSSC | |
| | | | PSQALQLAGCLSQLHSGLFLYQGLLQALEGIS | |
| | | | PELGPTLDTLQLDVADFATTIWQQMEELGM | |
| | | | APALQPTQGAMPAFASAFQRRAGGVLVASH | |
| | | | LQSFLEVSYRVLRHLAQP | |
| Human | | | MTPLGPASSLPQSFLLKCLEQVRKIQGDGAA | 90 |
| G-CSF | | | LQEKLCATYKLCHPEELVLLGHSLGIPWAPL | |
| recomb- | | | SSCPSQALQLAGCLSQLHSGLFLYQGLLQAL | |
| inantly | | | EGISPELGPTLDTLQLDVADFATTIWQQMEEL | |
| produced | | | GMAPALQPTQGAMPAFASAFQRRAGGVLVA | |
| in E. | | | SHLQSFLEVSYRVLRHLAQP | |
| coli | | | | |
| G- | Q99062 | Ig: 2-97 | ECGHISVSAPIVHLGDPITASCIIKQNCSHLDP | 2 |
| CSFR | | CRH: 98-308 | EPQILWRLGAELQPGGRQQRLSDGTQESIITL | |
| (Ig-CRH) | | | PHLNHTQAFLSCSLNWGNSLQILDQVELRAG | |
| | | | YPPAIPHNLSCLMNLTTSSLICQWEPGPETHL | |
| | | | PTSFTLKSFKSRGNCQTQGDSILDCVPKDGQS | |
| | | | HCSIPRKHLLLYQNMGIWVQAENALGTSMSP | |
| | | | QLCLDPMDVVKLEPPMLRTMDPSPEAAPPQ | |
| | | | AGCLQLSWEPWQPGLHINQKCELRHKPQRG | |
| | | | EASWALVGPLPLEALQYELCGLLPATAYTLQ | |
| | | | IRCIRWPLPGHWSDWSPSLELRTTE | |

TABLE 2
Site II designs with mutations for G-CSF_{E} and G-CSFR_{E}.

| Design | Mutations G-CSF_{E} | Mutations G-CSFR_{E} |
|---|---|---|
| 1 | E19K | R288E |
| 2 | S12K_E19K | R288E |
| 6 | D104K | K168D |
| 7 | D104R_L108R_T115K | 168D_L171E_Q174E |
| 8 | S12R_L18F_D104R_L108K_D112R | R167D_K168D_D197E |
| 9 | D104R_L108K_D112R_Q119R_E122K | R141E_R167D_K168D_Q174D |
| 15 | K16D_T115E_T116D_Q119E | L171R_Y173V_D197R |
| 17 | E122R_E123R | R141E |
| 18 | E19K_Q20E | Y173K_R288E |
| 19 | K16E_E19K | D200R_R288E |
| 20 | E19K_D104K | K168D_R288E |
| 21 | E19K_D112R | L172E_R288E |
| 22 | E19K_D104K | K168E_M199D_R288D |
| 23 | S12E_K16D_E19K | D197K_D200K_R288E |
| 24 | E19R_D112K | R167D_V202D_R288E |
| 25 | E19K_D109R_D112R | R167D_M199D_R288D |
| 27 | E19R_D104R_L108K_D112R | R167D_K168D_V202E_I239V_R288E |
| 28 | E19K_D109K_D112R_E122R_E123R | R141E_R167D_M199D_R288E |
| 30 | L108K D112R | R167D |
| 34 | L108K_D112R_E122R_E123R | R141E_R167D |
| 35 | T115K_E122R_E123R | R141E_L171E_Q174E |
| 36 | S12E_K16E_T115E_Q119E | L171R_D197K_D200R |
| 42 | E19K_D109K_D112R | R167D_L172E_M199D_R288D |
| 43 | E19R_D104R_D112K | R167D_K168D_V202D_R288E |
| 44 | E19K_D104R_L108R_D112R | R167D_K168D_R288D |
| 45 | E19K_D112K_T115K | R167E_L171E_Q174E_R288E |
| 46 | E19K_D104K_E122R_E123R | R141E_K168D_R288E |
| 47 | E19R_E122R_E123R | R141E_V202E_I239V_R288E |
| 48 | E19K_D109K_D112R | R167F_L172E_M199D_R288D |
| 49 | K16E_E19R_T115E_Q119E | L171R_D197K_D200K_R288E |
| 50 | E19K_D104R_L108K_D112R | R167D_K168D_V202E_R288E |
| 80 | D112K T115K | R167E Q174E |
| 81 | E19K | R288D |
| 82 | L108K_D112R | R141E_R167D |
| 83 | E19R_D112K | R167D_R288E |

TABLE 4
Site III designs.

| Design # | Mutations G-CSF_{E} | Mutations G-CSFR_{E} |
|---|---|---|
| 51 | E46R_L49K | S30D_R41E |
| 52 | K40D_L41D | F75K_Q91K |
| 53 | L41K_E46R | R41E_Q91D |
| 54 | L41E_L47D | I88K |
| 55 | T38R_E46R | R41E_Q73E |
| 56 | K40D_R147E | F75K_E93K |
| 57 | E46R_V48R | R41E_Q87E |
| 58 | E46R | R41E_L86D |
| 59 | K40D_E46R | R41E_F75K |
| 60 | E46R | R41E_I88D |
| 61 | E46R_L49F | R41E_L89A |
| 62 | L41D | Q91K |
| 63 | T38R | Q73E |
| 64 | Y39E_K40D | F75K |
| 65 | L41K | I88E_Q91D |
| 66 | E46R | R41E |
| 67 | E46R | R41E_S79D |
| 68 | K40D | F75K |
| 69 | V48K | Q87D |
| 70 | R147E | E93K |
| 71 | L41K | Q91D |
| 72 | K40F | F75K |

TABLE 6
Examples of designs resulting from combinations of site II and III designs.

| | | | Parental | Parental |
|---|---|---|---|---|
| Design | | | site II | site III |
| Number | Mutations G-CSF_{E} | Mutations G-CSFR_{E} | design | design |
| 106 | E46R_D104K | R41E_K168D | 6 | 66 |
| 117 | E46R_E122R_E123R | R41E_R141E | 17 | 66 |
| 130 | E46R_L108K_D112R | R41E_R167D | 30 | 66 |
| 134 | E46R_L108K_D112R_E122R_E123R | R41E_R141E_R167D | 34 | 66 |
| 135 | E46R_T115K_E122R_E123R | R41E_R141E_L171E_Q174E | 35 | 66 |
| 137 | E46R_L108K_D112R | R41E_R141E_R167D | 82 | 66 |
| 300 | K40D_L41D_L108K_D112R | F75K_Q91K_R167D | 30 | 52 |
| 301 | T38R_E46R_L108K_D112R | R41E_Q73E_R167D | 30 | 55 |
| 302 | E46R_L108K_D112R | R41E_L86D_R167D | 30 | 58 |
| 303 | L108K D112R_R147E | E93K_R167D | 30 | 70 |
| 304 | E46R_L108K_D112R_R147E | R41E_E93K_R167D | 30 | 66, 70 |
| 305 | E19K_E46R_L108K_D112R | R41E_R167D_R288E | 1, 30 | 66 |
| 307 | S12E_K16D_E19K_E46R | R41E_D197K_D200K_R288E | 23 | 66 |
| 308 | E19R_E46R_D112K | R41E_R167D_V202D_R288E | 24 | 66 |
| 400 | E19K_E46R_D109R_D112R | R41E_R167D_M199D_R288D | 25 | 66 |
| 401 | E19K_E46R_L108K_D112R | R41E_R167D_R288D | 30, 81 | 66 |
| 402 | E19K_E46R_D112K_T115K | R41E_R167E_Q174E_R288E | 1, 80 | 66 |
| 403 | E19R_E46R_D112K | R41E_R167D_R288E | 83 | 66 |

Chimeric Receptors

In certain aspects, the variant receptors described herein are chimeric receptors. A chimeric receptor can comprise any of the variant G-CSFR ECD domains described herein. In certain aspects, the chimeric receptor further comprises at least a portion of the intracellular domain (ICD) of a different cytokine receptor. The intracellular domain of the different cytokine receptor can be selected from the group consisting of: gp130 (glycoprotein 130), IL-2Rβ or IL-2Rb (interleukin-2 receptor beta), IL-2Rγ or γc or IL-2RG (interleukin-2 receptor gamma), IL-7Rα (interleukin-7 receptor alpha), IL-12Rβ₂ (interleukin-12 receptor beta 2) and IL-21R (interleukin-21 receptor). In certain aspects, at least a portion of the intracellular domain comprises an amino acid sequence that shares at least 70%, at least 80%, at least 85%, at least 90%, at least 95%, at least 96%, at least 97%, at least 98%, or at least 99% amino acid identity to the amino acid sequence of a cytokine receptor ICD described herein. In certain aspects, at least a portion of a cytokine receptor ICD shares at least 70%, at least 80%, at least 85%, at least 90%, at least 95%, at least 96%, at least 97%, at least 98%, or at least 99% amino acid sequence identity to SEQ ID NO. 4, 7 or 9.

In certain embodiments, described herein are chimeric cytokine receptors comprising an extracellular domain (ECD) of a G-CSFR (Granulocyte-Colony Stimulating Factor Receptor) operatively linked to a second domain; the second domain comprising at least a portion of an intracellular domain (ICD) of a multi-subunit cytokine receptor, e.g., IL-2R. In certain aspects, the chimeric cytokine receptor comprises a portion of an ICD from Table 15A and Table 15B. In certain aspects, the chimeric cytokine receptor comprises a transmembrane domain selected from Table 15A and Table 15B. In certain aspects, the chimeric cytokine receptor ICD comprises Box1 and Box 2 regions from Table 15A, Table 15B and Table 16. In certain aspects, the chimeric cytokine receptor comprises at least one signaling molecule binding site from Table 15A, Table 15B and Table 16.

In certain aspects, the chimeric receptors described herein comprise amino acid sequences in N-terminal to C-terminal order of the sequences disclosed in each of Tables 17-20. In certain aspects, the sequences of the chimeric receptors described herein comprise nucleic acid sequences in 5′ to 3′ order of the sequences disclosed in each of Tables 17-20. In certain aspects, the chimeric cytokine receptor shares at least 50%, at least 60%, at least 70%, at least 80%, at least 85%, at least 90%, at least 95%, at least 96%, at least 97%, at least 98%, or at least 99% amino acid identity to the amino acid sequences in N-terminal to C-terminal order of the amino acid sequences disclosed in each of Tables 17-20. In certain aspects, the chimeric cytokine receptor shares at least 50%, at least 60%, at least 70%, at least 80%, at least 85%, at least 90%, at least 95%, at least 96%, at least 97%, at least 98%, or at least 99% nucleic acid identity to the nucleic acid sequences in 5′ to 3′ order of the nucleic acid sequences disclosed in each of Tables 17-20.

In certain aspects, the chimeric receptors described herein comprise at least a portion of an ICD of a cytokine receptor that shares at least 80%, at least 85%, at least 90%, at least 95%, at least 96%, at least 97%, at least 98%, or at least 99% amino acid or nucleic acid sequence identity to an ICD SEQ ID NO. described herein. In certain aspects, the chimeric receptor comprises at least a portion of an ICD of IL-2Rβ having an amino acid sequence of SEQ ID NO. 26, 29, 31, 39, 41, 43, 45, 47, or 49. In certain aspects, the chimeric receptor comprises at least a portion of an ICD of IL-2Rβ, i.e., IL-2Rb, having a nucleic acid sequence of SEQ ID NO. 54, 57, 59, 67, 69, 71, 73, 75, or 77. In certain aspects, the chimeric receptor comprises at least a portion of an ICD of IL-7Rα having an amino acid sequence of SEQ ID NO. 51 or a nucleic acid sequence of SEQ ID NO. 79. In certain aspects, the chimeric receptor comprises at least a portion of an ICD of IL-7R having an amino acid sequence of SEQ ID NO. 53 or a nucleic acid sequence of SEQ ID NO. 81. In certain aspects, the chimeric receptor comprises at least a portion of an ICD of IL-21R having an amino acid sequence of SEQ ID NO. 45 or 55. In certain aspects, the chimeric receptor comprises at least a portion of an ICD of IL-21R having a nucleic acid sequence of SEQ ID NO. 35 or 37. In certain aspects, the chimeric receptor comprises at least a portion of an ICD of IL-12Rβ₂ having an amino acid sequence of SEQ ID NO. 33, 42, or 46. In certain aspects, the chimeric receptor comprises at least a portion of an ICD of IL-12Rβ₂ having a nucleic acid sequence of SEQ ID NO. 61, 70 or 74. In certain aspects, the chimeric receptor comprises at least a portion of an ICD of G-CSFR having an amino acid sequence of SEQ ID NO. 30, 32, 34, 36, 38, 40, 44, 50 or 52. In certain aspects, the chimeric receptor comprises at least a portion of an ICD of G-CSFR having a nucleic acid sequence of SEQ ID NO. 58, 60, 62, 64, 66, 68, 72, 78 or 80. In certain aspects, the chimeric receptor comprises at least a portion of an ICD of gp130 having an amino acid sequence of SEQ ID NO. 28 or 48. In certain aspects, the chimeric receptor comprises at least a portion of an ICD of gp130 having a nucleic acid sequence of SEQ ID NO. 56 or 76. In certain aspects, the chimeric receptor comprises at least a portion of an ICD of IL-2Rγ (i.e., IL-2RG, IL-2Rgc, γc, or IL-2Rγc) having an amino acid sequence of SEQ ID NO. 27. In certain aspects, the chimeric receptor comprises at least a portion of an ICD of IL-2Rγ (i.e., IL-2RG, IL-2Rgc, γc, or IL-2Rγc) having a nucleic acid sequence of SEQ ID NO. 55.

In certain aspects, at least a portion of the ICDs described herein comprise at least one signaling molecule binding site. In certain aspects, at least one signaling molecule binding site is a STAT3 binding site of G-CSFR; a STAT3 binding site of gp130; a SHP-2 binding site of gp130; a Shc binding site of IL-2Rβ; a STAT5 binding site of IL-2Rβ; a STAT3 binding site of IL-2Rβ; a STAT1 binding site of IL-2Rβ; a STAT5 binding site of IL-7Rα; a phosphatidylinositol 3-kinase (PI3K) binding site of IL-7Rα; a STAT5 binding site of IL-12Rβ₂; a STAT4 binding site of IL-12Rβ₂; a STAT3 binding site of IL-12Rβ₂; a STAT5 binding site of IL-21R; a STAT3 binding site of IL-21R; and a STAT1 binding site of IL-21R. In certain aspects, at least one signaling molecule binding site comprises a sequence that further comprises an amino acid listed in Table 16.

In certain aspects, at least a portion of the ICDs described herein comprise the Box 1 and Box regions of gp130 or G-CSFR. In certain aspects the Box 1 region comprises a sequence of amino acids listed in Table 2. In certain aspects the Box 1 region comprises an amino acid sequence that is greater than 50% identical to a Box 1 sequence listed in Table 16.

In certain aspects, the intracellular domain of the different cytokine receptor is a wild-type intracellular domain.

In certain aspects, the chimeric variant receptors described herein further comprise at least a portion of the transmembrane domain (TMD) of a different cytokine receptor. The TMD of the different cytokine receptor can be selected from the group consisting of: gp130 (glycoprotein 130), IL-2Rβ (interleukin-2 receptor beta), IL-2Rγ or γc (IL-2 receptor gamma), IL-7Rα (interleukin-7 receptor alpha), IL-12Rβ₂ (interleukin-12 receptor beta 2) and IL-21R (interleukin-21 receptor). In certain aspects, at least a portion of the TMD comprises an amino acid sequence that shares at least 70%, at least 80%, at least 85%, at least 90%, at least 95%, at least 96%, at least 97%, at least 98%, or at least 99% amino acid identity to the amino acid sequence of a cytokine receptor TMD described herein. In certain aspects, at least a portion of a cytokine receptor TMD shares at least 70%, at least 80%, at least 85%, at least 90%, at least 95%, at least 96%, at least 97%, at least 98%, or at least 99% amino acid sequence identity to SEQ ID NO. 4, 5, 7 or 9.

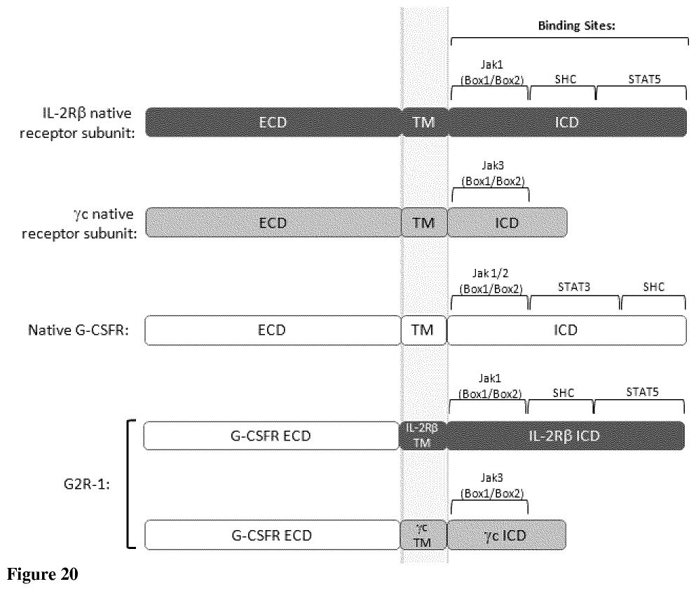

In certain aspects, the chimeric receptors described herein comprise a G-CSFR ECD domain, a transmembrane domains (TMD), and at least one portion of one ICD arranged in N-terminal to C-terminal order, as shown in a chimeric receptor design of FIGS. 20, 23 and 24.

In certain aspects, the chimeric receptor comprises the G-CSFR ECD of SEQ ID NO. 3, a portion of the gp130 TMD and ICD of SEQ ID NO 4, and a portion of the IL-2R13 ICD of SEQ ID NO. 5. In certain aspects, the chimeric receptor comprises the G-CSFR ECD of SEQ ID NO. 6, and a portion of the IL-2Rβ ICD of SEQ ID NO. 7. In certain aspects, the chimeric receptor comprises the G-CSFR ECD of SEQ ID NO. 8, and a portion of the IL-2Rγ ICD of SEQ ID NO. 9.

Binding of a variant or wild type cytokine to the chimeric cytokine receptor expressed on the surface of a cell, may or may not affect the function of the variant cytokine receptor (as compared to native cytokine receptor activity); native activity is not necessary or desired in all cases. In certain embodiments, the binding of a variant cytokine to the chimeric cytokine receptor will induce one or more aspects of native cytokine signaling. In certain embodiments, the binding of a variant cytokine to the chimeric cytokine receptor expressed on the surface of a cell causes a cellular response selected from the group consisting of proliferation, viability and enhanced activity.

Nucleic Acids Encoding Variant Cytokines and Receptors

Included in this disclosure are nucleic acids encoding any one of the receptors and variant G-CSF described herein.

A variant receptor or variant G-CSF may be produced recombinantly not only directly, but also as a fusion polypeptide with a heterologous polypeptide, e.g., a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression, the native signal sequence may be used, or other mammalian signal sequences may be suitable, such as signal sequences from secreted polypeptides of the same or related species, as well as viral secretory leaders. In certain aspects, the signal sequence can be an amino acid sequence comprising the signal sequence at the N-terminal region of SEQ ID NO. 2, 3, 6 or 8. In certain aspects, the signal sequence can be the amino acid sequence of MARLGNCSLTWAALIILLLPGSLE (SEQ ID NO. 11).

Expression Vectors Encoding Variant Cytokines or Receptors

Described herein are also expression vectors, and kits of expression vectors, which comprises one or more nucleic acid sequence(s) encoding one or more of the variant receptors or variant G-CSF described herein.

In certain embodiments, the nucleic acid encoding a variant receptor or variant G-CSF is inserted into a replicable vector for expression. Such a vector may be used to introduce the nucleic acid sequence(s) into a host cell so that it expresses a variant receptor or cytokine described herein. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like. The vector may, for example, be a plasmid or a viral vector, such as a retroviral vector, adenoviral vector, lentiviral vector, or a transposon-based vector or synthetic mRNA. The vector may be capable of transfecting or transducing a cell (e.g., a T cell, an NK cell or other cells).

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

In certain aspects, expression vectors contain a promoter that is recognized by the host organism and is operably linked to a variant protein coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells can be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp, which act on a promoter to increase its transcription. Enhancers are relatively orientation and position independent, having been found 5′ and 3′ to the transcription unit, within an intron, as well as within the coding sequence itself. Many enhancer sequences are known from mammalian genes (globin, elastase, albumin, fetoprotein, and insulin). Typically, however, one will use an enhancer from a eukaryotic cell virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors used in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

In certain aspects, disclosed herein are lentiviral vectors encoding the chimeric receptors disclosed herein. In certain aspects, the lentiviral vector comprises the HIV-1 5′ LT and a 3′ LTR. In certain aspects, the lentiviral vector comprises an EF1a promoter. In certain aspects, the lentiviral vector comprises an SV40 poly a terminator sequence. In certain aspects, the vector is psPAX2, Addgene® 12260, pCMV-VSV-G, or Addgene® 8454.

In some embodiments, also described are nucleic acid and polypeptide sequence with high sequence identity, e.g., 95, 96, 97, 98, 99% or more sequence identity to sequences described herein. The term percent sequence “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

Cells Expressing Variant Receptors and Variant Cytokines

Also described herein are cells expressing the variant receptors. Host cells, including engineered immune cells, can be transfected or transduced with the above-described expression vectors for variant cytokine or receptor expression.

In certain embodiments, the present disclosure provides a cell which comprises one or more of a variant receptor or variant cytokine described herein. The cell may comprise a nucleic acid or a vector encoding a variant receptor or variant cytokine described herein. This disclosure also provides methods of producing cells expressing a variant receptor. In certain aspects, the cells are produced by introducing into a cell the nucleic acid or expression vector described herein. The nucleic acid or expression vector can be introduced into the cell by any process including, but not limited to, transfection, transduction of a viral vector, transposition or gene editing. Any gene editing technique known in the art may be used including, but not limited to, techniques comprising clustered regularly interspaced short palindromic repeats (CRISPR-Cas) systems, zinc finger nucleases, transcription activator-like effector-based nucleases and meganucleases.

The host cell can be any cell in the body. In certain embodiments, the cell is an immune cell. In some embodiments, the cell is a T cell, including, but not limited to, naïve CD8⁺ T cells, cytotoxic CD8⁺ T cells, naïve CD4⁺ T cells, helper T cells, e.g., T_{H}1, T_{H}2, T_{H}9, T_{H}11, T_{H}22, T_{FH}; regulatory T cells, e.g., T_{R}1, natural T_{Reg}, inducible T_{Reg}; memory T cells, e.g., central memory T cells, effector memory T cells, NKT cells, γδT cells; etc. In certain embodiments, the cell is a B cell, including, but not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc. In certain embodiments, the cell is an innate lymphoid cell, including, but not limited to, NK cells, etc. In certain embodiments, the cell is a myeloid cell, including, but not limited to, macrophages, dendritic cells, myeloid-derived suppressor cells, etc.

In certain embodiments, the cell is a stem cell, including, but not limited to, hematopoietic stem cells, mesenchymal stem cells, neural stem cells, etc.

In some embodiments, the cell is genetically modified in an ex vivo procedure, prior to transfer into a subject. The cell can be provided in a unit dose for therapy, and can be allogeneic, autologous, etc. with respect to an intended recipient.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarized below.

Helper T helper cells (Th cells) assist other white blood cells in immunologic processes, including maturation of B cells into plasma cells and memory B cells, and activation of cytotoxic T cells and macrophages. Th cells express CD4 on their surface. Th cells become activated when they are presented with peptide antigens by MHC class II molecules on the surface of antigen presenting cells (APCs). These cells can differentiate into one of several subtypes, including Th1, Th2, Th3, Th17, Th9, or Tfh, which secrete different cytokines to facilitate different types of immune responses.

Cytolytic T cells (TC cells, or CTLs) destroy virally infected cells and tumor cells, and are also implicated in transplant rejection. Most CTLs express the CD8 at their surface. These cells recognize their targets by binding to antigen associated with MHC class I, which is present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress autoreactive T cells that escaped the process of negative selection in the thymus. Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD1 1c+) and plasmacytoid (CD123+) dendritic cells that have been activated with TSLP. Treg cells can be distinguished from other T cells by the presence of an intracellular molecule called FoxP3.

Adaptive Treg cells (also known as Tr1 cells or Th3 cells) may originate during a normal immune response. The cell may be a Natural Killer cell (or NK cell). NK cells form part of the innate immune system. NK cells provide rapid responses to innate signals from virally infected cells in an MHC independent manner.

In certain aspects, the cells expressing the variant receptors or variant cytokines described herein are tumor-infiltrating lymphocytes (TILs) or tumor-associated lymphocytes (TALs). In certain aspects, the TILs or TALs comprise CD4+ T cells, CD8+ T cells, Natural Killer (NK) cells, and combinations thereof.

In certain embodiments, the T cells described herein are chimeric antigen receptor T cells (CAR-T cells) that have been genetically engineered to produce an artificial T-cell receptor for use in immunotherapy. In certain aspects, the CAR-T cells derived from T cells in a patient's own blood (i.e., autologous). In certain aspects, the CAR-T are derived from the T cells of another healthy donor (i.e., allogeneic).

In certain embodiments, the T cells described herein are engineered T Cell Receptor (eTCR-T cells) that have been genetically engineered to produce a particular T Cell Receptor for use in immunotherapy. In certain aspects, the eTCR-T cells are derived from T cells in a patient's own blood (i.e., autologous). In certain aspects, the eTCR-T cells are derived from the T cells of a donor (i.e., allogeneic).

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

In certain aspects, the cells expressing chimeric cytokine receptors described herein are B cells. B cells include, but are not limited to, naïve B cells, germinal center B cells, memory B cells, cytotoxic B cells, cytokine-producing B cells, regulatory B cells (Bregs), centroblasts, centrocytes, antibody-secreting cells, plasma cells, etc.

In certain aspects, the cells expressing chimeric cytokine receptors described herein are myeloid cells, including, but not limited to, macrophages, dendritic cells, myeloid-derived suppressor cells, etc.

The cells expressing a variant receptor or variant cytokine described herein may be of any cell type. In certain aspects, the cells expressing a variant receptor or variant cytokine described herein is a cell of the hematopoietic system. Immune cells (e.g., T cells or NK cells) according to the invention may either be created ex vivo either from a patient's own peripheral blood (1st party), or in the setting of a hematopoietic stem cell transplant from donor peripheral blood (2nd party), or peripheral blood from an unconnected donor (3rd party). Alternatively, immune cells described herein may be derived from ex vivo differentiation of inducible progenitor cells or embryonic progenitor cells to immune cells. Alternatively, an immortalized immune cell line which retains its effector function (e.g., a T-cell or NK-cell line that retains its lytic function; a plasma cell line that retains its antibody producing function, or a dendritic cell line or macrophage that retains its phagocytic and antigen presentation function) and could act as a therapeutic may be used. In all these embodiments, variant receptor-expressing cells are generated by introducing DNA or RNA coding for each variant receptor(s) by one of many means including transduction with a viral vector or transfection with DNA or RNA.

The cells described herein can be immune cells derived from a subject engineered ex vivo to express a variant receptor and/or variant cytokine. The immune cell may be from a peripheral blood mononuclear cell (PBMC) sample or a tumor sample. Immune cells may be activated and/or expanded prior to being transduced with nucleic acid encoding the molecules providing the variant receptor or variant cytokine according to the first aspect of the invention, for example by treatment with an anti-CD3 monoclonal antibody and/or IL-2. The immune cell of the invention may be made by: (i) isolation of an immune cell-containing sample from a subject or other sources listed above; and (ii) transduction or transfection of the immune cells with one or more nucleic acid sequence(s) encoding a variant receptor or variant cytokine.

Cells can be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

The immune cells may then by purified, for example, selected on the basis of expression of the antigen-binding domain of the antigen-binding polypeptide. In certain embodiments, the cells are selected by expression of a selectable marker (e.g., a protein, a fluorescent marker, or an epitope tag) or by any method known in the art for selection, isolation and/or purification of the cells.

Kits

This disclosure also describes kits for producing a cell expressing at least one of any of the variant receptors or variant G-CSF described herein. In certain embodiments, the kits comprise at least one expression vector encoding at least one variant receptor and instructions for use. In certain aspects, the kit further comprises at least one variant cytokine in a pharmaceutical formulation or an expression vector encoding a variant G-CSF that binds to at least one of the variant receptors described herein. In certain embodiments, the kits comprise a cell comprising an expression vector encoding a variant receptor described herein.

In certain embodiments, the kits comprise a cell comprising an expression vector encoding a Chimeric Antigen Receptor (CAR)/T cell receptor (TCR) or the like. (CAR)/engineered T cell receptor (eTCR) or the like (e.g., engineered non-native TCR receptors). In certain embodiments the kits comprise an expression vector encoding a Chimeric Antigen Receptor (CAR)/engineered T cell receptor (eTCR) or the like. In certain embodiments the kits comprise an expression vector encoding a variant receptor described herein and a Chimeric Antigen Receptor (CAR)/engineered T cell receptor (eTCR) or the like.

In certain aspects, the kits described herein further comprise a variant cytokine. In certain embodiments, the kit further comprises at least one additional variant? cytokine. In certain aspects, the kits further comprise at least one variant cytokine in a pharmaceutical formulation. In certain embodiments, the components are provided in a dosage form, in liquid or solid form in any convenient packaging.

Additional reagents may be provided for the growth, selection and preparation of the cells provided or cells produced as described herein. For example, the kit can include components for cell culture, growth factors, differentiation agents, reagents for transfection or transduction, etc.

In certain embodiments, in additional to the above components, the kits may also include instructions for use. Instructions can be provided in any convenient form. For example, the instructions may be provided as printed information, in the packaging of the kit, in a package insert, etc. The instructions can also be provided as a computer readable medium on which the information has been recorded. In addition, the instructions may be provided on a website address which can be used to access the information.

Methods of Selective Activation of a Variant Receptor

This disclosure provides methods for selective activation of a variant receptor expressed on the surface of a cell, comprising contacting the variant receptor described herein with a cytokine that selectively activates the chimeric receptor. In certain aspects, the cytokine that selectively activates the chimeric receptor is a variant G-CSF. The G-CSF can be a wild-type G-CSF or a G-CSF comprising one or more mutations that confer preferential binding and activation of the G-CSF to a variant receptor compared to the native (wild-type) cytokine receptor.

In certain aspects, the selective activation of the variant receptor by binding of the cytokine to the variant receptor leads to homodimerization, heterodimerization, or combinations thereof.

In certain aspects, activation of the variant receptor leads to activation of downstream signaling molecules. In certain aspects, the variant receptor activates signaling molecules or pathways that are transduced through native cellular signaling molecules to provide for a biological activity that mimics that native response, but which is specific to a cell engineered to express the variant receptor. In certain aspects, the activation of the downstream signaling molecules includes activation of cellular signaling pathways that stimulate cell cycle progression, proliferation, viability, and/or enhanced activity. In certain aspects, the signaling pathways or molecules that are activated are, but not limited to, Jak1, Jak2, Jak3, STAT1, STAT2, STAT3, STAT4, STAT5A, STAT5B, STAT6, Shc, ERK1/2 and Akt. In certain aspects, activation of the variant receptor leads to increased proliferation of the cell after administration of a cytokine that binds the receptor. In certain aspects, the extent of proliferation is between 0.1-10-fold the proliferation observed when the cells are stimulated with IL-2.

Methods of Adoptive Cell Transfer

The present invention provides a method for treating and/or preventing a disease which comprises the step of administering the cells expressing a variant receptor and/or a variant cytokine described herein (for example in a pharmaceutical composition as described below) to a subject.

A method for treating a disease relates to the therapeutic use of the cells described herein, e.g., T cells, NK cells, or any other immune or non-immune cells expressing a variant receptor. The cells can be administered to a subject having an existing disease or condition in order to lessen, reduce or improve at least one symptom associated with the disease and/or to slow down, reduce or block the progression of the disease. The method for preventing a disease relates to the prophylactic use of the cells of the present disclosure. Such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

In some embodiments, the subject compositions, methods and kits are used to enhance an immune response. In some embodiments the immune response is directed towards a condition where it is desirable to deplete or regulate target cells, e.g., cancer cells, infected cells, immune cells involved in autoimmune disease, etc. by systemic administration of cytokine, e.g. intramuscular, intraperitoneal, intravenous, and the like.

The method can involve the steps of: (i) isolating an immune cell-containing sample; (ii) transducing or transfecting such cells with a nucleic acid sequence or vector e.g., expressing a variant receptor; (iii) administering (i.e., infusing) the cells from (ii) to the subject, and (iv) administering a variant cytokine that stimulates the infused cells. In certain aspects, the subject has undergone an immuno-depletion treatment prior to administering the cells to the subject. In certain aspects, the subject has not undergone an immuno-depletion treatment prior to administering the cells to the subject. In certain aspects, the subject has undergone an immuno-depletion treatment reduced in severity, dose and/or duration that would otherwise be necessary without the use of the variant receptors described herein prior to administering the cells to the subject.

The immune cell-containing sample can be isolated from a subject or from other sources, for example as described above. The immune cells can be isolated from a subject's own peripheral blood (1st party), or in the setting of a hematopoietic stem cell transplant from donor peripheral blood (2nd party), or peripheral blood from an unconnected donor (3rd party). The immune cells can also be derived by in vitro methods, such as induced differentiation from stem cells or other forms of precursor cell.

In some embodiments, the immune cells are contacted with the variant cytokine in vivo, i.e., where the immune cells are transferred to a recipient, and an effective dose of the variant cytokine is administered to the recipient and allowed to contact the immune cells in their native location, e.g. in lymph nodes, etc. In some embodiments, the contacting is performed in vitro. Where the cells are contacted with the variant cytokine in vitro, the cytokine is added to the cells in a dose and for a period of time sufficient to activate signaling from the receptor, which can utilize aspects of the native cellular machinery, e.g. accessory proteins, co-receptors, etc. The activated cells can be used for any purpose, including, but not limited to, experimental purposes relating to determination of antigen specificity, cytokine profiling, and for delivery in vivo.

In certain aspects, a therapeutically effective number of cells are administered to the subject. In certain aspects, the subject is administered or infused with cells expressing variant receptors on a plurality of separate occasions. In certain embodiments, at least 1×10⁶ cells/kg, at least 1×10⁷ cells/kg, at least 1×10⁸ cells/kg, at least 1×10⁹ cells/kg, at least 1×10¹⁰ cells/kg, or more are administered, sometimes being limited by the number of cells, e.g., transfected T cells, obtained during collection. The transfected cells may be infused to the subject in any physiologically acceptable medium, normally intravascularly, although they may also be introduced into any other convenient site, where the cells may find an appropriate site for growth.

In certain aspects, a therapeutically effective amount of variant cytokine is administered to the subject. In certain aspects, the subject is administered the variant cytokine on a plurality of separate occasions. In certain aspects, the amount of variant cytokine that is administered, is an amount sufficient to achieve a therapeutically desired result (e.g., reduce symptoms of a disease in a subject). In certain aspects, the amount of variant cytokine that is administered is an amount sufficient to stimulate cell cycle progression, proliferation, viability and/or functional activity of a cell expressing a variant cytokine receptor described herein. In certain aspects, the variant cytokine is administered at a dose and/or duration that would is necessary to achieve a therapeutically desired result. In certain aspects, the variant cytokine is administered at a dose and/or duration sufficient to stimulate cell cycle progression, proliferation, viability and/or functional activity of a cell expressing a variant cytokine receptor described herein. Dosage and frequency may vary depending on the agent; mode of administration; nature of the cytokine; and the like. It will be understood by one of skill in the art that such guidelines will be adjusted for the individual circumstances. The dosage may also be varied for localized administration, e.g. intranasal, inhalation, etc., for systemic administration, e.g., intramuscular, intraperitoneal, intravascular, and the like.

Indications for Adoptive Cell Transfer

The present disclosure provides a cell expressing a variant receptor described herein for use in treating and/or preventing a disease. The invention also relates to the use of a cell expressing a variant receptor described herein in the manufacture of a medicament for the treatment and/or prevention of a disease.

The disease to be treated and/or prevented by the methods of the present invention can be a cancerous disease, such as, but not limited to, bile duct cancer, bladder cancer, breast cancer, cervical cancer, ovarian cancer, colon cancer, endometrial cancer, hematologic malignancies, kidney cancer (renal cell), leukemia, lymphoma, lung cancer, melanoma, non-Hodgkin lymphoma, pancreatic cancer, prostate cancer, sarcoma and thyroid cancer.

The disease to be treated and/or prevented can be an autoimmune disease. Autoimmune diseases are characterized by T and B lymphocytes that aberrantly target self-proteins, polypeptides, peptides, and/or other self-molecules causing injury and or malfunction of an organ, tissue, or cell-type within the body (for example, pancreas, brain, thyroid or gastrointestinal tract) to cause the clinical manifestations of the disease. Autoimmune diseases include diseases that affect specific tissues as well as diseases that can affect multiple tissues, which can depend, in part on whether the responses are directed to an antigen confined to a particular tissue or to an antigen that is widely distributed in the body. Autoimmune diseases include, but are not limited to, Type 1 diabetes, systemic lupus erythematosus, Rheumatoid arthritis, autoimmune thyroid diseases and Graves' disease.

The disease to be treated and/or prevented can be an inflammatory condition, such as cardiac fibrosis. In general, inflammatory conditions or disorders typically result in the immune system attacking the body's own cells or tissues and may cause abnormal inflammation, which can result in chronic pain, redness, swelling, stiffness, and damage to normal tissues. Inflammatory conditions are characterized by or caused by inflammation and include, but are not limited to, celiac disease, vasculitis, lupus, chronic obstructive pulmonary disease (COPD), irritable bowel disease, atherosclerosis, arthritis, myositis, scleroderma, gout, Sjorgren's syndrome, ankylosing spondylitis, antiphospholipid antibody syndrome, and psoriasis.

In certain embodiments, the method is used to treat an infectious disease.

In certain embodiments, the condition to be treated is to prevent and treat graft rejection. In certain embodiments, the condition to be treated and/or prevented is allograft rejection. In certain aspects, the allograft rejection is acute allograft rejection.

The disease to be treated and/or prevented can involve the transplantation of cells, tissues, organs or other anatomical structures to an affected individual. The cells, tissues, organs or other anatomical structures can be from the same individual (autologous or “auto” transplantation) or from a different individual (allogeneic or “allo” transplantation). The cells, tissues, organs or other anatomical structures can also be produced using in vitro methods, including cell cloning, induced cell differentiation, or fabrication with synthetic biomaterials.

The present invention provides a method for treating and/or preventing a disease which comprises one or more steps of administering the variant cytokine and/or cells described herein (for example in a pharmaceutical composition as described above) to a subject.

A method for treating and/or preventing a disease relates to the therapeutic use of the cells of the present disclosure. Herein the cells may be administered to a subject having an existing disease or condition in order to lessen, reduce or improve at least one symptom associated with the disease and/or to slow down, reduce or block the progression of the disease. The method for preventing a disease relates to the prophylactic use of the cells of the present disclosure. Such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease. The method may involve the steps of: (i) isolating an immune cell-containing sample; (ii) transducing or transfecting such cells with a nucleic acid sequence or vector provided by the present invention; (iii) administering the cells from (ii) to a subject, and (iv) administering a variant cytokine that stimulates the infused cells. The immune cell-containing sample may be isolated from a subject or from other sources, for example as described above. The immune cells may be isolated from a subject's own peripheral blood (1st party), or in the setting of a hematopoietic stem cell transplant from donor peripheral blood (2nd party), or peripheral blood from an unconnected donor (3rd party).

Treatment can be combined with other active agents, such as, but not limited to, antibiotics, anti-cancer agents, anti-viral agents, and other immune modulating agents (e.g., antibodies against the Programmed Cell Death Protein-1 [PD-1] pathway or antibodies against CTLA-4). Additional cytokines may also be included (e.g., interferon γ, tumor necrosis factor α, interleukin 12, etc.).

### Methods Using Stem Cells Expressing Variant Cytokine Receptors

The present invention provides a method for treating and/or preventing a condition or disease which comprises the step of administering stem cells expressing a variant receptor and/or a variant cytokine described herein. In certain embodiments, stem cells expressing the variant cytokine receptors and/or variant cytokines described herein are used for regenerative medicine, cell/tissue/organ transplantation, tissue reconstruction, or tissue repair.

### Pharmaceutical Compositions of the Invention

The present disclosure also relates to a pharmaceutical composition containing a plurality of cells expressing a variant receptor described herein and/or the cytokines described herein. The present disclosure also relates to a pharmaceutical composition containing a variant cytokine described herein. The cells of the invention can be formulated in pharmaceutical compositions. These compositions can comprise, in addition to one or more of the cells expressing the variant receptor described herein, a pharmaceutically acceptable excipient, carrier, buffer, stabilizer or other materials well known to those skilled in the art. Such materials should be non-toxic and should not interfere with the efficacy of the active ingredient. The pharmaceutical composition may optionally comprise one or more further pharmaceutically active polypeptides and/or compounds. Such a formulation may, for example, be in a form suitable for intravenous infusion.

For cells expressing a variant receptor, and variant cytokines described herein, according to the present disclosure that are to be given to an individual, administration is preferably in a “therapeutically effective amount” that is sufficient to show benefit to the individual. A “prophylactically effective amount” can also be administered, when sufficient to show benefit to the individual. The actual amount of cytokine or number of cells administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A pharmaceutical composition can be administered alone or in combination with other treatments, either simultaneously or sequentially dependent upon the condition to be treated.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques, cell culture, adoptive cell transfer, and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992).

### Example 1: Rational Design of an Exclusive Site II Interface of G-CSF:G-CSFR(CRH)

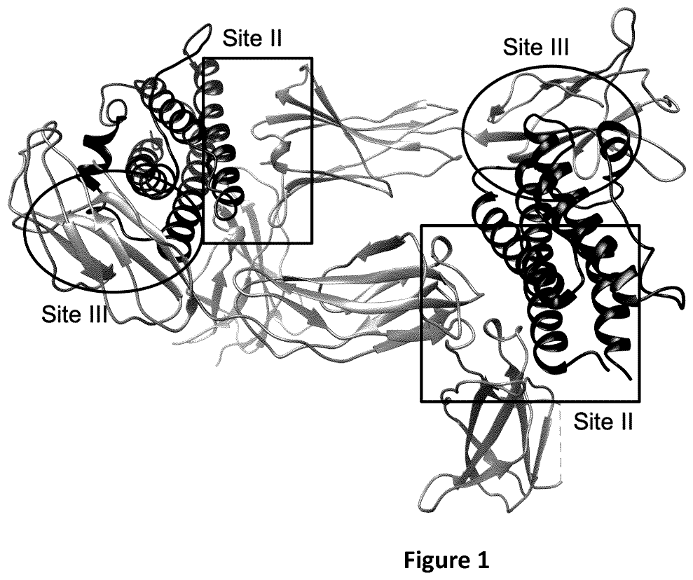

The wild type (WT) G-CSF_{WT}:G-CSFR^{WT} complex is a 2:2 heterodimer. G-CSF has two binding interfaces with the extracellular domain (ECD) of G-CSFR. The larger interface between G-CSF and the extracellular Cytokine Receptor Homologous (CRH) domain of G-CSFR is referred to as site II. The smaller interface between G-CSF and the N-terminal Ig-like (Ig) extracellular domain of G-CSFR is referred to as site III (see, FIG. 1).

To design co-evolved, engineered (E) G-CSF_{E}:G-CSFR_{E} cytokine: receptor pairs, we separated the 2:2 complex between WT G-CSF and the Ig-CRH extracellular domain of G-CSFR (Protein data Bank ID 2D9Q, Tamada et al. PNAS 2006) into the two distinct sub-complexes encompassing the site II and site III interface, consisting of G-CSF:G-CSFR(CRH) and G-CSF:G-CSFR(Ig) (see, FIG. 1), respectively, see Table 1 for sequences.

Methods

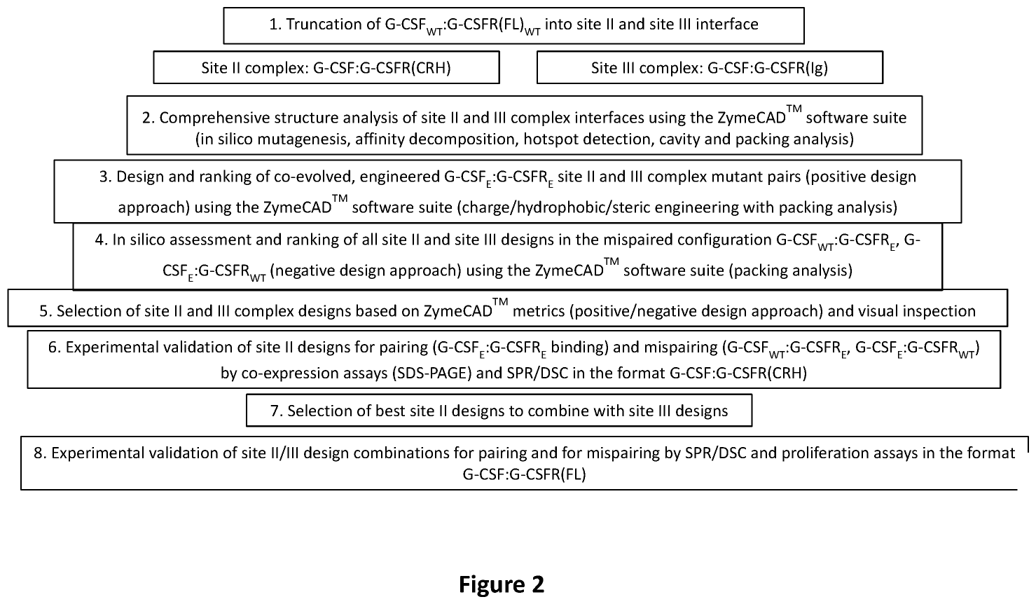

To create co-evolved, exclusive G-CSF_{E}:G-CSFR_{E} mutant pairs (designs), we employed a computational design workflow (see FIG. 2). First, we created exclusive designs at site II.

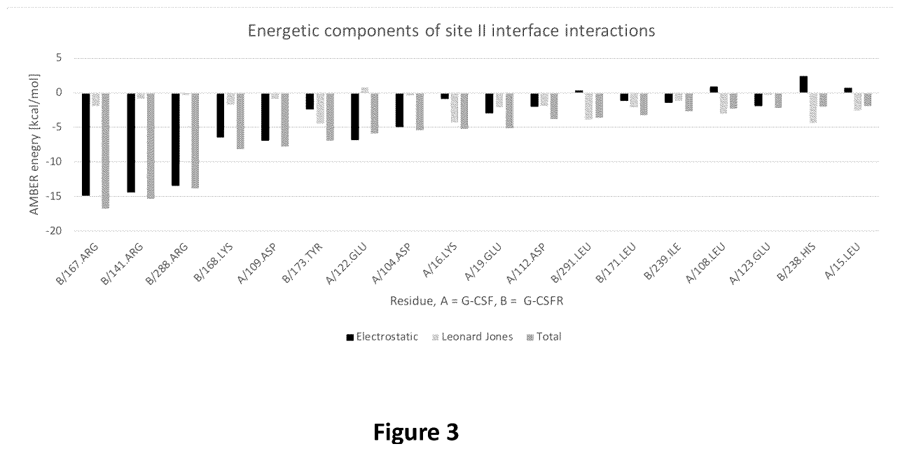

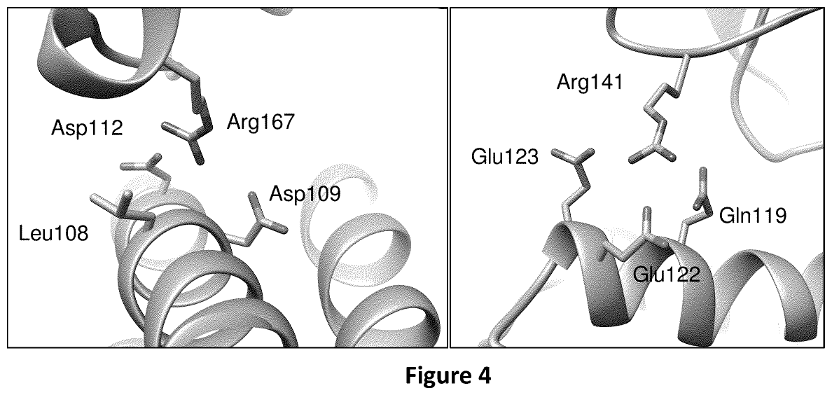

In silico structural analysis of site II interface interactions of G-CSF_{WT}:G-CSFR(CRH)_{WT} showed that most molecular interactions, that contribute to the total attractive AMBER energy at site II of 109.64 kcal/mol, are made by charged residues (see, FIG. 3). In depth inspection reveals mostly electrostatic and hydrogen-bonding interactions, for example between R167 of G-CSFR(CRH) and D112/D109 of G-CSF, which contributes 28% of the total attractive AMBER energy at site II. Another important electrostatic interaction is present between R141 of G-CSFR(CRH) and E122/E123 of G-CSF (see, FIG. 4), which contributes 21.4% of the total attractive AMBER energy at site II (See FIG. 3). Likewise, the salt bridge between E19 of the cytokine and R288 of the receptor CRH domain contributes 17.3%, the arginine is further stabilized through electrostatic and hydrogen-bonding interaction to D200 of the receptor CRH domain.

Each design at site II consists of a mutant pair of G-CSF_{E} and G-CSFR(CRH)_{E}. First, we created positive designs at site II, for example through inverting charges, by mutating basic residues to acidic residues and vice versa on the binding partner, while maintaining packing and hydrophobic interactions through additional mutations if necessary. Mutant pairs G-CSF_{E}:G-CSFR(CRH)_{E} were packed with the mean-field packing workflow of ZymeCAD™. The packed in silico models of designs at site II were visually inspected for structural integrity and they were assessed by ZymeCAD™ metrics. Specifically, we aimed to design G-CSF_{E} mutants to have ZymeCAD™ in silico dAMBER binding affinity of <10 kcal/mol for their corresponding G-CSFR(CRH)_{E} mutant (paired interaction). This metric compares AMBER affinity, the sum of Lennard Jones affinity and Electrostatics affinity, to AMBER affinity of the WT:WT cytokine-receptor pair. Designs with dAMBER_folding >80 kcal/mol were excluded. The dAMBER folding metric scores the change in the sum of Lennard Jones-bonded folding and Electrostatics folding upon mutation. We also excluded designs that scored higher than 400 kcal/mol in dDDRW apostability. This metric describes the change in knowledge-based potential stability upon mutation of a protein from its apo form.

Next we packed G-CSF_{E} mutants of each design in a complex with WT G-CSFR (and vice versa G-CSFR_{E} with G-CSF_{WT}) with ZymeCAD™, to assess the metrics of each positive design under conditions of mispairing when the following two complexes would form: G-CSF_{WT}:G-CSFR(CRH)_{E} and G-CSF_{E}:G-CSFR(CRH)_{WT}. We calculated in silico ddAMBER metrics for the mispaired orientations with ZymeCAD™ (DdAMBER_affinity_Awt_Bmut is the AMBER affinity of the paired, engineered complex subtracted by the AMBER affinity of the mispaired complex G-CSF_{WT}:G-CSFR(CRH)_{E}, ddAMBER_affinity_Amut_Bwt is the AMBER affinity of the paired, engineered complex subtracted by the AMBER affinity of the mispaired complex G-CSF_{E}:G-CSFR(CRH)_{WT}. Designs that minimized mispaired ddAMBER affinity metrics were considered to be more selective for their paired binding partner over binding to wild type cytokine or receptor binding.

All site II designs were clustered and the packing metrics of G-CSF_{E}:G-CSFR(CRH)_{E}, G-CSF_{WT}:G-CSFR(CRH)_{E}, G-CSF_{E}:G-CSFR(CRH)_{WT} were considered to assess the strength of pairing (positive design) and selectivity against mispairing with WT G-CSF and G-CSFR(CRH) (negative design) and rank designs.

Results

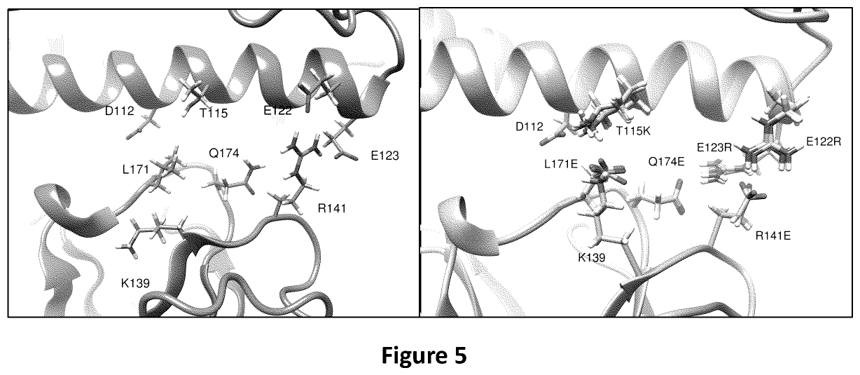

Designs listed in Table 2 have packing metrics in ZymeCAD™ that favour pairing of G-CSF_{E}:G-CSFR(CRH)_{E} in silico (see, Table 3) and showed satisfying interactions in silico in the visual inspection such as the presence of salt bridges, hydrogen bonds and absence of severe clashes (see, FIG. 5). These designs also showed high selectivity against mispairing with WT G-CSF or G-CSFR(CRH) (see, Table 3).

Therefore, the site II mutations of the same variant G-CSF and receptor designs shown in Table 2 are predicted to exhibit preferential binding, over wild type G-CSFR and G-CSF, respectively.

TABLE 3
Site II designs with AMBER metrics in kcal/mol from
triplicate in silico mean-field packs with ZymeCAD^{Tm}.

| | G-CSF_{E}:G-CSFR(CRH)_{E} | | G-CSF_{E}:G-CSFR(CRH)_{WT} | G-CSF_{WT}:G-CSFR(CRH)_{E} |
|---|---|---|---|---|
| Design | dAMBER_aff | dAMBER_folding | ddAMBER_aff_E_WT | ddAMBER_aff_WT_E |
| 1 | −13.2 | 36.0 | −35.2 | −47.3 |
| 2 | −24.2 | 35.6 | −41.0 | −55.8 |
| 6 | 8.9 | −7.5 | −8.8 | −13.3 |
| 7 | −24.1 | −7.8 | −45.8 | −51.7 |
| 8 | −30.5 | 12.2 | −73.5 | −86.5 |
| 9 | −25.1 | 17.4 | −89.6 | −119.5 |
| 15 | −53.3 | 59.5 | −72.4 | −71.0 |
| 17 | −0.2 | 19.3 | −23.2 | −28.5 |
| 18 | −17.6 | 75.2 | −44.2 | −27.8 |
| 19 | 20.6 | 38.1 | −10.7 | −17.3 |
| 20 | −9.9 | 28.5 | −52.7 | −64.4 |
| 21 | −18.0 | 51.5 | −55.4 | −47.8 |
| 22 | −32.3 | 52.2 | −75.0 | −53.3 |
| 24 | −13.0 | 57.7 | −50.8 | −86.4 |
| 25 | −49.8 | 83.5 | −100.2 | −91.3 |
| 27 | −60.5 | 43.0 | −131.4 | −156.4 |
| 28 | −61.6 | 82.6 | −148.8 | −135.9 |
| 30 | 9.6 | −5.0 | −31.4 | −29.1 |
| 34 | 2.9 | 16.7 | −60.4 | −63.1 |
| 35 | −27.4 | 0.3 | −50.1 | −63.0 |
| 36 | −92.2 | 35.1 | −110.7 | −93.5 |
| 42 | −82.1 | 67.7 | −137.0 | −119.7 |
| 43 | −14.9 | 51.0 | −87.4 | −111.2 |
| 44 | −20.2 | 53.6 | −86.4 | −108.4 |
| 45 | −35.5 | 38.1 | −70.7 | −107.0 |
| 46 | −11.6 | 44.2 | −75.0 | −95.8 |
| 47 | −27.8 | 75.8 | −72.4 | −91.5 |
| 48 | −69.7 | 76.8 | −127.8 | −87.7 |
| 49 | −55.9 | 79.3 | −94.7 | −84.7 |
| 50 | −52.0 | 38.6 | −123.5 | −134.9 |
| 80 | 15.3 | 13.0 | −16.2 | 10.8 |
| 81 | −2.4 | 52.7 | −24.5 | −28.9 |
| 82 | 29.8 | 10.9 | 1.6 | 0.7 |
| 83 | 36.3 | 54.6 | −21.6 | 5.5 |

### Example 2: In Vitro Screening of Site II Designs

Selected site II designs were screened in a pulldown experiment in the format G-CSF_{E}:G-CSFR(CRH)_{E} for their ability to form a site II complex when co-expressed in a Baculovirus based insect cell system. We also assessed whether designs could form a mispaired complex with WT receptor or cytokine by co-expressing each designs G-CSF_{E} mutant with G-CSFR(CRH)_{WT}, and vice versa each design G-CSFR(CRH)_{E} mutant was co-expressed with GCSF_{WT}. The expression of G-CSF_{E} alone was verified by single infections of the cytokine mutant.

Methods

In brief, site II G-CSF designs and corresponding, paired G-CSFR(CRH) mutants were cloned separately into insect cell transfection vectors. G-CSF WT (residues 1-173, Table 1) and mutants were cloned into a modified pAcGP67b transfer vector (Pharmingen), in frame with an N-terminal secretion signal and a C-terminal TEV-cleavable Twin Strep tag with the sequence AAAENLYFQ/GSAWSHPQFEKGGGSGGGSGGSAWSHPQFEK (SEQ ID NO. 82). Of the receptor extracellular domain, only the CRH domain (residues 98-308, Table 1) with site II design mutations was cloned into a modified pAcGP67b transfer vector, in frame, with an N-terminal secretion signal and TEV-cleavable Hexahistidine (SEQ ID NO. 89) tag of the sequence HHHHHHSSGRENLYFQ/GSMG (SEQ ID NO. 83). All constructs were synthesized and codon optimized for insect cell expression (Genscript). Transfer vector DNA was prepared by Midi-prep (ThermoScientific, cat. K0481), was endotoxin-free and had a A260/280 absorbance ratio of 1.8-2.0. Recombinant virus generation was achieved by co-transfection of recombinant, linearized Baculovirus DNA with the vector DNA in Spodoptera frugiperda 9 (Sf9) cells, using the adherent method, as described by the manufacturer (Expression Systems, California). Approximately 1 h prior to transfection, 2 mL of healthy, log-phase Sf9 cells at 0.46×10⁶ cells ml⁻¹ were seeded per well of a 6-well tissue culture plate (Greiner, cat. 657-160). Transfection mixtures were prepared as follows: 100 μl Transfection Medium (Expression Systems, California, cat. 95-020-100) was placed into each of two sterile 1.5 ml microfuge tubes, A and B. To Tube A, 0.4 μg recombinant BestBac 2.0 Δ v-cath/chiA linearized DNA (Expression Systems, California, cat. 91-002) and 2 μg vector DNA was added. To Tube B, 1.2 μl 5× Express²TR transfection reagent (Expres2ION, cat. 52-55A-001) was added. Solutions A and B were incubated at approximately 24° C. for 5 minutes and then combined and incubated for 30 minutes. After incubation 800 μl Transfection Medium was added to each transfection reaction to increase the volume to 1 ml. The old ESF 921 medium was removed from the wells and replaced with 1 mL transfection mixture applied drop-by-drop so as not to disturb the cell monolayer. Plate(s) were gently rocked back-and-forth and from side-to-side to evenly distribute the transfection mixtures, and incubated at 27° C. for 4 h. After 4 h, the transfection mixture was removed from the co-transfection plate(s) and 2 mL fresh ESF 921 insect cell culture medium (Expression Systems, California, cat. 96-001-01) containing gentamicin at 10 μg/ml (cat. 15750-060) was added drop-by-drop. To prevent evaporation, plates were wrapped in saran wrap, placed in a sterile plastic box and incubated for 4-5 days at 27° C. At day 4 or 5 post-transfection P1 supernatants were collected and clarified by centrifugation at 5000 rpm for 5 minutes and transferred to a new sterile tube and stored at 4° C., protected from light.

The recombinant P1 stocks produced as described above were further amplified to high-titre, low passage P2 stocks for protein expression studies. The following workflow produced 50-100 mL virus using the P1 seed stock of virus harvested from the co-transfection as inoculum. 50 mL of log-phase Sf9 cells at 1.5×10⁶ cells ml⁻¹ were seeded into a 250 mL shake flask (FisherScientific, cat. PBV 250), and 0.5 mL P1 virus stock was added. Cells were incubated at 27° C., with shaking at 135 rpm and monitored for infection. P2 virus supernatant was harvested 5-7 days post infection and clarified by centrifugation at 4000 rpm for 10 minutes. To minimize titre loss, 10% heat-inactivated FBS (VWR, cat. 97068-085) was added and P2 virus stored at 4° C. in the dark.

The P2 viral stocks were tested for protein expression in small-scale. P2 virus was used to co-infect G-CSF_{E} mutants with their corresponding G-CSFR_{E} mutant in Trichoplusia ni (Tni) cells in 12-well plates. Separate co-infections for each design mutant were performed using the P2 stocks of G-CSF_{WT} and GCSFR(CRH)_{WT}, as well as for G-CSF_{E} mutants alone. For each reaction, 2 mL of healthy log-phase Tni at 2×10⁶ cells ml⁻¹ was inoculated with 20 μl P2 virus. Plates were incubated at 27° C. for approximately 70 h with shaking at 135 rpm. Supernatants were clarified by centrifugation at 5000 rpm for 3 minutes and secreted protein was pulled down, via the Twin-Strep Tag (TST) on G-CSF_{E} mutants and G-CSF_{WT}, in batch mode with streptactin-XT superflow (IBA Lifesciences, cat. 2-4010-010). Briefly, to each 1.8 mL reaction supernatant 0.2 mL 10×HEPES Buffered Saline (HBS: 20 mM HEPES pH8, 150 mM NaCl) was added to 1×, 20 μl bed volume (b.v) of purification beads added and reactions incubated for 30 minutes with end-over-end mixing at 24° C. An additional 20 μl b.v of purification beads was added followed by a second 30 minute incubation. Beads were pelleted by centrifugation at 2200 rpm for 3 minutes, supernatant was removed and the beads washed with 1×HBS buffer. Protein was eluted with 30 μl BXT elution buffer (100 mM Tris-CL pH8, 150 mM NaCl, 1 mM EDTA, 50 mM Biotin, (IBA Lifesciences, cat. 2-1042-025)), boiled with SDS-PAGE sample buffer and analyzed on a 12% Bolt Bis-Tris plus, 12 well gel (Thermo Fisher Scientific, cat. NW00122BOX) run at 200V for 30 minutes under reducing conditions.

Results

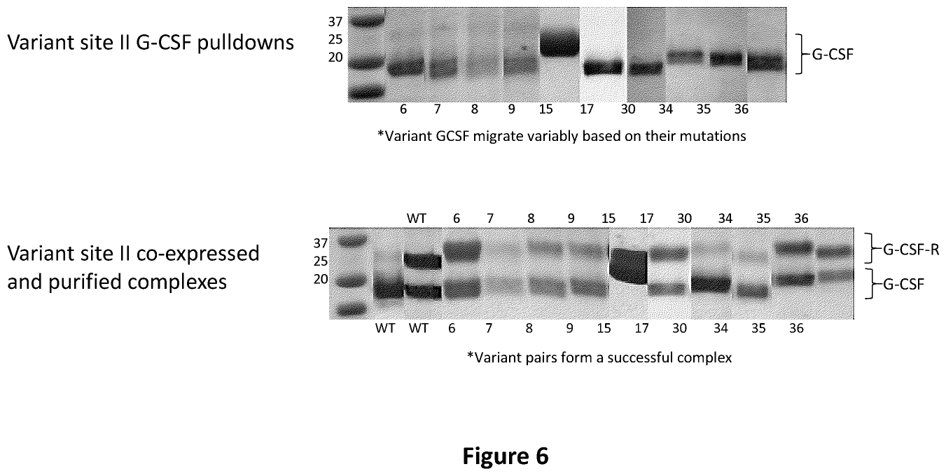

Site II designs #6, 7, 8, 9, 15, 17, 30, 34, 35, 36 showed expression for G-CSF_{E} alone, and formed sufficiently stable, paired G-CSF_{E}:G-CSFR_{E} complexes that were pulled down through the Twin Strep tag on G-CSF_{E} (see, FIG. 6). For example, site II design #6 post pulldown of the engineered complex showed a band on SDS-PAGE for its G-CSF_{E} mutant at ˜22 kDa as well as for its corresponding, co-expressed G-CSFR(CRH)_{E} mutant at ˜33 kDa (see, FIG. 6).

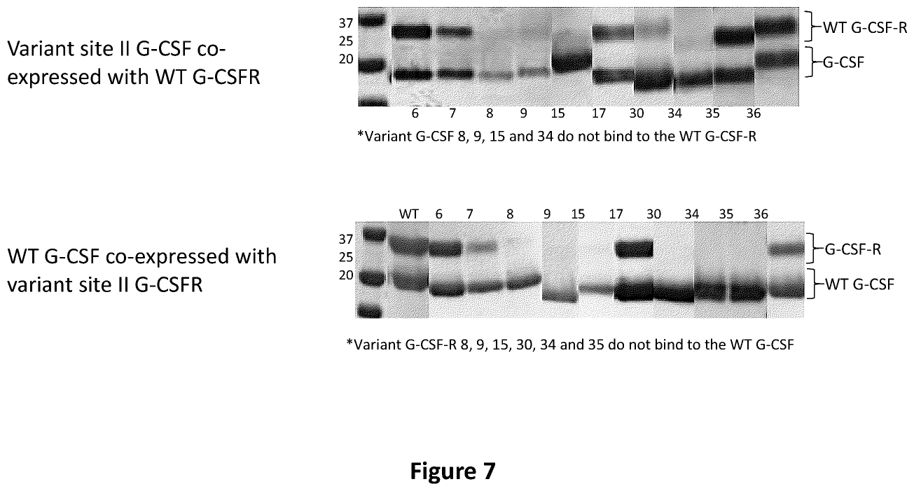

Site II designs #8, 9, 15 and 34 were also selective against mispairing with WT G-CSF and WT G-CSFR (see, FIG. 7) in the co-expression assay because they were not pulled down by WT G-CSF (see, FIG. 7 bottom panel), and WT receptor was not pulled down by the G-CSF_{E} mutant (see, FIG. 7 top panel). Site II G-CSFR_{E} design 30 and 35 were selective against mispairing with the WT G-CSF in the co-expression assay because they were not pulled down by WT G-CSF (see, FIG. 7 bottom panel), however, the reciprocal G-CSF_{E} design was not selective against mispairing with the WT G-CSFR in co-expression assay because WT G-CSFR was pulled down (see, FIG. 7 top panel). Site II designs 6, 7, 17 and 36 were not selective against mispairing with the WT G-CSF or WT G-CSFR (see, FIG. 7) in the co-expression assay because they pulled down WT G-CSFR and were pulled down by WT G-CSF.

Site II receptor mutants with mutations at residue R288 did not express in the G-CSFR(CRH) receptor chain format without Ig domain. R288 containing designs were evaluated for paired complex formation by SPR in combination with site III designs on the Ig domain (see Example 6). Therefore, several site II designs were identified that formed sufficiently stable, paired G-CSF_{E}:G-CSFR_{E} complexes that were also selective against mispairing with WT G-CSF and WT G-CSFR.

Therefore, various site II mutations of the same variant G-CSF and receptor designs shown in Table 2 exhibit preferential binding over wild type G-CSFR and G-CSF, respectively.

### Example 3: Rational Design of an Exclusive Site III Interface of G-CSF:G-CSFR(Ig)

The avidity effect of the site III receptor Ig domain binding to G-CSF contributes to the formation of the 2:2 heterodimer stoichiometry of the G-CSF:G-CSFR(Ig-CRH) complex (see, FIG. 1). A selective design present only at site II with a WT site III interface appears to be insufficient to create a fully exclusive 2:2 G-CSF_{E}:G-CSFR(Ig-CRH)_{E} complex. To favor binding of a paired, co-evolved design to be selective over mispaired binding to WT cytokine or receptor, we applied the in silico design workflow described in Example 1 to the site III interface to create selective site III designs (see FIG. 2).

Methods

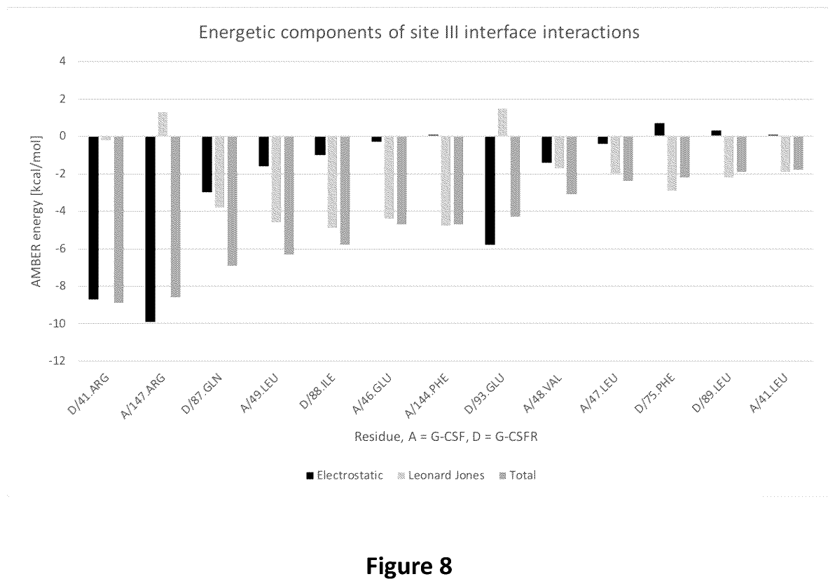

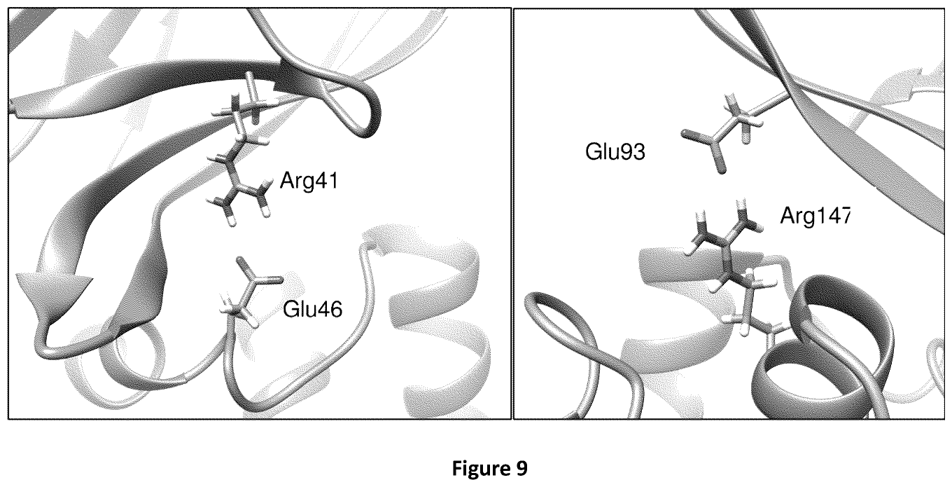

First, we conducted a structural analysis of site III interface interactions. The site III interface contributes 55.64 kcal/mol AMBER energy to the G-CSF:G-CSFR complex and it has an overall smaller interface area with 571.5 Å² compared to site II with an interface area of 692.6 Å². In depth inspection of site III interface reveals less electrostatic and hydrogen-bonding interactions compared to the site II interface (see, FIG. 8). Key interactions at site III are for example a salt bridge between E46 of G-CSF and R41 of the receptor Ig domain, furthermore between R147 of G-CSF and E93 of the receptor Ig domain (see, FIG. 9). Both interactions contribute 15.9% and 15.4%, respectively, to the total attractive AMBER energy of site III. Further interactions are for example made through Q87 of the receptor Ig domain, which forms a hydrogen-bonding interaction with its side chain amide to the backbone of G-CSF site III, and has Lennard Jones interactions with surrounding side chains of the cytokine such as E46 and L49. This interaction makes up 12.3% of the total attractive AMBER energy at site III.

Next, we created positive designs at site III where G-CSF_{E} mutant has a favorable AMBER binding affinity in silico for its co-evolved G-CSFR(Ig)_{E} mutant (paired interaction). This was done for example through inverting charges or changing shape complementarity while maintaining favorable Lennard Jones and hydrogen bonding interactions. Mutant pairs G-CSF_{E}:G-CSFR(Ig)_{E} were packed with the mean-field packing workflow of ZymeCAD™. The packed in silico models of designs at site III were visually inspected for structural integrity and they were assessed by ZymeCAD™ metrics as described in Example 1. Next, we packed G-CSF_{E} mutants of each design with WT G-CSFR(Ig) (and vice versa G-CSFR(Ig)_{E} with G-CSF_{WT}) with ZymeCAD™ to assess the metrics under conditions of mispairing with WT cytokine and receptor. We calculated ddAMBER affinity metrics (G-CSF_{WT}:G-CSFR(Ig)_{E}, G-CSF_{E}:G-CSFR(Ig)_{WT}) for site III designs with ZymeCAD™ (see Table 5) as described before for site II in Example 1.

Site III designs were clustered and the packing metrics of all three in silico complexes G-CSF_{E}:G-CSFR(Ig)_{E}, G-CSF_{WT}:G-CSFR(Ig)_{E}, G-CSF_{E}:G-CSFR(Ig)_{WT} were considered together with visual inspection to assess the strength of pairing and selectivity against mispairing with WT in order to rank designs.

Results

Designs listed in Table 4 have in silico packing metrics in ZymeCAD™, that favour pairing of G-CSF_{E}:G-CSFR(Ig)_{E}, with high selectivity over mispairing with WT G-CSF or G-CSFR(Ig).

Therefore, various site III mutations of the same variant G-CSF and receptor designs shown in Table 4 are predicted to exhibit preferential binding, over wild type G-CSFR and G-CSF, respectively.

TABLE 5
Site III designs with AMBER metrics from triplicate
in silico mean-field packs in kcal/mol in ZymeCAD^{Tm}.

| | G-CSF_{E}:G-CSFR(Ig)_{E} | | G-CSF_{E}:G-CSFR(Ig)_{WT} | G-CSF_{WT}:G-CSFR(Ig)_{E} |
|---|---|---|---|---|
| Design # | dAmber_affinity | dAmber_folding | ddAmber_affinity_E_WT | ddAmber_affinity_WT_E |
| 51 | −31.84 | 21.42 | −28.60 | −49.04 |
| 52 | −31.81 | 21.57 | −36.14 | −42.08 |
| 53 | −28.72 | 30.24 | −36.29 | −48.49 |
| 54 | −27.30 | 36.15 | −34.29 | −31.51 |
| 55 | −23.32 | 21.36 | −33.71 | −37.62 |
| 56 | −21.25 | 28.70 | −40.40 | −36.60 |
| 57 | −18.31 | 15.77 | −24.68 | −51.81 |
| 58 | −17.69 | 17.14 | −24.62 | −36.77 |
| 59 | −17.64 | 28.39 | −27.76 | −40.42 |
| 60 | −16.01 | 30.54 | −22.94 | −45.27 |
| 61 | −15.84 | 29.64 | −9.07 | −33.76 |
| 62 | −14.29 | 2.89 | −21.18 | −12.78 |
| 63 | −14.20 | 3.57 | −14.42 | −14.65 |
| 64 | −13.68 | 34.79 | −22.85 | −22.93 |
| 65 | −13.51 | 28.01 | −15.43 | −34.93 |
| 66 | −12.58 | 10.88 | −19.51 | −26.53 |
| 67 | −11.43 | 35.72 | −21.79 | −27.46 |
| 68 | −10.95 | 15.99 | −12.52 | −20.20 |
| 69 | −9.53 | 18.46 | −7.37 | −32.23 |
| 70 | −9.34 | 13.13 | −27.74 | −10.28 |
| 71 | −7.63 | 15.05 | −7.95 | −15.03 |
| 72 | −5.68 | 10.95 | −1.97 | −16.54 |

### Example 4: Combination of Site II and III to Create a Variant, Co-Evolved Cytokine-Receptor Switch

To develop a fully selective design pair G-CSF_{E}:G-CSF(Ig-CRH)_{E}, that enables binding and signaling through the 2:2 heterodimeric engineered complex and has low or completely abolished cross-reactivity with wild type cytokine or receptor, selected site II and III designs from Examples 1 and 3 were combined (see, Table 6) and tested in vitro.

In the same way the designs in Table 6 were combined, any other combination of a site II design of Example 1 (Table 2) with a site III design of Example 3 (Table 4) could result in a combined fully selective G-CSF_{E}:G-CSFR(Ig-CRH)_{E} design that enables variant signaling. Combination designs 401 and 402 were tested in the co-expression assay described in Example 2 for their ability to form an engineered G:CSF_{E}:G-CSFR(Ig-CRH)_{E} complex, as well as for their ability to bind WT cytokine or receptor.

Methods

The co-expression assay with pulldown through the cytokine TST tag was performed as described above in Example 2, with the difference that the receptor constructs contained the Ig and CRH domains (residues 2-308, Table 1) referred to as G-CSFR(Ig-CRH).

Results

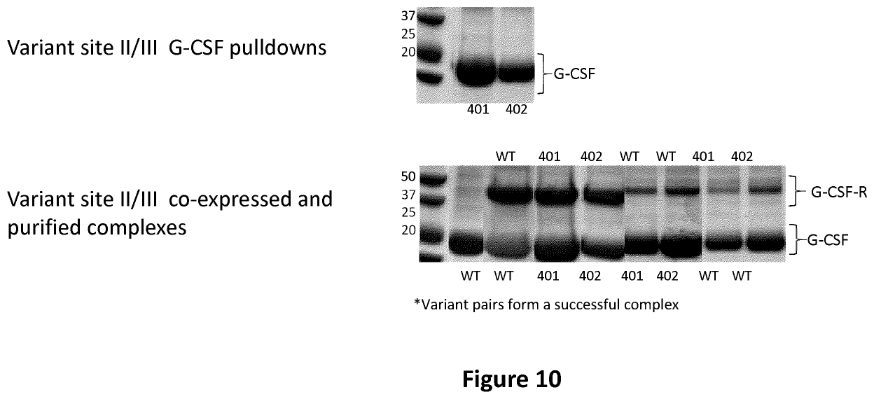

Combination designs 401 and 402 were fully selective in the co-expression assay, in that the design cytokines pulled down their co-evolved, engineered receptor but not WT receptor, and vice versa WT G-CSF did not pull down the engineered receptor (see, FIG. 10).

These results show that variant G-CSF and receptors comprising variant G-CSFR ECD designs combining select site II and site III mutations are capable of specific binding to engineered cytokine receptor pair and do not bind the wild type receptor or cytokine, respectively.

### Example 5: Production of G-CSF and G-CSFR Wildtype and Mutants

To produce wild type and engineered cytokine and receptor variants and compare their biophysical properties, recombinant proteins were expressed and purified from insect cells.

Methods

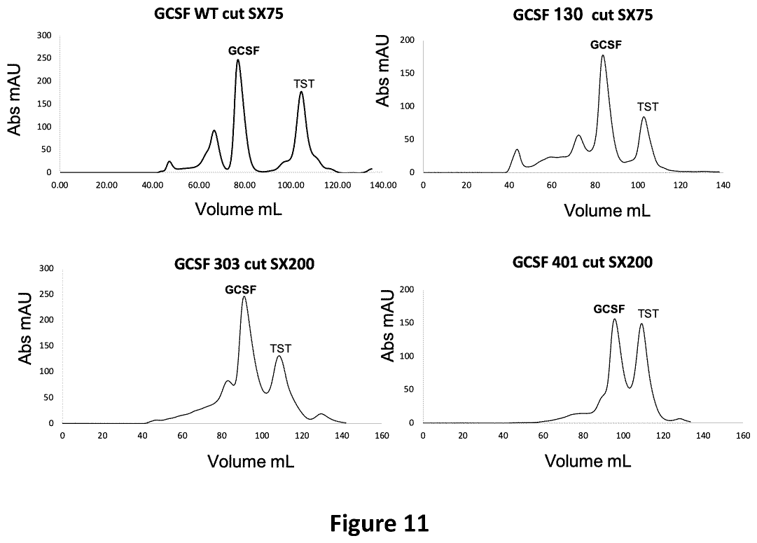

G-CSF_{E} and G-CSF_{WT} were cloned as described above. Preparative scale production of recombinant proteins was performed in 2-4 L healthy, log-phase Tni cells as follows: 800 mL Tni at 2×10⁶ cells ml⁻¹ were inoculated with 20 μl cytokine variant P2 virus per 2 ml cells, and incubated for 70 h at 27° C. with shaking at 135 rpm. After incubation, the cells were pelleted by centrifugation at 5500 rpm for 15 minutes and the supernatant filtered twice, first through a 1 μm Type A/E glass fiber filter (PALL, cat. 61631) followed by 0.45 PVDF membrane filter (Sigma Aldrich, cat. HVLP04700). Protease inhibitor cocktail III (Sigma Aldrich, cat. 539134) was added and the supernatant buffer exchanged into HBS (20 mM HEPES pH8, 150 mM NaCl) and concentrated to 300 mL on the tangential flow. Protein was purified in batch mode with 3×3 mL b.v Streptactin-XT superflow and incubated for 2×1 h, and 1× overnight at 4° C. with stirring. Prior to elution the resin was washed with 10 CV HBS buffer. Protein was eluted in 4×5 mL of BXT elution buffer. Eluates were analyzed by nanodrop A280 and reducing SDS-PAGE, concentrated to −2 mL and the TST purification tag cleaved by incubation with TEV at 1:80 ratio of TEV:protein at 18° C. overnight with end-over-end mixing. Cut protein was confirmed by SDS-PAGE prior to being loaded onto either a SX75 16/600 or SX200 16/600 size exclusion column (GE Healthcare, cat. 28-9893-33 or 28-9893-35) equilibrated in 20 mM BisTris pH 6.5, 150 mM NaCl (see, FIG. 11). Protein containing fractions were analyzed by reducing SDS-PAGE, pooled and the concentration was measured by nanodrop A280 measurement.

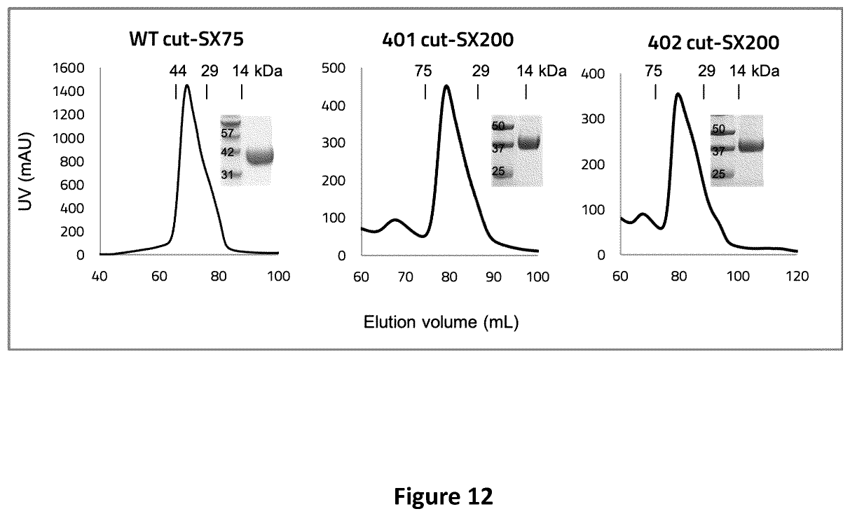

For protein purification of receptor variants, wild type and G-CSFR(Ig-CRH)_{E} mutants were cloned as described above, receptor constructs for purification included the Ig domain in addition to the CRH domain (residues 3-308 of Uniprot ID Q99062, Table 1). Viral stocks were prepared and used for infection at 2-4 L scale as described above. Clarified supernatants were buffer exchanged into Ni-NTA binding buffer (20 mM HEPES pH8, 1 M NaCl, 30 mM Imidazole) and concentrated to 300 mM as described above. Protein was purified in batch bind mode with 3×3 mL b.v Ni-NTA superflow and incubated for 2×1 h, and 1× overnight at 4° C. with stirring. Prior to elution the resin was washed with 10 CV binding buffer. Protein was eluted in 4×5 mL Ni-NTA elution buffer (20 mM HEPES pH8, 1 M NaCl, 250 mM Imidazole). Eluates were analyzed, buffer exchanged into 20 mM Bis-Tris pH 6.5, 150 mM NaCl, concentrated and cleaved overnight as described above. Cut protein was subsequently loaded onto a SX 75 16/600 or SX200 16/600 size exclusion column (GE Healthcare) equilibrated in 20 mM BisTris pH 6.5, 150 mM NaCl (see, FIG. 12). Protein containing fractions were analyzed by reducing SDS-PAGE, pooled and the concentration was measured by nanodrop A280 measurement.

Results

Wild type, G-CSF_{E} and G-CSFR_{E} mutants were >90% pure post SEC as judged by reducing SDS-PAGE (see, FIGS. 11 and 12). The yield post SEC per 1 L culture of design 401 and 402 G-CSF_{E} was 2.7 mgs and 1.6 mgs, respectively. The yield post SEC per 1 L production of design 401 and 402 G-CSFR_{E} was 1.7 mgs and 1.5 mgs, respectively. WT G-CSF was purified with a yield of 2.1 mgs post SEC per 1 L culture, WT G-CSFR was purified with a yield of 3.1 mg post SEC per 1 L of culture.

These results confirm that the methods used to purify variant G-CSF and receptors were efficient to yield purified protein to be used for analysis of biophysical properties in vitro.

### Example 6: Determination of the Affinity of Designs for their Paired and Mispaired Binding Partner by SPR

In order to determine the affinity of design cytokines for their co-evolved receptor mutant, we measured the affinity of G-CSF_{E} for G-CSFR_{E}. We also determined the affinity of G-CSF_{E} for G-CSFR_{WT} and the affinity of G-CSF_{WT} for G-CSFR_{E} for a subset of designs by SPR (mispaired).

Methods

The SPR binding assays were carried out on a Biacore T200 instrument (GE Healthcare, Mississauga, ON, Canada) with PBS-T (PBS+0.05% (v/v) Tween 20) running buffer at a temperature of 25° C. CMS Series S sensor chip, Biacore amine coupling kit (NHS, EDC and 1 M ethanolamine), and 10 mM sodium acetate buffers were all purchased from GE Healthcare. PBS running buffer with 0.05% Tween20 (PBS-T) was purchased from Teknova Inc. (Hollister, CA). Designs were assessed in three different immobilization orientations.

To determine binding affinity of G-CSF_{E} to G-CSFR_{E} the G-CSFR_{E} mutant was captured by standard amine coupling as described by the manufacturer (GE LifeSciences). Briefly, immediately after EDC/NHS activation, a 5 μg/mL solution of G-CSFR_{E} in 10 mM NaOAc, pH 5.0, was injected at a flow rate of 5 μL/min until a receptor density of ˜700-900 RU was reached. The remaining active groups were quenched by a 420 s injection of 1 M ethanolamine hydrochloride-NaOH pH 8.5 at 10 μL/min. Using single-cycle kinetics, six concentrations of a two-fold dilution series of the corresponding G-CSF_{E} mutant starting at 200 nM with a blank buffer control were sequentially injected at 25 μL/min for 300s with a 1800s dissociation phase, resulting in a set of sensorgrams with a buffer blank reference. The same sample titration was also performed on a reference cell with no variants captured. The chip was regenerated to prepare for the next injection cycle by one pulse of 10 mM glycine/HCl, pH 2.0, for 30 s at 30 μL/min.

To assess binding affinity of G-CSF_{WT} to G-CSFR_{E}, G-CSF_{E} was captured on the chip at a density of ˜700-900 RU as described above. Using single-cycle kinetics, six concentrations of a two-fold dilution series of each G-CSFR_{WT} starting at 200 nM with a blank buffer control were sequentially injected at 25 μL/min for 300s with a 1800s total dissociation time, resulting in a set of sensorgrams with a buffer blank reference. The same sample titration was also performed on a reference cell with no variants captured and the chip was regenerated as described above.

To assess binding affinity of G-CSF_{E} to G-CSFR_{WT}, recombinant G-CSFR_{WT} purified as described in Example 5 was captured as described above in this Example. Using single-cycle kinetics, six concentrations of a two-fold dilution series of each G-CSF_{E} mutant starting at 200 mM with a blank buffer control were sequentially injected as described above. The same sample titration was also performed on a reference cell with no variants captured. The G-CSFR_{WT} surface was regenerated as described above.

As a control, the binding of WT G-CSF to WT G-CSFR(Ig-CRH) was assessed in each experiment and used to calculate the K_{D} fold change within each independent measurement.

Double-referenced sensorgrams from duplicate or triplicate repeat injections were analyzed using Biacore™ T200 Evaluation Software v3.0 and fit to the 1:1 Langmuir binding model.

Results

Kinetic-derived affinity constants (K_{D}) where obtained by fitting the association and dissociation phases of the curves. The K_{D} of WT G-CSF for WT G-CSFR(Ig-CRH) ranged between 1.8-2.5E-9. For the cases in which the kinetic parameters could not be fit an attempt was made to derive a steady state affinity constant. In those cases, the KD fold change was calculated from the steady state derived K_{D} for the WT G-CSF:WT G-CSFR(Ig-CRH) pair and is indicated in Table 7.

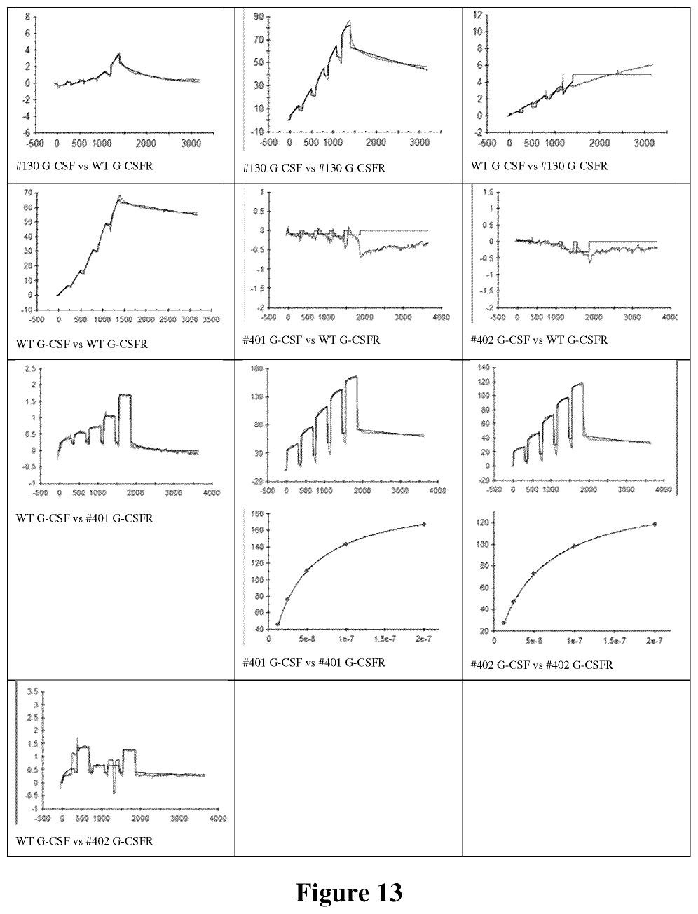

Designs 9, 130, 134, 137, 307, 401 and 402 showed affinities for their co-evolved binding partner not more than 2× different from the WT:WT KD (see Table 7 and FIG. 13).

Design 9, 30 and 34 G-CSFR(Ig-CRH)_{E} mutants showed more than >700× weaker affinity for WT G-CSF compared to WT G-CSFR(Ig-CRH). Design #35 G-CSFR(Ig-CRH)_{E} was less selective against mispairing with WT cytokine, its affinity for WT G-CSF was reduced ˜19× compared to WT:WT affinity. Designs 130, 134, 401, 402, 300, 3003, 304 and 307 G-CSFR(Ig-CRH)_{E} mutant were the most selective against mispairing with WT G-CSF and did not show appreciable binding to WT G-CSF at the concentrations titrated (see, Table 8, FIG. 13).

Design 124, 130, 401, 402, 300, 303, 304 and 307 G-CSF_{E} mutants did not show appreciable binding to WT G-CSFR(Ig-CRH) at the concentrations titrated. Design 9, 30 and 34 G-CSF_{E} mutants showed at least a ˜20× weaker K_{D} for WT G-CSFR(Ig-CRH) compared to the WT:WT KD. Design #134 G-CSF_{E} showed ˜500× weaker affinity for WT G-CSFR(Ig-CRH) (see, Table 9, FIG. 13). Design 35 and 117 G-CSF_{E} mutants show binding to the WT G-CSFR(Ig-CRH) similar to the WT:WT KD.

These results show that selected variant G-CSF designs do not bind wild type G-CSFR ECD or bind with a significantly reduced affinity to wild type G-CSFR ECD (at least ˜20× weaker KD).

TABLE 7
Change in binding affinity (KD) of G-CSF_{E} mutants
for their corresponding G-CSFR(Ig-CRH)_{E} mutant compared
to WT:WT binding affinity determined by SPR

| | K_{D} fold change |
|---|---|
| WT:WT control | — |

Design G-CSF_{E}:G-CSFR(Ig-CRH)_{E}

| | |
|---|---|
| 9 | 1.2 |
| 30 | 11 |
| 34 | 5.0 |
| 35 | 3.3 |
| 130 | 0.9 |
| 134 | 1.0 |
| 137 | 0.3 |
| 401 | 0.6^{ss} |
| 402 | 0.8^{ss} |
| 300 | 194 |
| 301 | Not measured |
| 303 | 2.2^{ss} |
| 304 | 6.5 |
| 307 | 0.6^{ss} |

^{ss}denotes a steady state derived affinity constant.

TABLE 8
Change in binding affinity of WT G-CSF to design G-CSFR(Ig-
CRH)_{E} compared to WT:WT binding affinity determined by SPR

| | K_{D} fold change |
|---|---|
| WT:WT control | — |

Design G-CSFR(Ig-CRH)_{E}

| | |
|---|---|
| 9 | 8807^{ss} |
| 30 | 774 |
| 34 | 4258^{ss} |
| 35 | 19 |
| 130 | No binding |
| 134 | No binding |
| 401 | No binding |
| 402 | No binding |
| 300 | No binding |
| 301 | Not determined |
| 303 | No binding |
| 304 | No binding |
| 307 | No binding |

^{ss}denotes a steady state derived affinity constant

TABLE 9
Change in binding affinity of G-CSF_{E} to wild type G-CSFR(Ig-
CRH) compared to WT:WT binding afffinity determined by SPR

| | K_{D} fold change |
|---|---|
| WT:WT control | — |

Design G-CSF_{E}

| | |
|---|---|
| 9 | 79 |
| 30 | 20 |
| 34 | 33 |
| 35 | 5.0 |
| 117 | 3.4 |
| 124 | No binding |
| 130 | No binding |
| 134 | 465 |
| 401 | No binding |
| 402 | No binding |
| 300 | No binding |
| 301 | Not determined |
| 303 | No binding |
| 304 | No binding |
| 307 | No binding |

### Example 7: Determination of the Thermal Stability of Design G-CSF_{E} Mutants

To determine the thermal stability of G-CSF_{E} and G-CSFR_{E} mutants in comparison to WT cytokine and receptor we performed differential scanning calorimetry (DSC).

Methods

The thermal stability of variants was assessed by Differential Scanning calorimetry (DSC) as follows: 950 mL of purified samples at concentrations of 1-2 mg/mL were used for DSC analysis with a Nano DSC (TA instruments, New Castle, DE). At the start of each run, buffer blank injections were performed for baseline stabilization. Each sample was scanned from 25 to 95° C. at a 60° C./hr rate, with 60 psi nitrogen pressure. The resulting thermograms were referenced and analyzed using NanoAnalyze software to determine melting temperature (Tm) as an indicator of thermal stability.

Results

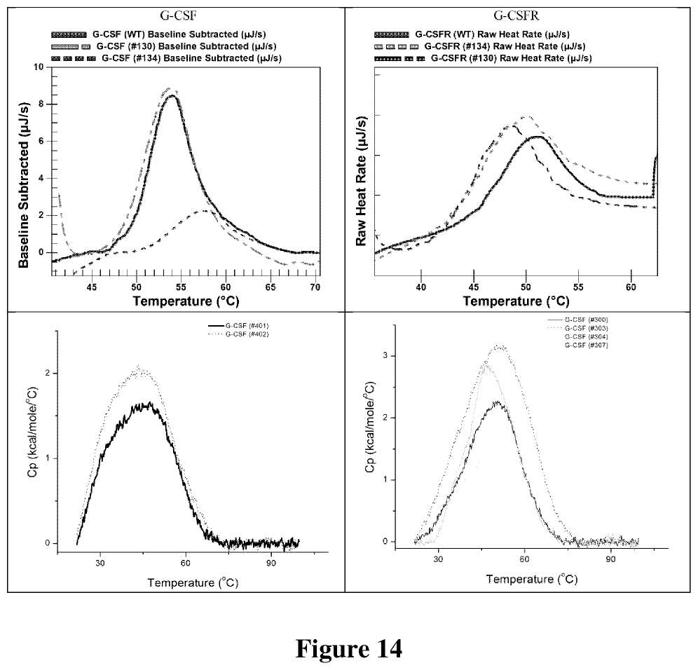

Thermal stability of the engineered variants is reported as the difference between the most prominent transition (highest enthalpy) of the engineered and the equivalent wild type molecule, measured under the same conditions and experimental set up. Measured WT GCSF Tm vary between 52.2 and 55.4° C. among independent experiments, while WT G-CSFR displays a Tm of 50.5° C. G-CSF_{E} mutants tested with the exception of designs #15 and 34 showed a Tm less than 5° C. different from the Tm for WT G-CSF (see, Table 10 and FIG. 14). All receptor mutants tested showed the same thermal stability as WT receptor (see, Table 10 and FIG. 14).

These results show that the variant G-CSF and receptors with the variant G-CSFR ECDs designs have similar thermal stability as wild type G-CSF and G-CSFR; and the site II and/or site III mutations do not disrupt thermal stability of either G-CSF or the G-CSFR ECD.

TABLE 10
Change in melting temperature (Tm) for design cytokine and
receptor mutants compared to wild type determined by DSC.

| | ΔTm (° C.) |
|---|---|
| GCSF_{E} design mutant | |
| WT | — |
| #15 | −9.9 |
| #34 | −6.1 |
| #35 | −3.5 |
| #106 | +7.5 |
| #130 | −1.8 |
| #134 | +2.0 |
| #135 | −0.8 |
| #300 | +1.0 |
| #301 | −3.2 |
| #303 | +4.2 |
| #304 | +2.7 |
| #307 | −3.8 |
| #401 | −2.5 |
| #402 | −3.8 |
| GCSFR (Ig-CRH)_{E} design mutant | |
| WT | — |
| #9 | +1.4 |
| #30 | +1.4 |
| #34 | +1.8 |
| #35 | +1.8 |
| #130* | −1.9 |
| #134 | −0.9 |
| #300 | Not measured |
| #301 | Not measured |
| #303 | Not measured |
| #304 | Not measured |
| #307 | Not measured |
| #401 | Not measured |
| #402 | Not measured |

*determined in 150 mM NaCl, 20 mM BisTris pH 6.5

### Example 8: Determination of the Monodispersity of G-CSF_{E} Mutants by UPLC-SEC

To determine the monodispersity of G-CSF_{E} mutants in comparison to WT G-CSF we analyzed mutants by UPLC-SEC.

Methods

UPLC-SEC was performed on SEC purified protein samples using an Acquity BEH125 SEC column (4.6×150 mm, stainless steel, 1.7 μm particles) (Waters LTD, Mississauga, ON) set to 30° C. and mounted on an Agilent Technologies 1260 infinity II system with a PDA detector. Run times consisted of 7 minutes with a running buffer of 150 mM NaCl, 20 mM HEPES pH 8.0 or 150 mM NaCl, 20 mM BisTris pH 6.5 at a flow rate of 0.4 mL/min. Elution was monitored by UV absorbance in the range 210-500 nm, and chromatograms were extracted at 280 nm. Peak integration was performed using OpenLAB™ CDS ChemStation™ software.

### Results

WT G-CSF and design 34, 35 and 130 G-CSF_{E} mutants were 100% monodisperse (see, Table 11). Mutants 8, 9, 15, 117, 135 showed lower monodispersity between 65.3-79.5%. Design #134 cytokine showed 57.3% monodispersity at pH 8.0 which improved to 86.6% monodispersity at pH 6.5. The improvement in monodispersity at lower pH of the mobile phase could be due to a shift of pI, for example from a calculated pI of 5.41 for WT G-CSF to a calculated pI of 8.35 for design #134 G-CSF_{E}.

These results show that some of the variant G-CSF designs have 100% monodispersity as wild type G-CSF; indicating that a sub-set of the site II and/or site III mutations do not disrupt monodispersity of G-CSF; whereas other variant G-CSF designs led to lowered monodispersity that was increased at lower pH.

TABLE 11
Monodispersity of G-CSF_{E} design
mutants determined by UPLC-SEC.

| G-CSF_{E} Design Mutant | Monodispersity % |
|---|---|
| WT | 100.0 (96.7*) |
| 8 | 73.7 |
| 9 | 65.3 |
| 15 | 57.3 |
| 34 | 100.0 |
| 35 | 100.0 |
| 117 | 74.7* |
| 130 | 100.0* |
| 134 | 57.3 (86.6*) |
| 135 | 79.5* |
| 401 | 89.6* |

*in 150 mM NaCl, BisTris pH 6.5

### Example 9: Construction of Chimeric G-CSF Receptors with Intracellular IL-2 Receptor Signalling Domains

To investigate whether design G-CSF_{E} cytokine mutants are able to signal and cause immune cell proliferation through the design G-CSFR(Ig-CRH)_{E} receptor mutants, we constructed a single-chain chimeric G-CSF receptor using the G-CSFR ECD fused to the gp130 transmembrane (TM) domain and intracellular signaling domain (ICD), and an IL-2Rβ intracellular signaling domain (G-CSFR_{WT}-ICD_{gp130-IL-2Rβ}). We also utilized a chimeric G-CSFR that consists of two subunits designed to be co-expressed as a heterodimeric receptor: 1) The G-CSFR_{WT}-ICD_{IL-2Rβ} subunit consists of the G-CSFR ECD fused to the IL-2Rβ TM and ICD; and 2) The G-CSFR_{WT}-ICD_{γc} subunit consists of the G-CSFR ECD fused to the common gamma chain (γc, IL-2Rγ2Rγ) TM and ICD.

Methods

The single-chain chimeric receptor construct was designed to include the G-CSFR signal peptide and ECD, followed by the gp130 TM and partial ICD, and IL-2Rβ partial ICD (Table 12). The heterodimeric chimeric receptor construct was designed to include: 1) the G-CSFR signal peptide and ECD, followed by the IL-2Rβ TM and ICD (Table 13); and 2) the G-CSFR signal peptide and ECD, followed by the γc TM and ICD (Table 14). The chimeric receptor constructs were cloned into a lentiviral transfer plasmid and the construct sequences were verified by Sanger sequencing. The transfer plasmid and lentiviral packaging plasmids (psPAX2, pVSVG) were co-transfected into lentiviral packaging cell line HEK293T/17 cells (ATCC) as follows: Cells were plated overnight in DMEM containing 10% fetal bovine serum and penicillin/streptomycin, and medium changed 2-4 hours prior to transfection. Plasmid DNA and water were mixed in a polypropylene tube, and CaCl₂) (0.25M) was added dropwise. After a 2 to 5 minute incubation, the DNA was precipitated by mixing 1:1 with 2× HEPES-buffered saline (0.28M NaCl, 1.5 mM Na₂HPO₄, 0.1M HEPES). The precipitated DNA mixture was added onto the cells, which were incubated overnight at 37° C., 5% CO2. The following day the HEK293T/17 medium was changed, and the cells were incubated for another 24 hours. The following morning, the cellular supernatant was collected from the plates, centrifuged briefly to remove debris, and filtered through a 0.45 μm filter. The supernatant was spun for 90 minutes at 25,000 rpm using a SW-32Ti rotor in a Beckman Optima L-XP Ultracentrifuge. The supernatant was removed, and the pellet was resuspended in a suitable volume of Opti-MEM medium. The viral titer was determined by adding serial dilutions of virus onto BAF3 cells (grown RPMI containing 10% fetal bovine serum, penicillin, streptomycin and 100 IU/ml hIL-2). 48-72 hours after transduction, the cells were incubated with an anti-human G-CSFR APC-conjugated antibody (1:50 dilution) and eBioscience™ Fixable Viability Dye eFluor™ 450 (1:1000 dilution) for 15 minutes at 4° C., washed, and analyzed on a Cytek Aurora or BD FACS Calibur flow cytometer. Using the estimated titer determined by this method, the 32D-IL-2Rβ cell line (grown in RPMI containing 10% fetal bovine serum, penicillin, streptomycin and 300 IU/ml hIL-2) was transduced with the lentiviral supernatant encoding the chimeric receptor construct at an MOI of 0.5. Transduction was performed by adding the relevant amount of viral supernatant to the cells, incubating for 24 hours, and replacing the cell medium. 3-4 days after transduction, we verified expression of the human G-CSFR by flow cytometry, as described above. Cells were expanded in G-CSF_{WT} for approximately 14-28 days before performing the BrdU assay.

32D-IL-2Rβ cells expanded in G-CSF_{WT} as described above were washed three times in PBS, and re-plated in fresh medium containing the relevant assay cytokine (no cytokine, hIL-2 (300 IU/ml), G-CSF_{WT} (30 ng/ml) or G-CSF_{E} (30 ng/ml) for 48 hours. The BrdU assay procedure followed the instruction manual for the BD Pharmingen™ APC BrdU Flow Kit (557892), with the following addition: cells were co-incubated with BrdU and eBioscience™ Fixable Viability Dye eFluor™ 450 (1:5000) for 30 minutes. Analytical flow cytometry was performed using a Cytek Aurora instrument.

Results

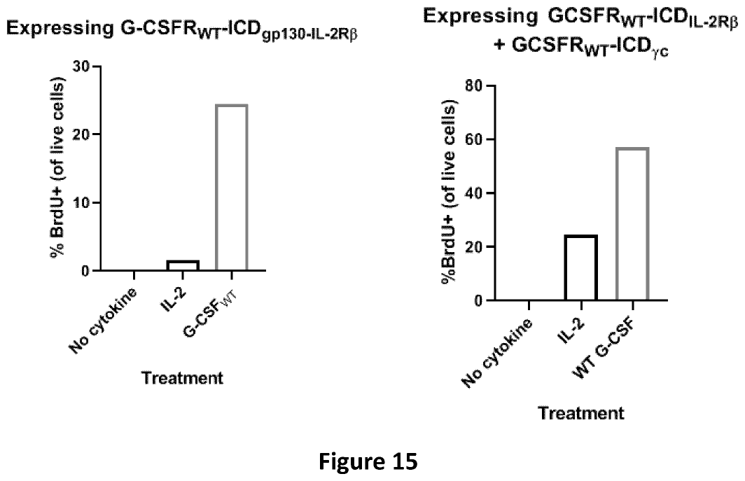

In a BrdU assay, cells transfected with the single-chain chimeric receptor construct G-CSFR_{WT}-ICD_{gp130-IL-2Rβ} or the heterodimeric receptor construct G-CSFR_{WT}-ICD_{IL-2Rβ} plus G-CSFR_{WT}-ICD_{γc} exhibited similar or superior proliferation in response to G-CSF_{WT} (30 ng/ml) compared to hIL-2 (300 IU/ml). Cells did not proliferate in the absence of cytokine (see, FIG. 15).

These results show that, upon stimulation by G-CSF, single chain and heterodimeric chimeric receptor constructs can be activated to induce cell proliferation.

### Example 10: Proliferation of 32D-IL-2Rβ Cells Transduced with Design 137 G-CSFR_{E}-ICD_{IL-2} and Treated with Wild Type or Design G-CSF₁₃₇ Examined by BrdU

Site II/III combination designs that were sufficiently selective as judged by SPR or co-expression assays were tested in vitro for the ability to induce proliferation of 32D-IL-2Rβ cells.

Methods

Point mutations for design 137 were introduced into the constructs described in Tables 12-14. Cloning and expression of the G-CSFR₁₃₇-ICD_{gp130-IL-2Rβ} (homodimer) or G-CSFR₁₃₇-ICD_{IL-2Rβ} plus G-CSFR₁₃₇-ICD_{γc} (heterodimer) constructs followed the same procedures as described above. Before performing the BrdU assay, cells were expanded in G-CSF 137 for approximately 14-28 days.

32D-IL-2Rβ cells expanded in G-CSF₁₃₇ were assayed for proliferation in G-CSF₁₃₇ (30 ng/ml), G-CSF_{WT} (30 ng/ml), hIL-2 (300 IU/ml) or no cytokine using the BrdU assay procedure described above.

Results

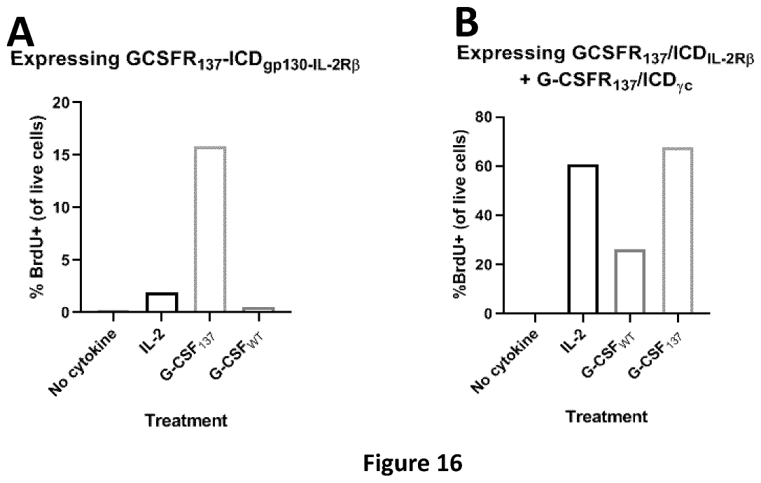

In a BrdU assay, cells transduced with the single-chain chimeric receptor construct G-CSFR₁₃₇-ICD_{gp130-IL-2Rβ} or the heterodimeric receptor construct G-CSFR₁₃₇-ICD_{IL-2Rβ} plus G-CSFR₁₃₇-ICD_{γc} exhibited similar or superior proliferation in G-CSF₁₃₇ (30 ng/ml) compared to hIL-2 (300 IU/ml). Cells did not proliferate in absence of cytokine and exhibited inferior proliferation in G-CSF_{WT} (30 ng/ml) (see, FIG. 16).

These results indicate that the variant G-CSF specifically activates the engineered receptor; and conversely, the engineered receptor is activated by the variant G-CSF but markedly less so than wild type G-CSF. Therefore, variant G-CSF can specifically activate chimeric receptors with variant G-CSFR ECD to specifically induce proliferation of cells expressing the chimeric receptors.

### Example 11: Proliferation of 32D-IL-2Rβ Cells Transduced with Wild-Type G-CSFR-ICD_{IL-2} and Treated with Wild Type or Design G-CSF_{E} Examined by BrdU

Site II/III combination designs that were able to restore paired signaling in 32D-IL-2R2Rβ cells were subsequently tested for their ability to induce proliferation of 32D-IL-2R2Rβ cells transduced with the single-chain chimeric receptor construct G-CSFR_{WT}-ICD_{gp130-IL-2Rβ} or the heterodimeric receptor construct G-CSFR_{WT}-ICD_{IL-2Rβ} plus G-CSFR_{WT}-ICD_{γc}.

Methods

Cloning and expression of the G-CSFR_{WT}-ICD_{gp130-IL-2Rβ} (homodimer) or G-CSFR_{WT}-ICD_{IL-2Rβ} plus G-CSFR_{WT}-ICD_{γc} (heterodimer) constructs followed the same procedures as described above. Before performing the BrdU assay, cells were expanded in G-CSF_{WT} for approximately 14-28 days.

32D-IL-2Rβ cells expanded in G-CSF_{WT} were assayed for proliferation in G-CSF₁₃₇ (30 ng/ml), G-CSF_{WT} (30 ng/ml), hIL-2 (300 IU/ml) or no cytokine using the BrdU assay procedure described above.

Results

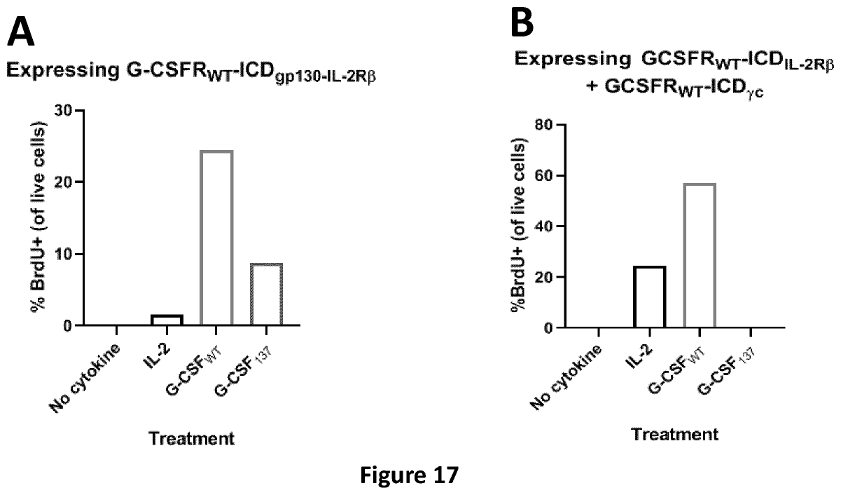

In a BrdU assay, cells transduced with the single-chain chimeric receptor construct G-CSFR_{WT}-ICD_{130-IL-2Rβ} or the heterodimeric receptor construct G-CSFR_{WT}-ICD_{IL-2Rβ} and G-CSFR_{WT}-ICD_{γc} exhibited inferior proliferation in G-CSF₁₃₇ (30 ng/ml) compared to G-CSF_{WT} (30 ng/ml). Cells did not proliferate in the absence of cytokine (see, FIG. 17).

These results indicate that the variant G-CSF does not efficiently bind wild type G-CSFR, and the variant G-CSF specifically activates the engineered receptor, but not wild type G-CSFR, to induce cell proliferation.

### Example 12: Signaling in 32D-IL-2Rβ Cells Transduced with WT or Design 137 G-CSFR_{E}-ICD_{IL-2} and Treated with Wild Type or Design G-CSF₁₃₇ Analyzed by Western Blot

Site II/III combination designs, that were able to restore proliferative signaling through the engineered cytokine-receptor complex in 32D-IL-2Rβ cells and did not significantly signal through binding G-CSF_{WT} or WT-GCSFR-ICD-IL2, were assessed by western blot for the ability to activate downstream signaling molecules in response to G-CSF_{WT} or G-CSF₁₃₇.

Methods

Cloning and expression of the G-CSFR_{WT}-ICD_{gp130-IL-2Rβ} (homodimer) or G-CSFR_{WT}-ICD_{IL-2Rβ} plus G-CSFR_{WT}-ICD_{γc} (heterodimer) constructs followed the same procedures as described above. Cells were expanded in G-CSF₁₃₇ or G-CSF_{WT} for approximately 14-28 days before performing the western blot assay. Non-transduced cells were maintained in IL-2.

To perform western blots, cells were washed three times in PBS and rested in medium containing no cytokine for 16-20 hours. Cells were stimulated with no cytokine, IL-2 (300 IU/ml), G-CSF₁₃₇ (30 ng/ml) or G-CSF_{WT} (30 ng/ml) for 20 minutes at 37° C. Cells were washed once in a wash buffer containing 10 mM HEPES, pH 777.9, 1 mM MgCl2, 0.05 mM EGTA, 0.5 mM EDTA, pH 8.0, 1 mM DTT, and 1× Pierce Protease and Phosphatase Inhibitor Mini Tablets (A32961). Cells were lysed in the wash buffer above, with the addition of 0.2% Igepal CA630 (Sigma), for 10 minutes on ice and centrifuged for 10 minutes at 13,000 rpm at 4° C., after which supernatant (cytoplasmic fraction) was collected. The pellet was resuspended and lysed in the above wash buffer with the addition of 0.42M NaCl and 20% glycerol. Cells were lysed for 30 minutes on ice, with frequent vortexing, and centrifuged for 20 minutes at 13,000 rpm at 4° C., after which the nuclear fraction (supernatant) was collected. The cytoplasmic and nuclear fractions were reduced (70° C.) for 10 minutes and run on a NuPAGE™ 4-12% Bis-Tris Protein Gel. The gels were transferred to nitrocellulose membrane (60 min at 20V in a Trans-Blot® SD Semi-Dry Transfer Cell), dried, and blocked for 1 hr in Odyssey® Blocking Buffer in TBS (927-50000). The blots were incubated with primary antibodies (1:1,000) overnight at 4° C. in Odyssey® Blocking Buffer in TBS containing 0.1% Tween20. The primary antibodies utilized were obtained from Cell Signaling Technologies: Phospho-Shc (Tyr239/240) Antibody #2434, Phospho-Akt (Ser473) (D9E) XP® Rabbit mAb #4060, Phospho-S6 Ribosomal Protein (Ser235/236) Antibody #2211, Phospho-p44/42 MAPK (Erk1/2) (Thr202/Tyr204) Antibody #9101, β-Actin (13E5) Rabbit mAb #4970, Phospho-Stat3 (Tyr705) (D3A7) XP® Rabbit mAb #9145, Phospho-Stat5 (Tyr694) (C1105) Rabbit mAb #9359, and Histone H3 (96C10) Mouse mAb #3638. Blots were washed three times in TBS containing 0.1% Tween20 and incubated with secondary antibodies (1:10,000) in TBS buffer containing 0.1% Tween20 for 30-60 minutes at room temperature. The secondary antibodies obtained from Cell Signaling Technologies: Anti-mouse IgG (H+L) (DyLight™ 800 4×PEG Conjugate) #5257 and Anti-rabbit IgG (H+L) (DyLight™ 800 4×PEG Conjugate) #5151. Blots were washed and exposed on a LI-COR Odyssey imager.

Results

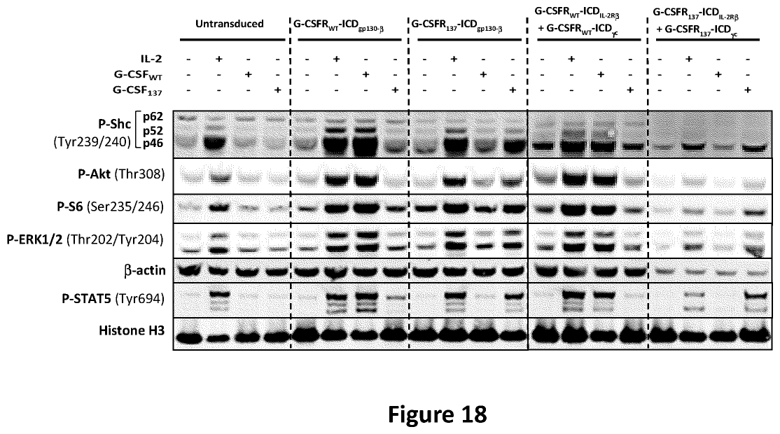

In non-transduced 32D-IL-2Rβ cells, we detected activated IL-2R-associated signaling molecules in response to stimulation with IL-2 only. We observed similar patterns of activated signaling molecules in response to IL-2 or G-CSF_{WT} in 32D-IL-2Rβ cells expressing either G-CSFR_{WT}-ICD_{gp130-IL-2Rβ} or G-CSFR_{WT}-ICD_{IL-2Rβ} plus G-CSFR_{WT}-ICD_{γc}. We did not observe activation of IL-2R-associated signaling molecules in response to G-CSF₁₃₇ stimulation of 32D-IL-2Rβ cells expressing G-CSFR_{WT}-ICD_{gp130-IL-2Rβ} or cells expressing G-CSFR_{WT}-ICD_{IL-2Rβ} plus G-CSFR_{WT}-ICD_{γc}. We observed similar patterns of activated signaling molecules in response to IL-2 or G-CSF₁₃₇ in 32D-IL-2Rβ cells expressing G-CSFR₁₃₇-ICD_{gp130-IL-2Rβ} or G-CSFR₁₃₇-ICD_{IL-2Rβ} plus G-CSFR₁₃₇-ICD_{γc}. We did not observe activation of IL-2R2R2R-associated signaling molecules in response to G-CSF_{WT} stimulation in 32D-IL-2Rβ cells expressing G-CSFR₁₃₇-ICD_{gp130-IL-2Rβ} or cells expressing G-CSFR₁₃₇-ICD_{IL-2Rβ} plus G-CSFR₁₃₇-ICD_{γc} (see FIG. 18).

These results demonstrate that variant G-CSF is capable of activating chimeric receptors expressing variant G-CSFR ECD to induce aspects of native cytokine signaling in cells expressing the chimeric receptors.

Methods for Examples 13-30

Primary cells and cell lines: The lentiviral packaging cell line HEK293T/17 (ATCC) was cultured in DMEM containing 10% fetal bovine serum and penicillin/streptomycin. BAF3-IL-2Rβ cells were previously generated by stable transfection of the human IL-2Rβ subunit into the BAF3 cell line, and were grown in RPMI-1640 containing 10% fetal bovine serum, penicillin, streptomycin and 100 IU/ml human IL-2 (hIL-2) (PROLEUKIN®, Novartis Pharmaceuticals Canada). The 32D-IL-2Rβ cell line was previously generated by stable transfection of the human IL-2Rβ subunit into the 32D cell line, and was grown in RPMI-1640 containing 10% fetal bovine serum, penicillin, streptomycin and 300 IU/ml hIL-2, or other cytokines, as indicated. Human PBMC-derived T cells (Hemacare) were grown in TexMACS™ Medium (Milenyi Biotec, 130-097-196), containing 3% Human AB Serum (Sigma-Aldrich, H4522), and 300 IU/ml hIL-2, or other cytokines, as indicated. Human tumor-associated lymphocytes (TAL) were generated by culture of primary ascites samples for 14 days in T cell medium (a 50:50 mixture of the following: 1) RPMI-1640 containing 10% fetal bovine serum, 50 uM β-mercaptoethanol, 10 mM HEPES, 2 mM L-glutamine, penicillin, streptomycin; and 2) AIM V™ Medium (ThermoFisher, 12055083) containing a final concentration of 3000 IU/ml hIL-2. Following this high-dose IL-2 expansion, TAL were cultured in T cell medium containing 300 IU/ml hIL-2 or other cytokines, as indicated. The retroviral packaging cell line Platinum-E (Cell Biolabs, RV-101) was cultured in DMEM containing 10% FBS, penicillin/streptomycin, puromycin (1 mcg/ml), and blasticidin (10 mcg/ml).

Lentiviral production and transduction of 32D-IL-2Rβ cells: Chimeric receptor constructs were cloned into a lentiviral transfer plasmid and the resulting sequences were verified by Sanger sequencing. The transfer plasmid and lentiviral packaging plasmids were co-transfected into HEK293T/17 cells using the calcium phosphate transfection method as follows: Cells were plated overnight, and medium changed 2-4 hours prior to transfection. Plasmid DNA and water were mixed in a polypropylene tube, and CaCl₂ (0.25 M) was added dropwise. After a 2 to 5 minute incubation, the DNA was precipitated by mixing 1:1 with 2× HEPES-buffered saline (0.28M NaCl, 1.5 mM Na₂HPO₄, 0.1M HEPES). The precipitated DNA mixture was added onto the cells, which were incubated overnight at 37° C., 5% CO2. The following day the HEK293T/17 medium was changed, and the cells were incubated for another 24 hours. The following morning, the cellular supernatant was collected from the plates, centrifuged briefly to remove debris, and the supernatant was filtered through a 0.45 micron filter. The supernatant was spun for 90 minutes at 25000 rpm with a SW-32Ti rotor in a Beckman Optima L-XP Ultracentrifuge. The supernatant was removed and the pellet was resuspended in a suitable volume of Opti-MEM medium. The viral titer was determined by adding serial dilutions of virus onto BAF3-IL-2Rβ cells. At 48-72 hours after transduction, the cells were incubated with an anti-human G-CSFR APC-conjugated antibody (1:50; Miltenyi Biotec, 130-097-308) and Fixable Viability Dye eFluor™ 450 (1:1000, eBioscience™, 65-0863-14) for 15 minutes at 4° C., washed, and analyzed on a Cytek Aurora or BD FACS Calibur flow cytometer. Using the estimated titer determined by this method, the 32D-IL-2Rβ cell line was transduced with the lentiviral supernatant encoding the chimeric receptor construct at a Multiplicity of Infection (MOI) of 0.5. Transduction was performed by adding the relevant amount of viral supernatant to the cells, incubating for 24 hours, and then replacing the medium. At 3-4 days after transduction, the expression of human G-CSFR was determined by flow cytometry, as described above.

Lentiviral transduction of human primary T cells: For transduction of PBMC-derived T cells and TAL, cells were thawed and plated in the presence of Human T Cell TransAct™ (Miltenyi Biotec, 130-111-160), according to the manufacturer's guidelines. 24 hours after activation, lentiviral supernatant was added, at MOI of 0.125-0.5. 48 hours after activation, the cells split into fresh medium, to remove residual virus and activation reagent. Two to four days after transduction, the transduction efficiency was determined by flow cytometry, as described above. For experiments in which the transduction efficiency of CD4+ and CD8+ fractions was determined separately, antibodies against human G-CSFR, CD4 (1:50, Alexa Fluor® 700 conjugate, BioLegend, 300526), CD8 (1:50, PerCP conjugate, BioLegend, 301030), CD3 (1:50, Brilliant Violet 510™ conjugate, BioLegend, 300448) and CD56 (1:50, Brilliant Violet 711™ conjugate, BioLegend, 318336) were utilized, along with Fixable Viability Dye eFluor™ 450 (1:1000).

Human T cell and 32D-IL-2Rβ expansion assays: Human primary T cells or 32-IL-2Rβ cells expressing the indicated chimeric receptor constructs, generated above, were washed three times in PBS and re-plated in fresh medium, or had their medium gradually changed, as indicated. Complete medium was changed to contain either wild-type human G-CSF (generated in-house or NEUPOGEN®, Amgen Canada), mutant G-CSF (generated in-house), hIL-2, or no cytokine. Every 3-5 days, cell viability and density were determined by Trypan Blue exclusion, and fold expansion was calculated relative to the starting cell number. G-CSFR expression was assessed by flow cytometry as described above.

CD4+ and CD8+ human TAL expansion assay: To examine the expansion of the CD4+ and CD8+ fractions of TAL, ex vivo ascites samples were thawed, and the CD4+ and CD8+ fractions were enriched using the Human CD4+ T Cell Isolation Kit (Miltenyi Biotec, 130-096-533) and Human CD8+ T Cell Isolation Kit (Miltenyi Biotec, 130-096-495), respectively. After expansion in cytokine-containing medium, the immunophenotype of the cells was assessed by flow cytometry, utilizing antibodies against human G-CSFR, CD4 (1:50, Alexa Fluor® 700 conjugate, BioLegend, 300526), CD8 (1:50, PerCP conjugate, BioLegend, 301030), CD3 (1:50, Brilliant Violet 510™ conjugate, BioLegend, 300448) and CD56 (1:50, Brilliant Violet 711™ conjugate, BioLegend, 318336), along with Fixable Viability Dye eFluor™ 450 (1:1000).

Primary human T cell immunophenotyping assay: After expansion in cytokine-containing medium, the immunophenotype of T cells was assessed by flow cytometry, utilizing antibodies against human G-CSFR, CD4 (1:100, Alexa Fluor® 700 conjugate, BioLegend, 300526 or PE conjugate, eBioscience™, 12-0048-42, or Brilliant Violet 570™ conjugate, Biolegend, 317445), CD8 (1:100, PerCP conjugate, BioLegend, 301030), CD3 (1:100, Brilliant Violet 510™ or Brilliant Violet 750™ conjugate, BioLegend, 300448 or 344845), CD56 (1:100, Brilliant Violet 711™ conjugate, BioLegend, 318336), CCR7 (1:50, APC/Fire™ 750 conjugate, Biolegend, 353246), CD62L (1:33, PE/Dazzle™ 594 conjugate, Biolegend, 304842), CD45RA (1:33, FITC conjugate, Biolegend, 304148), CD45RO (1:25, PerCP-eFluor® 710 conjugate, eBioscience™, 46-0457-42), CD95 (1:33, PE-Cyanine7 conjugate, eBioscience™, 25-0959-42), along with Fixable Viability Dye eFluor™ 450 or 5106 (1:1000).

Retroviral transduction: The pMIG transfer plasmid (plasmid #9044, Addgene) was altered by restriction endonuclease cloning to remove the IRES-GFP (BglII to PacI sites), and introduce annealed primers encoding a custom multiple cloning site. The chimeric receptor constructs were cloned into the customized transfer plasmid and the resulting sequences were verified by Sanger sequencing. The transfer plasmid was transfected into Platinum-E cells using the calcium phosphate transfection method, as described above. 24 hours after transfection the medium was changed to 5 ml fresh complete medium. 48 hours after transfection the cellular supernatant was collected from the plates and filtered through a 0.45 micron filter. Hexadimethrine bromide (1.6 mcg/ml, Sigma-Aldrich) and murine IL-2 (2 ng/ml, Peprotech) were added to the supernatant. This purified retroviral supernatant was used to transduce murine lymphocytes as described below.

48 hours prior to collection of retroviral supernatant, 24-well adherent plates were coated with unconjugated anti-murine CD3 (5 mcg/ml, BD Biosciences, 553058) and anti-murine CD28 (1 mcg/ml, BD Biosciences, 553294) antibodies, diluted in PBS, and stored at 4 degrees Celsius. 24 hours prior to collection of retroviral supernatant, C57Bl/6 J mice (generated in-house) were euthanized under an approved Animal Use Protocol administered by the University of Victoria Animal Care Committee. Spleens were harvested and murine T cells were isolated as follows: Spleens were manually dissociated and filtered through a 100 micron filter. Red blood cells were lysed by incubation in ACK lysis buffer (Gibco, A1049201) for five minutes at room temperature, followed by one wash in serum-containing medium. CD8a-positive or Pan-T cells were isolated using specific bead-based isolation kits (Miltenyi Biotec, 130-104-075 or 130-095-130, respectively). Cells were added to plates coated with anti-CD3- and anti-CD28-antibodies in murine T cell expansion medium (RPMI-1640 containing 10% FBS, penicillin/streptomycin, 0.05 mM β-mercaptoethanol, and 2 ng/ml murine IL-2 (Peprotech, 212-12)) or 300 IU/mL human IL-2 (Proleukin), and incubated at 37 degrees Celsius, 5% CO2 for 24 hours. On the day of transduction, approximately half of the medium was replaced with retroviral supernatant, generated above. Cells were spinfected with the retroviral supernatant at 1000 g, for 90 minutes, at 30 degrees Celsius. The plates were returned to the incubator for 0-4 hours, and then approximately half of the medium was replaced with fresh T cell expansion medium. The retroviral transduction was repeated 24 hours later, as described above, for a total of two transductions. 24 hours after the final transduction, the T cells were split into 6-well plates and removed from antibody stimulation.

48-72 hours after transduction, the transduction efficiency was assessed by flow cytometry to detect human G-CSFR, CD4 (Alexa Fluor 532 conjugate, eBioscience™, 58-0042-82), CD8a (PerCP-eFluor 710 conjugate, eBioscience™, 46-0081-82) and Fixable Viability Dye eFluor™ 450 (1:1000 dilution), as described above.

BrdU incorporation assay: Human primary T cells, 32D-IL-2Rβ cells, or murine primary T cells, generated as described above, were washed three times in PBS, and re-plated in fresh medium containing the relevant assay cytokine: no cytokine, hIL-2 (3001U/ml), wildtype or engineered G-CSF (at concentrations indicated in individual experiments) for 48 hours. The BrdU assay procedure followed the instruction manual for the BD Pharmingen™ APC BrdU Flow Kit (BD Biosciences, 557892), with the following additions: Cells were co-incubated with BrdU and Fixable Viability Dye eFluor™ 450 (1:5000) for 30 minutes to 4 hours at 37 degrees Celsius. Flow cytometry was performed using a Cytek Aurora instrument. To specifically assess the proliferation of murine T cells expressing chimeric receptors, additional staining for human G-CSFR (1:20 dilution), CD4 (1:50 dilution) and CD8 (1:50 dilution) was performed for 15 minutes on ice, prior to fixation.

Western blots: Human primary T cells, 32D-IL-2Rβ cells, or murine primary T cells, generated as described above, were washed three times in PBS and rested in medium containing no cytokine for 16-20 hours. Cells were stimulated with no cytokine, IL-2 (300 IU/ml), wildtype G-CSF (at concentrations indicated in individual experiments), or G-CSF₁₃₇ (30 ng/ml) for 20 minutes at 37 degrees Celsius. Cells were washed once in a buffer containing 10 mM HEPES, pH7.9, 1 mM MgCl₂, 0.05 mM EGTA, 0.5 mM EDTA, pH 8.0, 1 mM DTT, and 1× Pierce Protease and Phosphatase Inhibitor Mini Tablets (A32961). Cells were lysed in the wash buffer above, with the addition of 0.2% NP-40 (Sigma) for 10 minutes on ice. The lysate was centrifuged for 10 minutes at 13000 rpm at 4 degrees Celsius, and the supernatant (cytoplasmic fraction) was collected. The pellet (containing nuclear proteins) was resuspended in the wash buffer above, with the addition of 0.42M NaCl and 20% glycerol. Nuclei were incubated for 30 minutes on ice, with frequent vortexing, and the supernatant (nuclear fraction) was collected after centrifuging for 20 minutes at 13,000 rpm at 4 degrees Celsius. The cytoplasmic and nuclear fractions were reduced (70 degrees Celsius) for 10 minutes and run on a NuPAGE™ 4-12% Bis-Tris Protein Gel. The gels were transferred to nitrocellulose membrane (60 min at 20V in a Trans-Blot® SD Semi-Dry Transfer Cell), dried, and blocked for 1 hr in Odyssey® Blocking Buffer in TBS (927-50000). The blots were incubated with primary antibodies (1:1000) overnight at 4 degrees Celsius in Odyssey® Blocking Buffer in TBS containing 0.1% Tween20. The primary antibodies utilized were obtained from Cell Signaling Technologies: Phospho-Jak1 (Tyr1034/1035) (D7N4Z) Rabbit mAb #74129, Phospho-Jak2 (Tyr1007/1008) #3771, Phospho-Jak3 (Tyr980/981) (D44E3) Rabbit mAb #5031, Phospho-p70 S6 Kinase (Thr421/Ser424) Antibody #9204, Phospho-Shc (Tyr239/240) Antibody #2434, Phospho-Akt (Ser473) (D9E) XP® Rabbit mAb #4060, Phospho-S6 Ribosomal Protein (Ser235/236) Antibody #2211, Phospho-p44/42 MAPK (Erk1/2) (Thr202/Tyr204) Antibody #9101, β-Actin (13E5) Rabbit mAb #4970, Phospho-STAT1 (Tyr701) (58D6) Rabbit mAb #9167, Phospho-STAT3 (Tyr705) (D3A7) XP® Rabbit mAb #9145, Phospho-STAT4 (Tyr693) Antibody #5267, Phospho-STAT5 (Tyr694) (C1105) Rabbit mAb #9359, and Histone H3 (96C10) Mouse mAb #3638. Blots were washed three times in TBS containing 0.1% Tween20 and incubated with secondary antibodies (1:10,000) in TBS buffer containing 0.1% Tween20 for 30-60 minutes at room temperature. The secondary antibodies obtained from Cell Signaling Technologies were Anti-mouse IgG (H+L) (DyLight™ 800 4×PEG Conjugate) #5257 and Anti-rabbit IgG (H+L) (DyLight™ 800 4×PEG Conjugate) #5151. Blots were washed and exposed on a LI-COR Odyssey imager.

Flow cytometry to detect phosphorylated proteins: Human primary T cells, 32D-IL-2Rβ cells, or murine primary T cells, generated as described above, were washed three times in PBS and rested in medium containing no cytokine for 16-20 hours. Cells were stimulated with no cytokine, IL-2 (300 IU/ml) or wild-type G-CSF (100 ng/ml) for 20 minutes at 37 degrees Celsius, in the presence of Fixable Viability Dye eFluor™ 450 (1:1000), and anti-G-CSFR (1:20), anti-CD4 (1:50) and anti-CD8a (1:50), as indicated. Cells were pelleted and fixed with BD Phosflow™ Fix Buffer I (BD Biosciences, 557870) for 15 minutes at room temperature. Cells were washed, then permeabilized using BD Phosflow™ Perm Buffer III (BD Biosciences, 558050) on ice for 15 minutes. Cells were washed twice and resuspended in buffer containing 20 ul of BD Phosflow™ PE Mouse Anti-Stat3 (pY705) (BD Biosciences, 612569) or PE Mouse IgG2a, κ Isotype Control (BD Biosciences, 558595). Cells were washed and flow cytometry was performed using a Cytek Aurora instrument.

### Example 13: Expansion of Human T Cells Expressing G2R-1, G-CSFR/IL-2Rb Subunit Alone, the Myc-Tagged G-CSFR/Gamma-C Subunit Alone, or the Full-Length G-CSFR

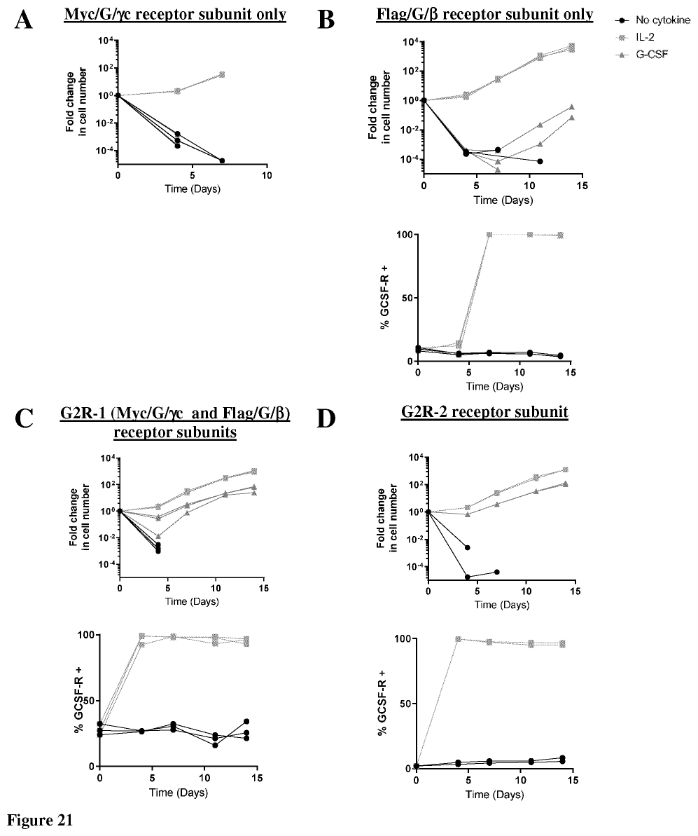

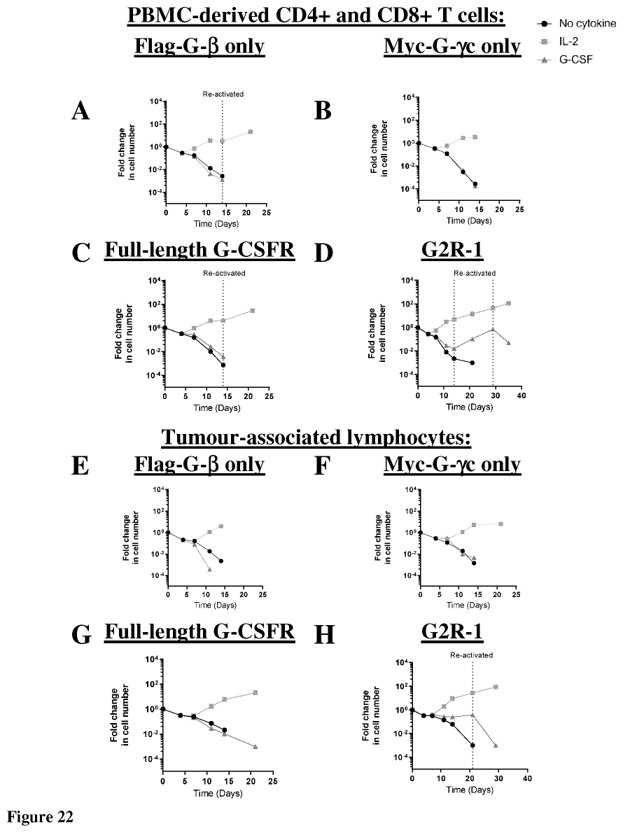

PBMC-derived T cells or tumor-associated lymphocytes (TAL) were transduced with lentiviruses encoding the chimeric receptor constructs shown in FIG. 1, and cells were washed and re-plated in the indicated cytokine. Cells were counted every 3-4 days. G/γc was tagged at its N-terminus with a Myc epitope (Myc/G/γc), and G/IL-2Rβ was tagged at its N-terminus with a Flag epitope (Flag/G/IL-2Rβ); these epitope tags aid detection by flow cytometry and do not impact the function of the receptors. As expected, all T cell cultures showed proliferation in response to the positive control cytokine, IL-2 (300 IU/ml). After stimulation with G-CSF (100 ng/ml), proliferation was observed only for PMBC-derived T cells and TAL expressing the G2R-1 chimeric cytokine receptor (FIG. 22). Note that the lentiviral transduction efficiency was less than 100% such that less than 100% of T cells expressed the indicated chimeric cytokine receptors, which likely accounts for the lower rate of proliferation mediated by G2R-1 relative to IL-2. Similarly, increased proliferation was observed in 32D-IL-2Rβ cells (stably expressing the human IL-2Rβ subunit) expressing G-CSFR chimeric receptor subunits G2R-1 and G2R-2 and stimulated with G-CSF (FIG. 21). In contrast to T cells, 32D-IL-2Rβ cells expressing the G/IL-2Rβ chimeric receptor subunit alone proliferated in response G-CSF (FIG. 2); G-CSF-induced proliferation was not seen in 32D-IL-2Rβ cells expressing the G/γc chimeric receptor subunit alone (FIG. 2).

These results show that G-CSF is capable of stimulating proliferation and viability of PMBC-derived T cells and TALs expressing the G2R-1 chimeric receptors and of 32D-IL-2Rβ cells expressing the G/IL-2Rβ, G2R-1 and G2R-2 chimeric receptors.

### Example 14: G-CSFR ECD is Expressed on the Surface of Cells Transduced With G/IL-2Rβ, G2R-1 and G2R-2

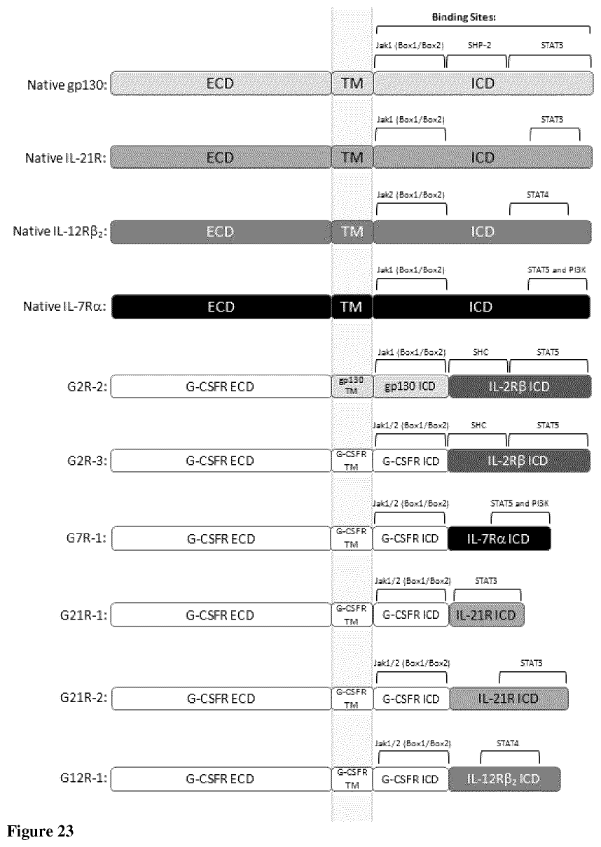

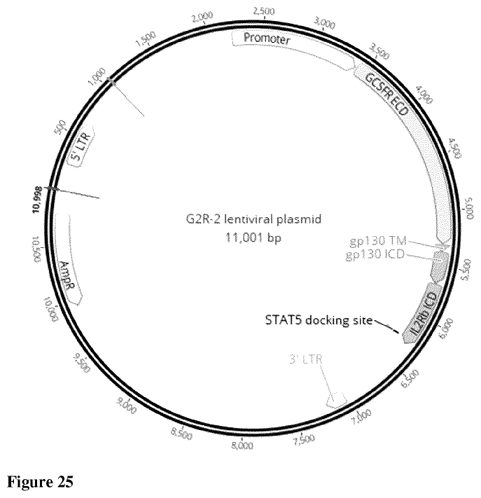

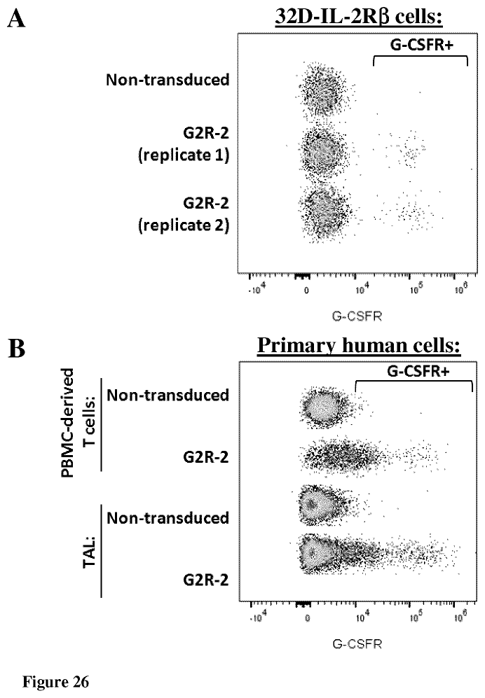

Flow cytometry was performed on the 32D-IL-2Rβ cell line, PBMC-derived human T cells and human tumor-associated lymphocytes after transduction with a lentiviral vector encoding the G2R-2 chimeric cytokine receptor (shown schematically in FIGS. 23 and 25) to determine if the cells express the G-CSFR ECD on the cell surface. G-CSFR positive cells were detected in all transduced cell types (FIG. 26). In a separate experiment, 32D-IL-2Rβ cells expressing the G/IL-2Rβ, G2R-1 and G2R-2 chimeric receptors were positive for the G-CSFR ECD by flow cytometry (lower panels in FIG. 21B-D).

These results indicate that the GU-2Rβ, G2R-1 and G2R-2 chimeric receptors are expressed on the cell surface.

### Example 15: Expansion of Cells Expressing G2R-2 Compared to Non-Transduced Cells

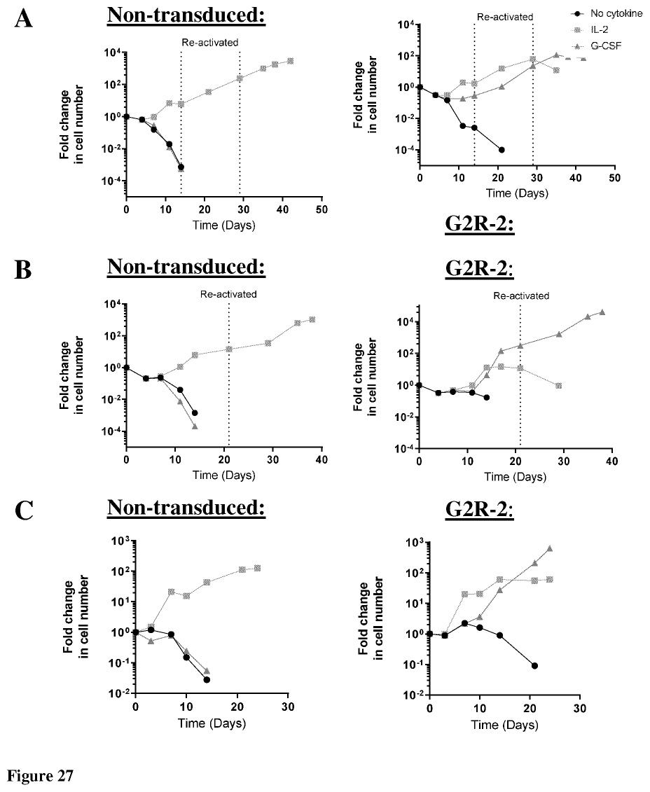

Human PBMC-derived T cells and human tumor-associated lymphocytes were lentivirally transduced with the G2R-2 receptor construct (FIGS. 4 and 6), washed, and re-plated with the indicated cytokine. In some experiments, T cells were also re-activated periodically by stimulation with TransAct. Live cells were counted every 3-4 days. Proliferation of the PMBC-derived T cells (FIG. 27A) and tumor-associated lymphocytes (two independent experiments in FIG. 27B,C) was observed after stimulation with G-CSF (100 ng/ml) in cells expressing the G2R-2 chimeric receptor but not in non-transduced cells.

These results show that G-CSF-induced activation of the G2R-2 chimeric receptor is sufficient to induce proliferation and viability of immune cells.

### Example 16: Expansion and Immunophenotype of CD4- or CD8-Selected Human Tumor-Associated Lymphocytes Expressing G2R-2 Compared to Non-Transduced Cells

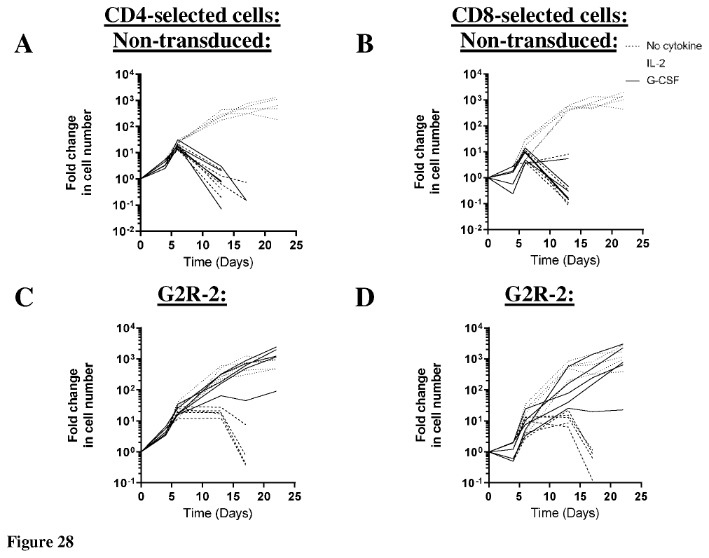

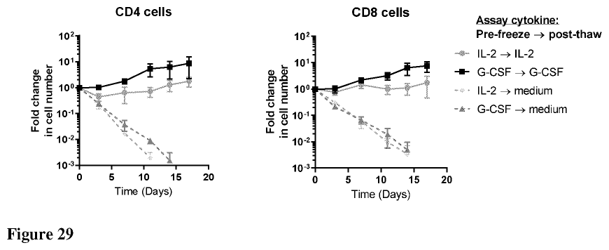

CD4-selected and CD8-selected human T cells were transduced with lentiviral vector encoding G2R-2 (FIG. 25), or left non-transduced where indicated. Cells were washed and re-plated with the indicated cytokine and counted every 3-4 days. Proliferation of CD4- or CD8-selected TALs expressing G2R-2 was observed after stimulation with G-CSF (100 ng/ml) or IL-2 (300 IU/ml), but not in the absence of added cytokine (medium alone) (FIGS. 28 and 29). In FIG. 28, each line represents results from one of 5 patient samples.

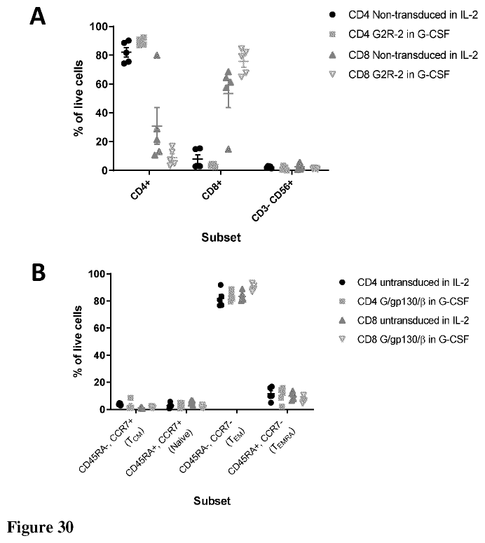

Immunophenotyping by flow cytometry demonstrated that T cells cultured in G-CSF or IL-2 retained their CD4+ or CD8+ identity (FIG. 30A), lacked an NK cell phenotype (CD3-CD56+) (FIG. 30A), and exhibited a CD45RA-CCR7-T effector memory (T_{EM}) phenotype under these culture conditions (FIG. 30B).

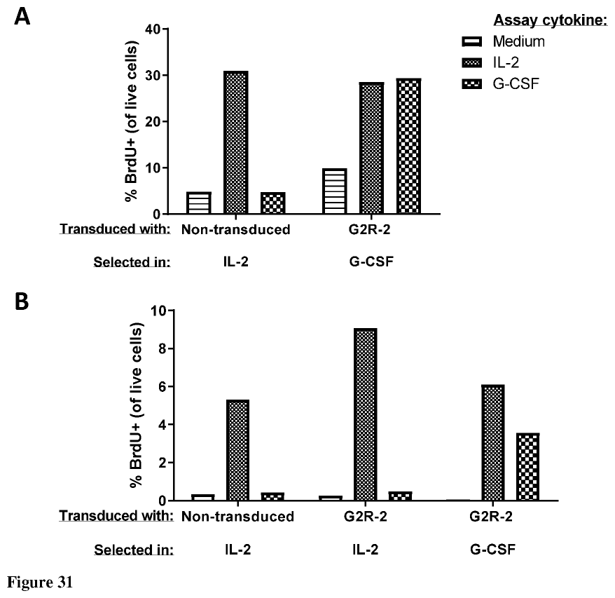

BrdU assays were performed to confirm increased cell cycle progression of T cells expressing G2R-2 upon stimulation with G-CSF (FIG. 31). T cells were selected by culture in IL-2 or G-CSF, as indicated, prior to the assay. Both tumor-associated lymphocytes (FIG. 31A) and PBMC-derived T cells (FIG. 31B) were assessed.

These results show that G-CSF can selectively activate cell cycle progression and long-term expansion of primary human TALs by activation of the chimeric cytokine receptor G2R-2. These results also indicate that activation of the G2R-2 chimeric receptor by homodimer formation is sufficient to activate cytokine-like signaling and proliferation in TALs. Furthermore, TALs expressing G2R-2 remain cytokine dependent in that they undergo cell death upon withdrawal of G-CSF, similar to the response to IL-2 withdrawal. TALs cultured in G-CSF maintain a similar immunophenotype as TALs cultured in IL-2.

### Example 17: Primary Murine T Cells Expressing G2R-2 Proliferate in Response to G-CSF

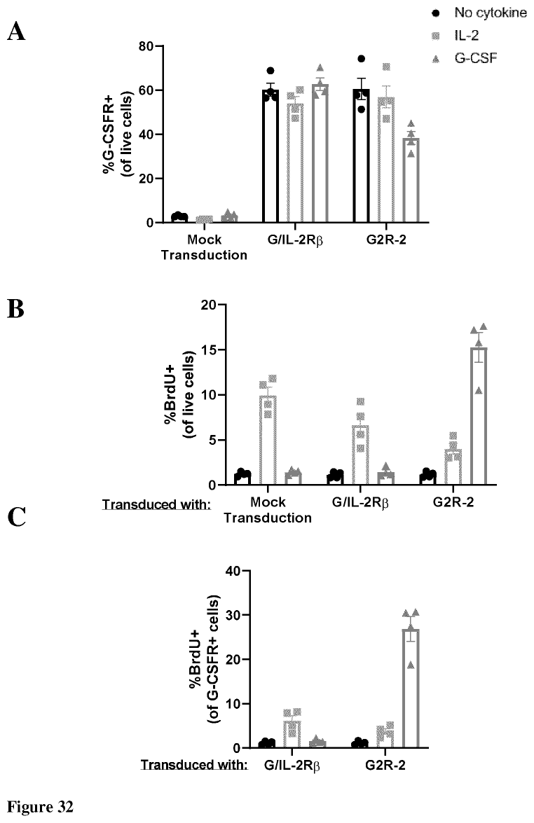

BrdU incorporation assays were performed to assess proliferation of primary murine T cells expressing G2R-2 or the single-chain G/IL-2Rβ (a component of G2R-1) versus mock-transduced cells upon stimulation with G-CSF. All cells were expanded in IL-2 for 3 days prior to assay. Cell surface expression of G2R-2 or G/IL-2Rβ was confirmed by flow cytometry (FIG. 32A). As indicated, cells were then plated in IL-2 (300 IU/ml), wildtype G-CSF (100 ng/ml) or no cytokine. Increased cell cycle progression after stimulation with G-CSF was observed in the cells expressing G2R-2 above that of non-transduced cells or cells expressing the single-chain G/IL-2Rβ (FIGS. 32B,C). Panels B and C show results for all live cells or G-CSFR+ cells, respectively.

These results indicate that, in response to G-CSF-induced homodimerization, the G2R-2 chimeric receptor is more efficient than the single chain G/IL-2Rβ receptor to activate cytokine-like signaling and proliferation in murine T cells.

### Example 18: Activation of Cytokine-Associated Intracellular Signaling Events in of Human Primary T Cells Expressing G2R-2 in Response to G-CSF or IL-2

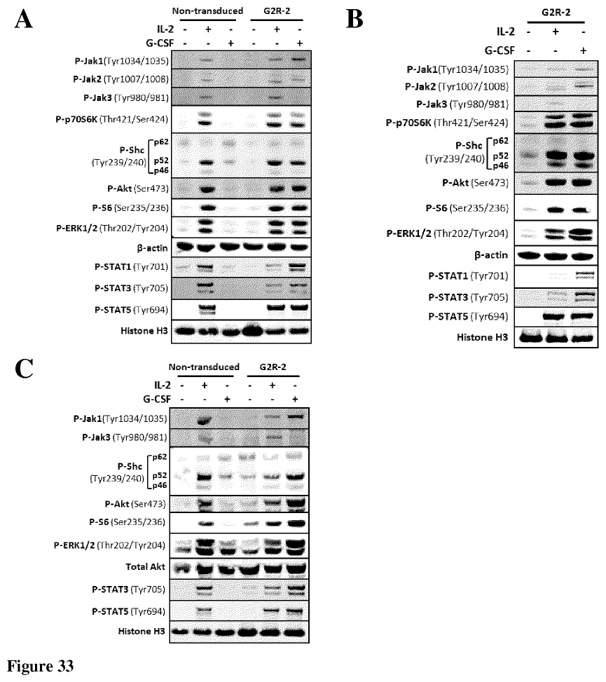

To confirm that the chimeric cytokine receptors were indeed capable of activating cytokine signaling similar to that of IL-2, the ability of the cytokine receptors to activate various signaling molecules was assessed. Tumor-associated lymphocytes and PBMC-derived T cells expressing G2R-2 were previously expanded in G-CSF, while non-transduced cells were previously expanded in IL-2. The cells were washed and then stimulated with IL-2 (300 IU/ml), wildtype G-CSF (100 ng/ml), or no cytokine, and western blots were performed on the cell lysates using antibodies directed against the indicated signaling molecules (FIG. 33). Panels A and B show results for TAL, and panel C for PBMC-derived T cells. T cells expressing G2R-2 activated IL-2-related signaling molecules upon stimulation with G-CSF to a similar extent as seen after IL-2 stimulation of non-transduced cells or transduced cells, with the expected exception that G-CSF induced Jak2 phosphorylation whereas IL-2 induced Jak3 phosphorylation.

These results confirm that the G2R-2 chimeric receptor is capable of activating IL-2 receptor-like cytokine receptor signaling upon stimulation with G-CSF.

### Example 19: Cytokine Signaling is Activated in Response to G-CSF in Murine Primary T Cells Expressing G2R-2

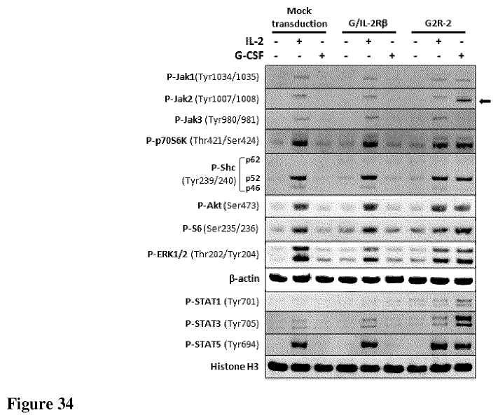

To assess whether the chimeric cytokine receptor G2R-2 or the single-chain G/IL-2Rβ (from G2R-1) were capable of activating cytokine signaling, the ability of these cytokine receptors to activate various signaling molecules was assessed by western blot of cell lysates of murine primary T cells expressing G2R-2 or G/IL-2Rβ versus mock-transduced cells. All cells were expanded in IL-2 for 3 days prior to assay. Cells were then washed and stimulated with IL-2 (300 IU/ml), wildtype G-CSF (100 ng/ml) or no cytokine. The cells expressing G2R-2 activated IL-2-related signaling molecules upon stimulation with G-CSF to a similar extent as seen after IL-2 stimulation of non-transduced cells or transduced cells, with the expected exception that G-CSF induced Jak2 phosphorylation whereas IL-2 induced Jak3 phosphorylation (FIG. 34). In contrast, G/IL-2Rβ did not activate cytokine signaling upon exposure to G-CSF.

These results confirm in primary murine T cells that the G2R-2 chimeric receptor is capable of activating IL-2 receptor-like cytokine receptor signaling by homodimerization upon G-CSF stimulation, whereas the single-chain G/IL-2Rβ alone is not capable of activating cytokine signaling by homodimerization in response to G-CSF.

### Example 20: Expression of Chimeric Receptors Leads to Proliferation of 32D-IL-2Rβ Cells and Primary Murine T Cells after Stimulation with an Orthogonal G-CSF

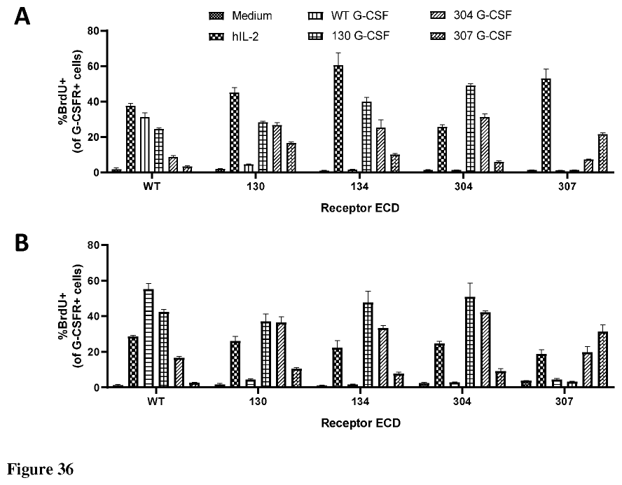

To determine if cells expressing chimeric cytokine receptors could be selectively activated in response to an orthogonal version of G-CSF, 32D-IL-2Rβ cells or primary murine T cells were transduced with the chimeric receptors G2R-1 and G2R-2 that comprises the wild-type G-CSFR ECD (G2R-1 WT ECD, G2R-2 WT ECD) and chimeric receptors G2R-1 and G2R-2 that comprises the G-CSFR ECD which harbors the amino acid substitutions R41E, R141E and R167D (G2R-1 134 ECD, G2R-2 134 ECD). Cells were stimulated with either IL-2, wild type G-CSF or the orthogonal G-CSF (130 G-CSF) capable of binding to G2R-1 134 ECD and G2R-2 134 ECD, but with significantly reduced binding to wild-type G-CSFR. BrdU incorporation assays were performed to assess the ability of the cells to promote cell cycle progression upon cytokine stimulation (FIG. 3). 32D-IL-2Rβ cells expressing G2R-2 134 ECD demonstrated cell cycle progression upon stimulation with 130 G-CSF (harboring amino acid substitutions E46R, L108K, and D112R; 30 ng/ml), but did not undergo cell cycle progression upon stimulation with wild-type G-CSF (30 ng/ml). The orthogonal nature of engineered cytokine:receptor ECD pairs was further demonstrated by stimulating primary murine T cells in a “criss-cross” proliferation assay, where cells expressing G2R-3 (FIG. 23) with the WT, 130, 134, 304 or 307 ECD were stimulated with WT, 130, 304 or 307 cytokine (100 ng/ml) (FIG. 36). The 130 ECD harbors the amino acid substitutions: R41E, and R167D. The 304 ECD harbors the amino acid substitutions: R41E, E93K and R167D; whereas the 304 cytokine harbors the amino acid substitutions: E46R, L108K, D112R and R147E. The 307 ECD harbors the amino acid substitutions: R41E, D197K, D200K and R288E; whereas the 307 cytokine harbors the amino acid substitutions: S12E, K16D, E19K and E46R. Panels A and B in FIG. 36 represent experimental replicates.

These results demonstrate that cells expressing an orthogonal chimeric cytokine receptor are capable of selective activation and cell cycle progression upon stimulation with an orthogonal G-CSF CSF.

### Example 21: Intracellular Signaling is Activated in 32D-IL2Rβ Cells and Primary Human T Cells Expressing Orthogonal Chimeric Cytokine Receptors and Stimulated with an Orthogonal G-CSF

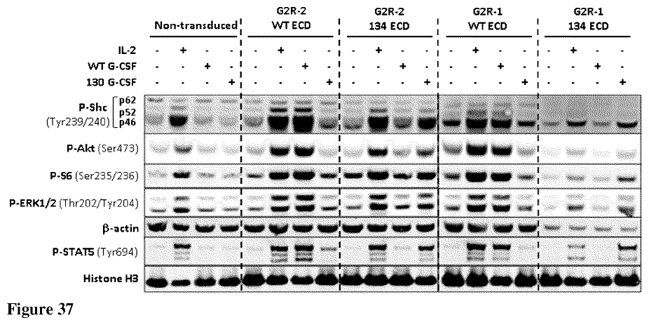

To determine if cells expressing chimeric cytokine receptors could selectively activate intracellular cytokine signaling events in response to an orthogonal version of G-CSF, 32D-IL-2Rβ cells were transduced with the chimeric receptors G2R-1 and G2R-2 that comprises the wild-type G-CSFR ECD (G2R-1 WT ECD and G2R-2 WT ECD) and chimeric receptors G2R-1 and G2R-2 that comprises the G-CSFR ECD which harbors the amino acid substitutions R41E, R141E and R167D (G2R-1 134 ECD, G2R-2 134 ECD). Cells were stimulated with either IL-2 (300 IU/ml), wild type G-CSF (30 ng/ml) or the orthogonal G-CSF (130 G-CSF-E46R_L108K_D112R; 30 ng/ml) capable of binding to G2R-1 134 ECD, G2R-2 134 ECD, but with significantly reduced binding to wild-type G-CSFR. Western blots were performed on cell lysates to assess the ability of the cells to activate cytokine signaling upon exposure to cytokines (FIG. 37). Cells expressing G2R-2 134 ECD showed evidence of cytokine signaling upon stimulation with 130 G-CSF but not wild-type G-CSF. Furthermore, cells expressing G2R-2 WT ECD were not able to activate cytokine signaling upon stimulation with 130 G-CSF.

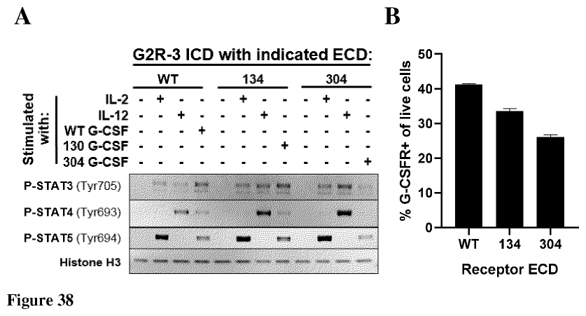

The orthogonal nature of engineered cytokine:receptor pairs was further demonstrated by subjecting primary murine T cells to western blot analysis, where cells expressing G2R-3 with the WT, 134, or 304 ECD (R41E_E93K_R167D) stimulated with WT, 130, or 304 G-CSF (E46R_L108K_D112R_R147E; 100 ng/ml) and the indicated signaling events were measured (FIG. 38A). IL-2 (300 IU/ml) and IL-12 (10 ng/ml) served as control cytokines. Cells expressing G2R-3 WT ECD showed evidence of cytokine signaling upon stimulation with IL-2, IL-12 or WT G-CSF. Cells expressing G2R-3 134 ECD showed evidence of cytokine signaling upon stimulation with IL-2, IL-12 or 130 G-CSF. Cells expressing G2R-3 304 ECD showed evidence of cytokine signaling upon stimulation with IL-2, IL-12 or 304 G-CSF.

Cell surface expression of the three ECD variants of G2R-3 was confirmed by flow cytometry (FIG. 38B).

These results demonstrate that cells expressing an orthogonal chimeric cytokine receptor are capable of selective activation of intracellular cytokine signaling events upon stimulation with an orthogonal G-CSF.

### Example 22: Expression of G2R-3 Leads to Expansion, Cell Cycle Progression and Cytokine-Related Intracellular Signaling and Immunophenotype in Primary Human T Cells

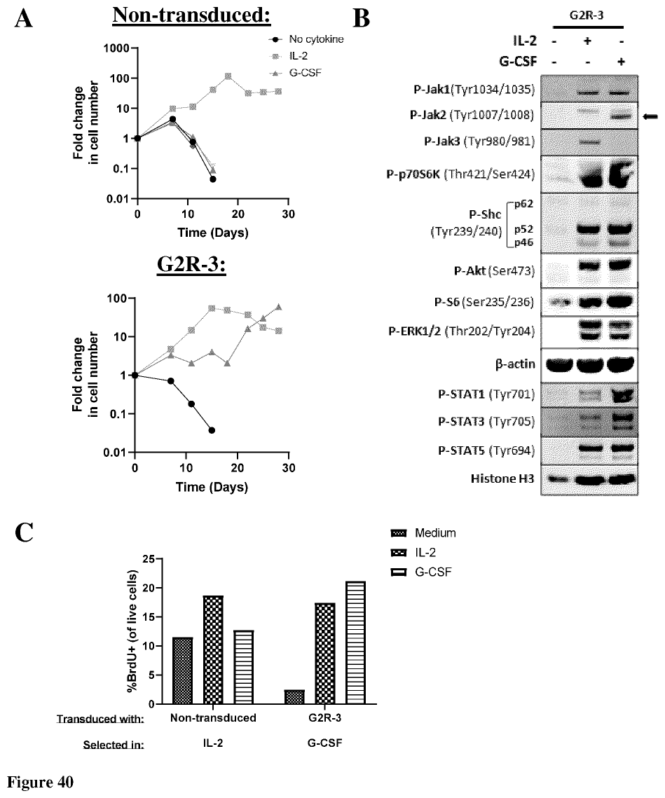

To determine whether the G2R-3 chimeric receptor can promote cytokine signaling-related events upon stimulation with G-CSF in primary human T cells, TALs were transduced with a lentiviral vector encoding G2R-3. T-cell expansion assays were performed to test the proliferation of cells upon stimulation with IL-2 (300 IU/ml), wildtype G-CSF (100 ng/ml) or no cytokine. Live cells were counted every 3-4 days. In contrast to their non-transduced counterparts, primary TALs expressing G2R-3 expanded in culture in response to G-CSF (FIG. 40A). To determine whether cytokine signaling events were activated upon stimulation with G-CSF, western blots were performed on cell lysates to assess intracellular signaling. Cells were harvested from the expansion assay, washed, and stimulated with IL-2 (300 IU/ml) or wildtype G-CSF (100 ng/ml). Primary TALs expressing G2R-3 demonstrated IL-2-related signaling events in response to G-CSF, with the expected exception that G-CSF induced Jak2 phosphorylation whereas IL-2 induced Jak3 phosphorylation (FIG. 40B).

BrdU incorporation assays were performed to assess cell cycle progression upon stimulation with G-CSF. Cells were harvested from the expansion assay, washed, and re-plated in IL-2 (300 IU/ml), wildtype G-CSF (100 ng/ml) or no cytokine. Primary TALs expressing G2R-3 demonstrated cell cycle progression in response to G-CSF (FIG. 40C).

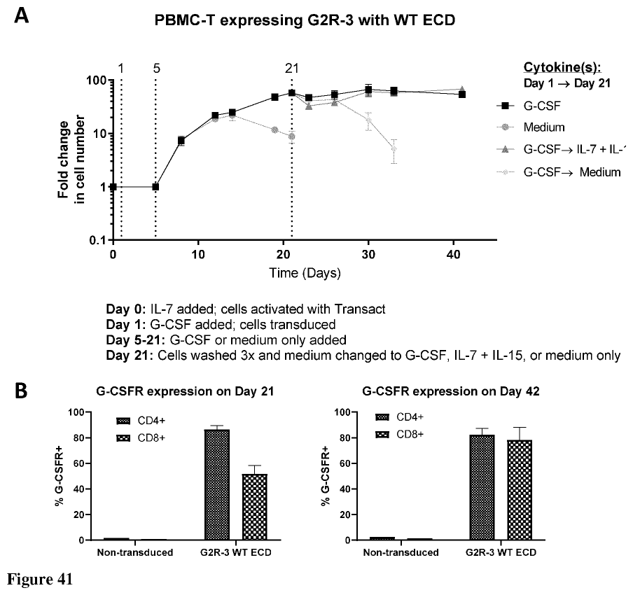

G-CSF-induced expansion of cells expressing G2R-3 was also demonstrated using primary PBMC-derived human T cells (FIG. 41). Cells expressing G2R-3 WT ECD expanded in response to WT G-CSF but not medium alone (FIG. 41A). To demonstrate the continued dependence of cells on exogenous cytokine, on Day 21 of culture, cells from the G-CSF-expanded condition were washed and re-plated in WT G-CSF (100 ng/mL), IL-7 (20 ng/mL)+IL-15 (20 ng/mL), or medium only. Only cells re-plated in the presence of G-CSF or IL-7+IL-15 remained viable over time.

Expression of G-CSFR ECD, as assessed by flow cytometry, remained stable on both CD4+ and CD8+ T cells between days 21-42 of expansion (FIG. 41B).

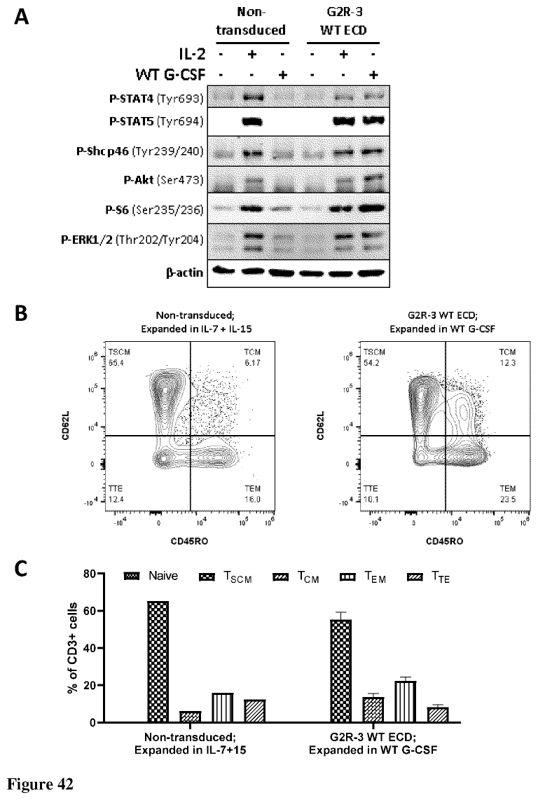

By western blot, primary PBMC-derived T cells expressing G2R-3 demonstrated IL-2-related signaling events in response to G-CSF (FIG. 42A). Flow cytometry-based immunophenotyping was performed on primary PBMC-derived T cells expanded for 42 days in WT G-CSF versus IL-7+IL-15. Cells expressing G2R-3 WT ECD and cultured in G-CSF retained a similar phenotype to non-transduced cells cultured in IL-7+IL-15, with primarily a CD62L+, CD45RO+ phenotype, which is indicative of a stem cell-like memory T cell phenotype (T_{SCM}) (FIG. 42B, C). Likewise, the fractions of central memory (T_{CM}), effector memory (T_{EM}), and terminally differentiated (T_{TE}) T cells were similar.

These results confirm that the G2R-3 chimeric cytokine receptor is capable of activating cytokine signaling events and promoting cell cycle progression and expansion in primary cells. The immunophenotype of T cells expressing G2R-3 and expanded long-term in G-CSF is similar to non-transduced cells expanded in IL-7+IL-15.

### Example 23: Orthogonal G-CSF Induces Expansion and Proliferation in Primary Human T Cells Expressing G2R-3 with Orthogonal ECD

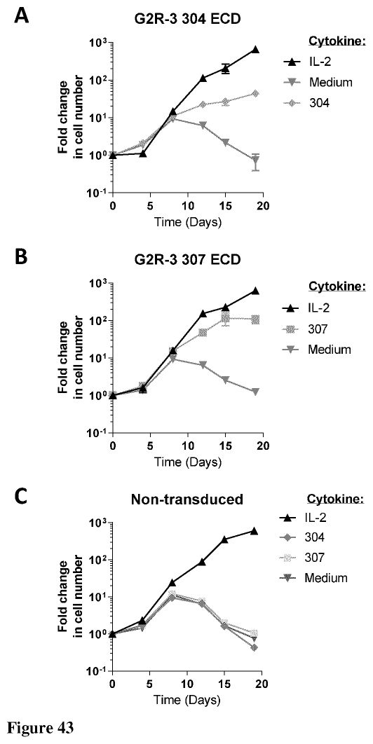

We assessed whether the chimeric cytokine receptor G2R-3 with 304 (R41E_E93K_R167D) or 307 (R41E_D197K_D200K_R288E) ECD was capable of inducing proliferation and expansion in response to stimulation with the orthogonal ligand 130, 304 or 307 G-CSF, primary PBMC-derived human T cells were transduced with lentiviral vectors encoding GR-3 304 ECD or G2R-3 307 (R41E_D197K_D200K_R288E) ECD. T-cell growth assays were performed to assess the fold expansion of cells when cultured with IL-2 (300 IU/ml), 304 G-CSF (100 ng/ml), 307 G-CSF (100 ng/ml) or no cytokine. Live cells were counted every 3-4 days. T cells expressing G2R-3 304 ECD expanded in culture in response to IL-2 or 304 G-CSF (FIG. 43A). T cells expressing G2R-3 307 ECD expanded in culture in response to IL-2 or 307 G-CSF (FIG. 43B). Non-transduced T cells only expanded in response to IL-2 (FIG. 43C).

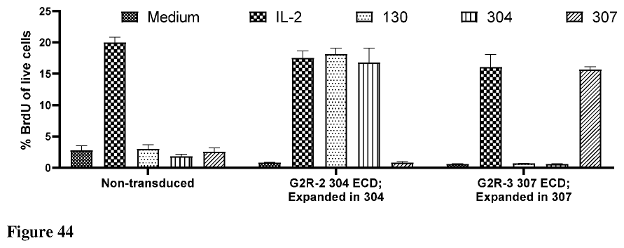

BrdU incorporation assays were performed to assess cell cycle progression upon stimulation with 130, 304 and 307 G-CSF in a criss-cross design. Cells were harvested from the expansion assay, washed, and re-plated in IL-2 (300 IU/ml), 130 G-CSF (100 ng/ml), 304 G-CSF (100 ng/ml), 307 G-CSF (100 ng/ml) or no cytokine. Primary human T cells expressing G2R-3 304 ECD demonstrated cell cycle progression in response to 130 or 304 G-CSF, but not in response to 307 G-CSF (FIG. 44). T cells expressing G2R-3 307 ECD demonstrated cell cycle progression in response to 307 G-CSF, but not in response to 130 or 304 G-CSF. All T cells demonstrated cell cycle progression in response to IL-2.

The results show that the chimeric receptors G2R-3 304 ECD and G2R-3 307 ECD are capable of inducing selective cell cycle progression and expansion of primary human CD4+ and CD8+ T cells upon stimulation with orthogonal 304 or 307 G-CSF, respectively. Furthermore, 130 G-CSF can stimulate proliferation of cells expressing G2R-3 304 ECD, but not G2R-3 307 ECD.

### Example 24: G-CSFR ECD is Expressed on the Surface of Primary Human Tumor-Associated Lymphocytes (Tal) Transduced with G21R-1, G21R-2, G12R-1 and G2R-3 Chimeric Receptor Constructs

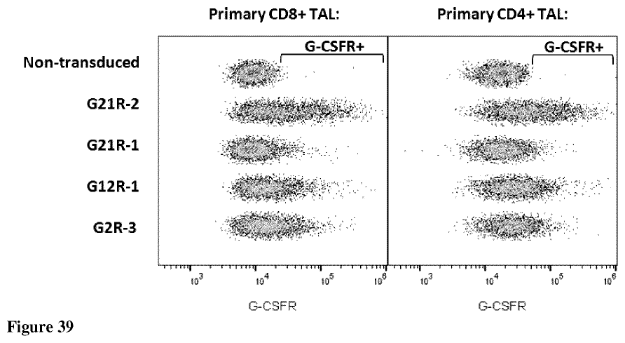

To assess whether chimeric cytokine receptor constructs can be expressed on the surface of primary human tumor-associated lymphocytes (TAL), TAL were transduced with lentiviral vectors encoding the G21R-1, G21R-2, G12R-1 and G2R-3 chimeric receptors, and the cells were tested by flow cytometry for G-CSFR ECD expression on the cell surface (FIG. 39). For all four chimeric cytokine receptor designs, G-CSFR ECD positive cells were detected.

These results demonstrate that the G21R-1, G21R-2, G12R-1 and G2R-3 chimeric receptors are capable of being expressed on the surface of primary cells. These results also indicate that G-CSFR ECD chimeric receptor designs are expressed on the surface of primary cells.

### Example 25: G-CSFR ECD is Expressed on the Surface of Primary Murine T Cells Transduced with G12R-1 and G21R-1 Chimeric Receptor Constructs

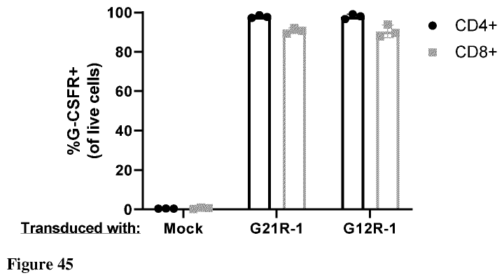

To determine if the G12R-1 and G21R-1 chimeric receptors are capable of being expressed on the surface of primary T cells, primary murine T cells were transduced with retroviral vectors encoding the G12R-1 and G21R-1 chimeric receptors and analyzed by flow cytometry (FIG. 45).

The results show that G-CSFR ECD is expressed on the surface of primary murine CD4+ and CD8+ T cells transduced with retroviral vectors encoding G12R-1 and G21R-1.

### Example 26: G-CSF Induces Cytokine Signaling Events in Primary PBMC-Derived Human T Cells Expressing G21R-1 or G21R-2

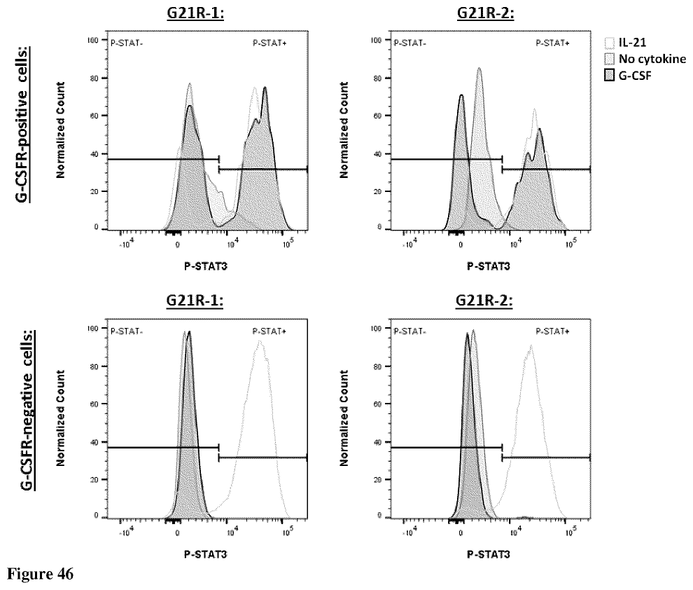

To determine whether the G21R-1 and G21R-2 constructs are capable of inducing cytokine signaling events in primary cells, primary PBMC-derived human T cells were transduced with lentiviral vectors encoding G21R-1 or G21R-2 chimeric cytokine receptors. Cells were subjected to intracellular staining with phospho-STAT3 (p-STAT3) specific antibody and assessed by flow cytometry to determine the extent of STAT3 phosphorylation, a measure of STAT3 activation (FIG. 46). Upon stimulation with G-CSF (100 ng/ml), the number of cells expressing phosphorylated STAT3 increased among the subset of G-CSFR positive cells transduced with either G21R-1 or G21R-2. In contrast, the G-CSFR negative (i.e. non-expressing) cells did not exhibit an increase in phosphorylated STAT3 upon stimulation with G-CSF but did upon stimulation with IL-21.

These results demonstrate that the G21R-1 and G21R-2 chimeric receptors are capable of activating IL-21-related cytokine signaling events upon stimulation with G-CSF in primary human T cells.

### Example 27: G-CSF Induces Intracellular Signaling Events in Primary Murine T Cells Expressing G21R-1 or G-12R-1

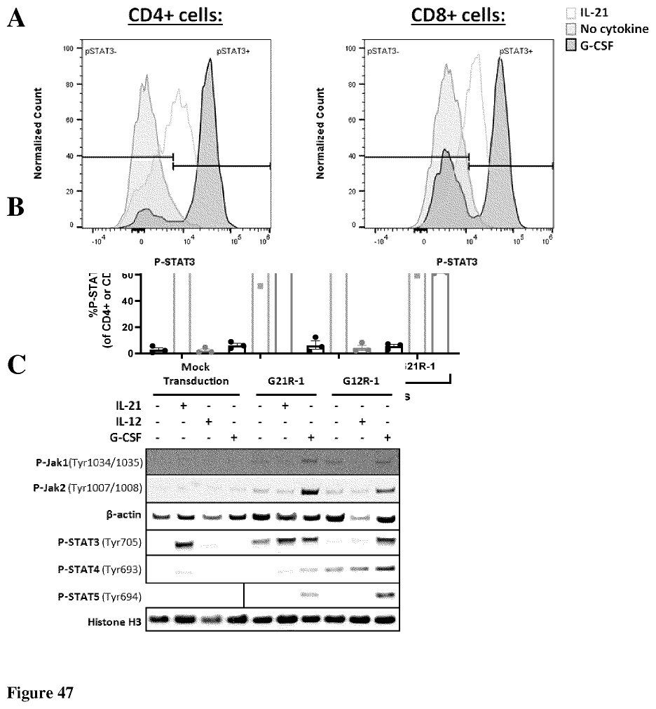

To determine if the chimeric cytokine receptor G21R-1 was capable of activating cytokine signaling events, primary murine T cells were transduced with a retroviral vector encoding G21R-1 and assessed by flow cytometry to detect phosphorylated STAT3 upon stimulation with G-CSF. Live cells were gated on CD8 or CD4, and the percentage of cells staining positive for phospho-STAT3 after stimulation with no cytokine, IL-21 (1 ng/ml) or G-CSF (100 ng/ml) was determined for the CD8 and CD4 cell populations. Upon stimulation with G-CSF, cells expressing G21R-1 (but not non-transduced cells) showed increased amounts of phosphorylated STAT3 (FIGS. 47A and 47B). Western blots were performed to assess intracellular cytokine signaling in cells expressing G21R-1 or G12R-1 upon stimulation with G-CSF. As expected, cells expressing G21R-1 and stimulated with G-CSF showed increased phosphorylation of STAT3, with minor increases in phospho-STAT4 and phospho-STAT5 (FIG. 47C). Also as expected, in cells expressing G12R-1, strong phosphorylation of STAT4 was seen in response to G-CSF. G-CSF did not induce any signaling events in mock transduced cells. (Note that in the G12R-1 group, the positive control (hIL-12 10 ng/ml) did not appear to induce any signaling events; this may be due to poor binding of human IL-12 to the murine IL-12R.)

The results show that G21R-1 and G12R-1 are capable of inducing cytokine signaling events in primary murine T cells upon stimulation with G-CSF.

### Example 28: G-CSF Induces Proliferation and Intracellular Signaling Events in Primary Murine T Cells Expressing G2R-2, G2R-3, G7R-1, G21/7R-1, G27/2R-1, G21/2R-1, G12/2R-1 OR G21/12/2R-1

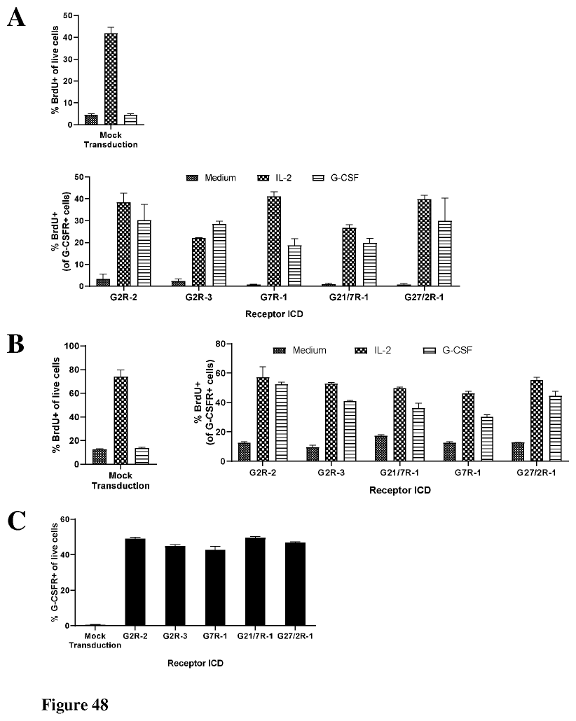

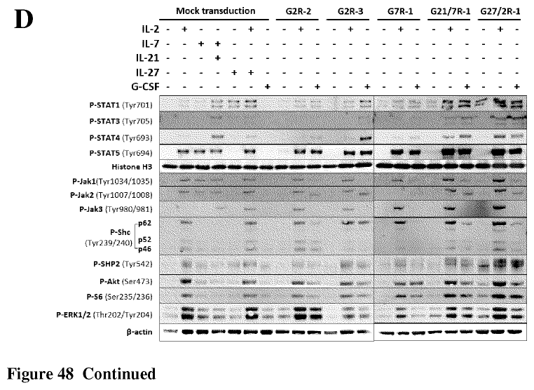

To assess cytokine signaling events and cell proliferation mediated by chimeric cytokine receptors, primary murine T cells were transduced with retroviral vectors encoding G2R-2, G2R-3, G7R-1, G21/7R-1, G27/2R-1, G21/2R-1, G12/2R-1 or G21/12/2R-1. BrdU incorporation assays were performed to assess cell cycle progression upon stimulation with G-CSF. Cells were harvested, washed, and re-plated in IL-2 (300 IU/ml), wildtype G-CSF (100 ng/ml) or no cytokine. G-CSF-induced-cell cycle progression was seen in primary murine T cells expressing G2R-2, G2R-3, G7R-1, G21/7R-1 or G27/2R-1 (FIGS. 48A, 48B) or, G21/2R-1, G12/2R-1 or G21/12/2R-1 (FIGS. 49A, 49B). Expression of the G-CSFR ECD was also detectable by flow cytometry (FIGS. 48C, 49C).

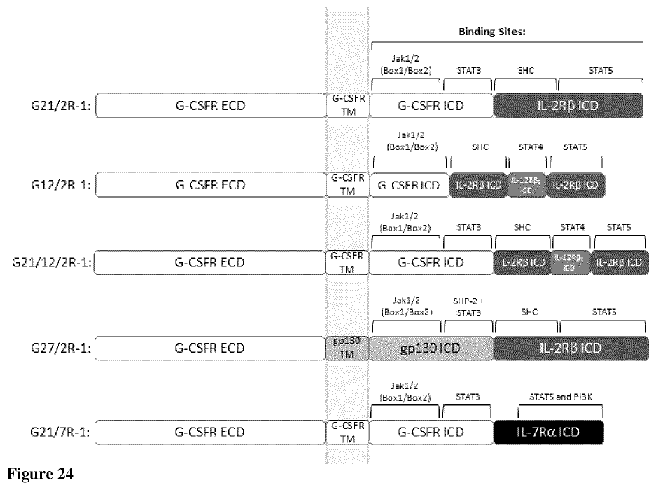

By Western blot, multiple cytokine signaling events were observed in response to G-CSF (100 ng/ml) in cells expressing the indicated chimeric cytokine receptors, but not in mock transduced cells (FIGS. 48D, 49D). In general, the observed cytokine signaling events were as expected based on the signaling domains that were incorporated into the various ICD designs (FIGS. 23 and 24). As one example, the G7R-1 chimeric receptor induced phosphorylation of STAT5 (FIG. 48D), which is expected due to the incorporation of the STAT5 binding site from IL-7Rα (FIG. 23). As a second example, the G21/2R-1 chimeric receptor induced phosphorylation of STAT3 (FIG. 49D), which is expected due to the incorporation of the STAT3 binding site from G-CSFR (FIG. 24). As a third example, the G12/2R-1 chimeric receptor induced phosphorylation of STAT4, which is expected due to the incorporation of the STAT4 binding site from IL-12Rβ2 (FIG. 24). Other chimeric cytokine receptors showed other distinct patterns of intracellular signaling events.

The results show that G2R-2, G2R-3, G7R-1, G21/7R-1, G27/2R-1, G21/2R-1, G12/2R-1 and G21/12/2R-1 are capable of inducing cytokine signaling events and proliferation in primary murine T cells upon stimulation with G-CSF. Furthermore, different patterns of intracellular signaling events can be generated by incorporating different signaling domains into the ICD of the chimeric receptor.

### Example 29: Orthogonal G-CSF Induces Expansion, Proliferation Cytokine Related Intracellular Signaling and Immunophenotype in Primary Human T Cells Expressing G12/2R-1 with Orthogonal ECD

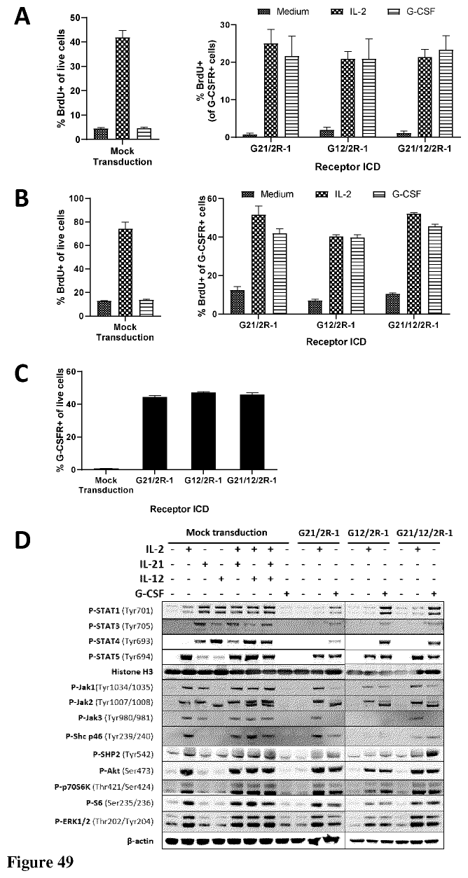

To determine if the chimeric cytokine receptor G12/2R-1 with 134 ECD was capable of inducing proliferation and expansion in response to stimulation with the orthogonal ligand 130 G-CSF, primary PBMC-derived human T cells were transduced with a lentiviral vector encoding G12/2R-1 134 ECD. T-cell growth assays were performed to assess the fold expansion of cells when cultured with IL-2 (300 IU/ml), 130 G-CSF (100 ng/ml) or no cytokine. Live cells were counted every 4-5 days. Primary human T cells expressing G12/2R-1 134 ECD expanded in culture in response to IL-2 or 130 G-CSF (FIG. 50A) but showed limited, transient expansion in medium alone.

On day 19 of this experiment, T cells that had been expanded in 130 G-CSF or IL-2 were washed three times are re-plated in IL-2, 130 G-CSF or medium alone. In medium alone, T cells showed reduced viability and a decline in number (FIG. 50B). In contrast, T cells re-plated in IL-2 or G-CSF 130 showed continued viability and stable numbers.

Expression of the G12/2R-1 134 ECD, detected by flow cytometry with an antibody against the G-CSF receptor, increased between Day 4 and Day 16 on both CD4+ and CD8+ T cells expanded by stimulation with 130 G-CSF (FIG. 50C). BrdU incorporation assays were performed to assess cell cycle progression upon stimulation with 130 G-CSF.

To assess cell cycle progression by BrdU assay, cells were harvested from the expansion assay, washed, and re-plated in IL-2 (300 IU/ml), IL-2 and IL-12 (long/mL), 130 G-CSF (300 ng/ml) or no cytokine. Primary human T cells expressing G12/2R-1 134 ECD demonstrated cell cycle progression in response to 130 G-CSF, IL-2 or IL-2+IL-12, whereas non-transduced cells responded only to IL-2 or IL-2+IL-12 (Figure MA).

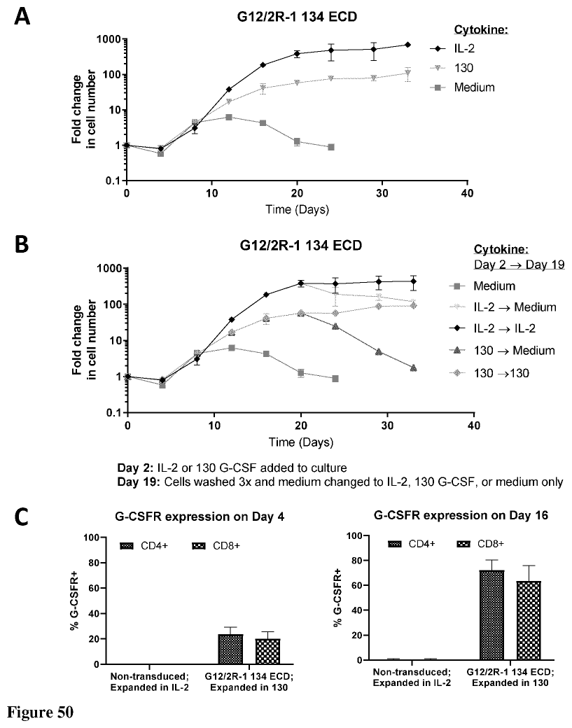

After a 16 day culture period, immunophenotyping was performed by flow cytometry with antibodies to CD62L and CD45RO to compare T cells expressing G12/2R-1 134 ECD expanded in 130 G-CSF to non-transduced cells expanded in IL-2. The two T cell populations showed similar proportions of stem cell-like memory (T_{SCM}), central memory (T_{CM}), effector memory (T_{EM}), and terminally differentiated (T_{TE}) phenotypes (FIG. 51B, C).

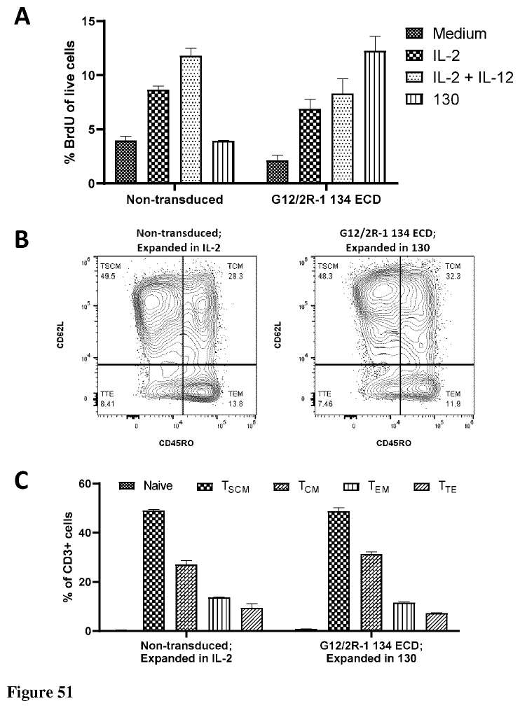

Similar experiments were performed with the chimeric cytokine receptor G12/2R-1 with 304 ECD (as opposed to 134 ECD). Primary PBMC-derived human T cells were transduced with a lentiviral vector encoding G12/2R-1 304 ECD. T-cell growth assays were performed to assess the fold expansion of cells when cultured with IL-2 (300 IU/ml), 130 G-CSF (100 ng/ml), 304 G-CSF (100 ng/mL) or medium alone. Live cells were counted every 4-5 days. T cells expressing G12/2R-1 with 304 ECD could expand in the presence of IL-2, 130 G-CSF or 304 G-CSF, but not in medium alone, while non-transduced cells could expand only in response to IL-2 (FIG. 52A).

To assess cell cycle progression by BrdU assay, T cells expressing G12/2R-1 304 ECD, previously expanded in either 130 G-CSF or 304 G-CSF, were harvested from the expansion assay, washed, and re-plated in IL-2 (300 IU/ml), 130 G-CSF (100 ng/ml), 304 G-CSF (100 ng/mL), 307 G-CSF (100 ng/mL), or medium alone. T cells expressing G12/2R-1 304 ECD demonstrated cell cycle progression in response to 130 or 304 G-CSF, but not in response to 307 G-CSF or medium alone (FIG. 52B).

The results show that G12/2R-1 134 ECD is capable of inducing cell cycle progression and expansion of primary human CD4+ and CD8+ T cells upon stimulation with orthogonal 130 G-CSF. The T cell memory phenotype of cells expressing G12/2R-1 134 ECD and expanded with 130 G-CSF is similar to non-transduced cells expanded with IL-2. In addition, G12/2R-1 304 ECD is capable of inducing selective cell cycle progression and expansion of T cells upon stimulation with 130 or 304 G-CSF, but not in response to 307 G-CSF.

### Example 30: Orthogonal G-CSF Induces Distinct Intracellular Signaling Events in Primary Human T Cells Expressing G2R-3 or G12/2R-1 with Orthogonal ECD

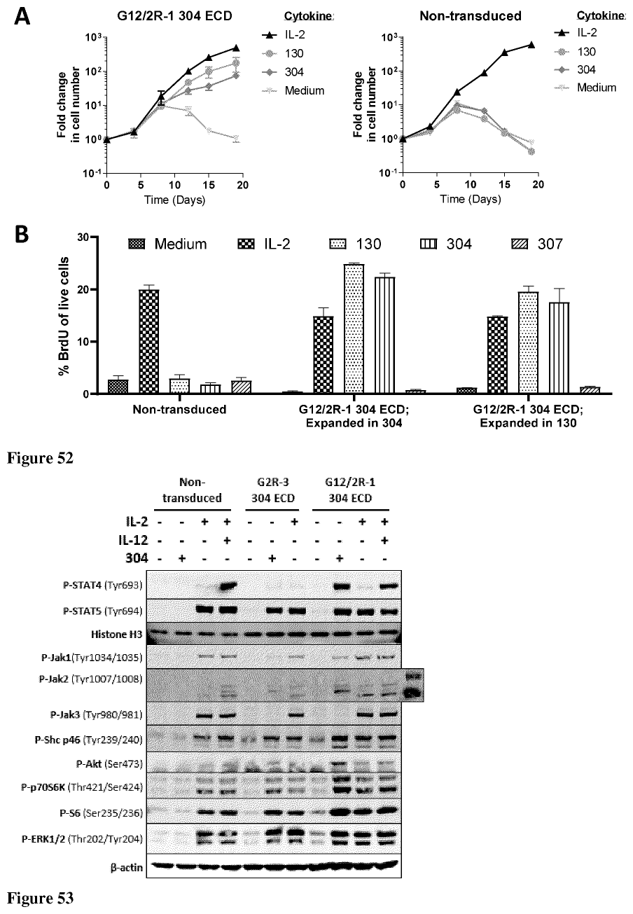

To assess intracellular signaling events, primary PBMC-derived human T cells were transduced with lentiviral vectors encoding G2R-3 304 ECD or G12/2R-1 304 ECD. Western blots were performed to assess intracellular cytokine signaling in cells expressing G2R-3 304 ECD or G12/2R-1 304 ECD, or non-transduced cells, upon stimulation with 304 G-CSF (100 ng/mL), IL-2 (300 IU/mL), IL-2 and IL-12 (10 ng/mL) or medium alone. In both transduced and non-transduced T cells, strong phosphorylation of STAT5 was detected in response to stimulation with either IL-2+IL-12, or IL-2 alone (FIG. 53). Strong phosphorylation of STAT4 was detected in response to stimulation with both IL-2+IL-12, but only weak phosphorylation of STAT4 was detected in response to stimulation with IL-2 alone. In cells expressing G2R-3 304 ECD, weak phosphorylation of STAT4 and strong phosphorylation of STAT5 was detected in response to stimulation with 304 G-CSF, similar to the pattern seen in response to IL-2 alone. In cells expressing G12/2R-1 304 ECD, strong phosphorylation of STAT4 and STAT5 was detected in response to stimulation with 304 G-CSF, similar to the pattern seen in response to IL-2+IL-12. Non-transduced T cells showed no response to 304 G-CSF.

The results show that G12/2R-1 with 304 ECD is capable of inducing cytokine signaling events, including strong phosphorylation of STAT4 and STAT5, in response to stimulation with 304 G-CSF. A different pattern of signaling events is seen in cells expressing G2R-3 304 ECD after stimulation with 304 G-CSF, including strong phosphorylation of STAT5 but not STAT4.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

TABLE 12
Sequence of G-CSFR_{WT}-ICD_{gp130-IL-2Rβ}

| | | | | SEQ |
|---|---|---|---|---|
| | Uniprot | Residue | | ID |
| Protein | ID | numbering | Polypeptide Sequence | NO. |
| G-CSFR | Q99062 | 1-627 | MARLGNCSLTWAALIILLLPGSLEECGHISVS | 3 |
| (Signal | | | APIVHLGDPITASCIIKQNCSHLDPEPQILWRL | |
| peptide, | | | GAELQPGGRQQRLSDGTQESIITLPHLNHTQA | |
| ECD) | | | FLSCCLNWGNSLQILDQVELRAGYPPAIPHNL | |
| | | | SCLMNLTTSSLICQWEPGPETHLPTSFTLKSF | |
| | | | KSRGNCQTQGDSILDCVPKDGQSHCCIPRKH | |
| | | | LLLYQNMGIWVQAENALGTSMSPQLCLDPM | |
| | | | DVVKLEPPMLRTMDPSPEAAPPQAGCLQLC | |
| | | | WEPWQPGLHINQKCELRHKPQRGEASWALV | |
| | | | GPLPLEALQYELCGLLPATAYTLQIRCIRWPL | |
| | | | PGHWSDWSPSLELRTTERAPTVRLDTWWRQ | |
| | | | RQLDPRTVQLFWKPVPLEEDSGRIQGYVVSW | |
| | | | RPSGQAGAILPLCNTTELSCTFHLPSEAQEVA | |
| | | | LVAYNSAGTSRPTPVVFSESRGPALTRLHAM | |
| | | | ARDPHSLWVGWEPPNPWPQGYVIEWGLGPP | |
| | | | SASNSNKTWRMEQNGRATGFLLKENIRPFQL | |
| | | | YEIIVTPLYQDTMGPSQHVYAYSQEMAPSHA | |
| | | | PELHLKHIGKTWAQLEWVPEPPELGKSPLTH | |
| | | | YTIFWTNAQNQSFSAILNASSRGFVLHGLEPA | |
| | | | SLYHIHLMAASQAGATNSTVLTLMTLTPEGS | |
| | | | ELH | |
| gp130 | P40189 | 628-751 | AIVVPVCLAFLLTTLLGVLFCFNKRDLIKKHI | 4 |
| (TM, | | | WPNVPDPSKSHIAQWSPHTPPRHNFNSKDQM | |
| ICD) | | | YSDGNFTDVSVVEIEANDKKPFPEDLKSLDLF | |
| | | | KKEKINTEGHSSGIGGSSCMSSSRPSIS | |
| IL- | P14784 | 752-857 | ASLSSNHSLTSCFTNQGYFFFHLPDALEIEAC | 5 |
| 2RβICD) | | | QVYFTYDPYSEEDPDEGVAGAPTGSSPQPLQ | |
| | | | PLSGEDDAYCTFPSRDDLLLFSPSLLGGPSPPS | |
| | | | TAPGGSGAGEERMPPSLQERVPRDWDPQPLG | |
| | | | PPTPGVPDLVDFQPPPELVLREAGEEVPDAGP | |
| | | | REGVSFPWSRPPGQGEFRALNARLPLNTDAY | |
| | | | LSLQELQGQDPTHLV | |

TABLE 13
Sequence of G-CSFR_{WT}-ICD_{IL-2Rβ}

| | | | | SEQ |
|---|---|---|---|---|
| | Uniprot | Residue | | ID |
| Protein | ID | numbering | Polypeptide Sequence | NO. |
| G-CSFR | Q99062 | 1-627 | MARLGNCSLTWAALIILLLPGSLEECGHISVS | 6 |
| (Signal | | | APIVHLGDPITASCIIKQNCSHLDPEPQILWRL | |
| peptide, | | | GAELQPGGRQQRLSDGTQESIITLPHLNHTQA | |
| ECD) | | | FLSCCLNWGNSLQILDQVELRAGYPPAIPHNL | |
| | | | SCLMNLTTSSLICQWEPGPETHLPTSFTLKSF | |
| | | | KSRGNCQTQGDSILDCVPKDGQSHCCIPRKH | |
| | | | LLLYQNMGIWVQAENALGTSMSPQLCLDPM | |
| | | | DVVKLEPPMLRTMDPSPEAAPPQAGCLQLC | |
| | | | WEPWQPGLHINQKCELRHKPQRGEASWALV | |
| | | | GPLPLEALQYELCGLLPATAYTLQIRCIRWPL | |
| | | | PGHWSDWSPSLELRTTERAPTVRLDTWWRQ | |
| | | | RQLDPRTVQLFWKPVPLEEDSGRIQGYVVSW | |
| | | | RPSGQAGAILPLCNTTELSCTFHLPSEAQEVA | |
| | | | LVAYNSAGTSRPTPVVFSESRGPALTRLHAM | |
| | | | ARDPHSLWVGWEPPNPWPQGYVIEWGLGPP | |
| | | | SASNSNKTWRMEQNGRATGFLLKENIRPFQL | |
| | | | YEIIVTPLYQDTMGPSQHVYAYSQEMAPSHA | |
| | | | PELHLKHIGKTWAQLEWVPEPPELGKSPLTH | |
| | | | YTIFWTNAQNQSFSAILNASSRGFVLHGLEPA | |
| | | | SLYHIHLMAASQAGAINSTVLTLMTLTPEGS | |
| | | | ELH | |
| IL-2Rβ | P14784 | 628-956 | AIVVPVCLAFLLTTLLGVLFCFNKRDLIKKHI | 7 |
| (TM and | | | WPNVPDPSKSHIAQWSPHTPPRHNFNSKDQM | |
| ICD) | | | YSDGNFTDVSVVEIEANDKKPFPEDLKSLDLF | |
| | | | KKEKINTEGHSSGIGGSSCMSSSRPSISASLSS | |
| | | | NHSLTSCFTNQGYFFFHLPDALEIEACQVYFT | |
| | | | YDPYSEEDPDEGVAGAPTGSSPQPLQPLSGED | |
| | | | DAYCTFPSRDDLLLFSPSLLGGPSPPSTAPGGS | |
| | | | GAGEERMPPSLQERVPRDWDPQPLGPPTPGV | |
| | | | PDLVDFQPPPELVLREAGEEVPDAGPREGVSF | |
| | | | PWSRPPGQGEFRALNARLPLNTDAYLSLQEL | |
| | | | QGQDPTHLV | |

TABLE 14
Sequence of G-CSFR_{WT}-ICD_{γc}

| | Uniprot | Residue | | SEQ |
|---|---|---|---|---|
| Protein | ID | numbering | Polypeptide Sequence | ID NO. |
| G-CSFR | Q99062 | 1-627 | MARLGNCSLTWAALIILLLPGSLEECGHISVS | 8 |
| (Signal | | | APIVHLGDPITASCIIKQNCSHLDPEPQILWRL | |
| peptide, | | | GAELQPGGRQQRLSDGTQESIITLPHLNHTQA | |
| ECD) | | | FLSCCLNWGNSLQILDQVELRAGYPPAIPHNL | |
| | | | SCLMNLTTSSLICQWEPGPETHLPTSFTLKSF | |
| | | | KSRGNCQTQGDSILDCVPKDGQSHCCIPRKH | |
| | | | LLLYQNMGIWVQAENALGTSMSPQLCLDPM | |
| | | | DVVKLEPPMLRTMDPSPEAAPPQAGCLQLC | |
| | | | WEPWQPGLHINQKCELRHKPQRGEASWALV | |
| | | | GPLPLEALQYELCGLLPATAYTLQIRCIRWPL | |
| | | | PGHWSDWSPSLELRTTERAPTVRLDTWWRQ | |
| | | | RQLDPRTVQLFWKPVPLEEDSGRIQGYVVSW | |
| | | | RPSGQAGAILPLCNTTELSCTFHLPSEAQEVA | |
| | | | LVAYNSAGTSRPTPVVFSESRGPALTRLHAM | |
| | | | ARDPHSLWVGWEPPNPWPQGYVIEWGLGPP | |
| | | | SASNSNKTWRMEQNGRATGFLLKENIRPFQL | |
| | | | YEIIVTPLYQDTMGPSQHVYAYSQEMAPSHA | |
| | | | PELHLKHIGKTWAQLEWVPEPPELGKSPLTH | |
| | | | YTIFWTNAQNQSFSAILNASSRGFVLHGLEPA | |
| | | | SLYHIHLMAASQAGAINSTVLTLMTLTPEGS | |
| | | | ELH | |
| IL-2Rγ | P31785 | 628-735 | VVISVGSMGLIISLLCVYFWLERTMPRIPTLK | 9 |
| (TM and | | | NLEDLVTEYHGNFSAWSGVSKGLAESLQPD | |
| ICD) | | | YSERLCLVSEIPPKGGALGEGPGASPCNQHSP | |
| | | | YWAPPCYTLKPET | |

TABLE 15A
Chimeric Cytokine Receptors

| | | | | | STAT | Other |
|---|---|---|---|---|---|---|
| Name | ECD | TMD | Box1/2 | ICD | site(s) | site(s) |
| G2R-1 | G-CSFR | IL-2Rβ + γc | IL-2Rβ + | IL-2Rβ + | 5, 5 | Shc |
| | | | γc | γc | | |
| G2R-2 | G-CSFR | gp130 | gp130 | IL-2RB | 5, 5 | Shc |
| G2R-3 | G-CSFR | G-CSFR | G-CSFR | IL-2RB | 5, 5 | Shc |
| G21R-1 | G-CSFR | G-CSFR | G-CSFR | IL-21R | 3 | |
| G21R-2 | G-CSFR | G-CSFR | G-CSFR | IL-21R | 3 | un- |
| G12R-1 | G-CSFR | G-CSFR | G-CSFR | IL-12RB2 | 4 | known |

TABLE 15B
Chimeric Cytokine Receptors

| | | | | | STAT | Other |
|---|---|---|---|---|---|---|
| Name | ECD | TMD | Box1/2 | ICD | site(s) | site(s) |
| G21/2R-1 | G-CSFR | G-CSFR | G-CSFR | G-CSFR, | 3, 5, 5 | Shc |
| | | | | IL-2Rβ | | |
| G12/2R-1 | G-CSFR | G-CSFR | G-CSFR | STAT4 | 4, 5 | Shc |
| | | | | site from IL- | | |
| | | | | 12Rβ₂ replaces | | |
| | | | | STAT5 site | | |
| | | | | from IL- | | |
| | | | | 2Rβ | | |
| G21/12/ | G-CSFR | G-CSFR | G-CSFR | Contains | 3, 4, 5 | Shc |
| 2R-1 | | | | STAT3 site | | |
| | | | | from | | |
| | | | | G-CSFR ICD; | | |
| | | | | STAT4 site | | |
| | | | | from IL- | | |
| | | | | 12Rβ₂ replaces | | |
| | | | | STAT5 site | | |
| | | | | from IL- | | |
| | | | | 2Rβ | | |
| G27/2R-1 | G-CSFR | gp130 | gp130 | gp130 + | 3, 5, 5 | SHP-2, |
| | | | | IL-2Rβ | | Shc |
| G7R-1 | G-CSFR | G-CSFR | G-CSFR | IL-7Rα | 5 | PI3K |

TABLE 16

| | | | | | | | | |
|---|---|---|---|---|---|---|---|---|
| gp130 | IWPNVPDPS | n/a | Y759 | n/a | n/a | Y767 | n/a | n/a |
| | SEQ ID NO: 84) | | | | | | | |
| | (P40189) | | | | | | | |
| IL- | LKCNTPDPS | Y338 | n/a | Y392, | n/a | Y392, | Y392, | n/a |
| 2Rβ | SEQ ID NO: 85) | | | Y510 | | Y510 | Y510 | |
| | (P14784) | | | | | | | |
| IL- | KIWAVPSPE | n/a | n/a | n/a | n/a | Y510 | Y510 | n/a |
| 21R | SEQ ID NO: 86 | | | | | | | |
| | (Q9HBE5) | | | | | | | |
| IL- | CSREIPDPA | n/a | n/a | Y800 | Y800 | Y800 | n/a | n/a |
| 12Rβ₂ | SEQ ID NO: 87 | | | | | | | |
| | (Q99665) | | | | | | | |
| IL- | VWPSLPDHK | n/a | n/a | Y449 | n/a | n/a | n/a | Y449 |
| 7Rα | SEQ ID NO: 88 | | | | | | | |
| | (P16871) | | | | | | | |

TABLE 17
Signal Peptide. The signal peptide consists of one of the following:

| | | | SEQ |
|---|---|---|---|
| | | | ID |
| Domain | Uniprot ID | Polypeptide Sequence | NO. |
| G-CSFR (Signal | Q99062 | MARLGNCSLTWAALIILLLPGSLE | 11 |
| peptide) | | | |
| GM-CSFR-alpha | P15509 | MLLLVTSLLLCELPHPAFLLIP | 12 |
| (Signal peptide) | | | |
| | Genbank | | |
| Domain | Accession ID | Nucleic Acid Sequence | |
| G-CSFR (Signal | NP_000751 | ATGGCAAGGCTGGGAAACTGCAGCCTGACTTGGGCTGCCCTGATC | 13 |
| peptide, native | | ATCCTGCTGCTCCCCGGAAGTCTGGAG | |
| sequence) | | | |
| GM-CSFR-alpha | NP_034100 | ATGCTGCTGCTAGTGACCTCCCTGCTGCTCTGTGAGCTGCCTCACCC | 14 |
| (Signal peptide, | | GGCGTTCCTGCTGATTCCT | |
| native sequence) | | | |

TABLE 18
Wild-type G-CSFR extracellular domain (ECD): The G-CSFR ECD consists of
one of the following:

| | | | SEQ |
|---|---|---|---|
| | | | ID |
| Domain | Uniprot ID | Polypeptide Sequence | NO. |
| G-CSFR (ECD) | Q99062 | ECGHISVSAPIVHLGDPITASCIIKQNCSHLDPEPQILWRLGAELQPGG | 15 |
| | | RQQRLSDGTQESIITLPHLNHTQAFLSCCLNWGNSLQILDQVELRAGY | |
| | | PPAIPHNLSCLMNLTTSSLICQWEPGPETHLPTSFTLKSFKSRGNCQTQ | |
| | | GDSILDCVPKDGQSHCCIPRKHLLLYQNMGIWVQAENALGTSMSPQL | |
| | | CLDPMDVVKLEPPMLRTMDPSPEAAPPQAGCLQLCWEPWQPGLHI | |
| | | NQKCELRHKPQRGEASWALVGPLPLEALQYELCGLLPATAYTLQIRCIR | |
| | | WPLPGHWSDWSPSLELRTTERAPTVRLDTWWRQRQLDPRTVQLFW | |
| | | KPVPLEEDSGRIQGYVVSWRPSGQAGAILPLCNTTELSCTFHLPSEAQE | |
| | | VALVAYNSAGTSRPTPVVFSESRGPALTRLHAMARDPHSLWVGWEP | |
| | | PNPWPQGYVIEWGLGPPSASNSNKTWRMEQNGRATGFLLKENIRPF | |
| | | QLYEIIVTPLYQDTMGPSQHVYAYSQEMAPSHAPELHLKHIGKTWAQ | |
| | | LEWVPEPPELGKSPLTHYTIFWTNAQNQSFSAILNASSRGFVLHGLEP | |
| | | ASLYHIHLMAASQAGATNSTVLTLMTLTPEGSELH | |
| Domain | Genbank | | |
| | Accession ID | Nucleic Acid Sequence | |
| G-CSFR (ECD, | NP_000751 | GAGTGCGGGCACATCAGTGTCTCAGCCCCCATCGTCCACCTGGGG | 16 |
| native sequence) | | GATCCCATCACAGCCTCCTGCATCATCAAGCAGAACTGCAGCCATC | |
| | | TGGACCCGGAGCCACAGATTCTGTGGAGACTGGGAGCAGAGCTTC | |
| | | AGCCCGGGGGCAGGCAGCAGCGTCTGTCTGATGGGACCCAGGAA | |
| | | TCTATCATCACCCTGCCCCACCTCAACCACACTCAGGCCTTTCTCTCC | |
| | | TGCTGCCTGAACTGGGGCAACAGCCTGCAGATCCTGGACCAGGTT | |
| | | GAGCTGCGCGCAGGCTACCCTCCAGCCATACCCCACAACCTCTCCT | |
| | | GCCTCATGAACCTCACAACCAGCAGCCTCATCTGCCAGTGGGAGCC | |
| | | AGGACCTGAGACCCACCTACCCACCAGCTTCACTCTGAAGAGTTTC | |
| | | AAGAGCCGGGGCAACTGTCAGACCCAAGGGGACTCCATCCTGGAC | |
| | | TGCGTGCCCAAGGACGGGCAGAGCCACTGCTGCATCCCACGCAAA | |
| | | CACCTGCTGTTGTACCAGAATATGGGCATCTGGGTGCAGGCAGAG | |
| | | AATGCGCTGGGGACCAGCATGTCCCCACAACTGTGTCTTGATCCCA | |
| | | TGGATGTTGTGAAACTGGAGCCCCCCATGCTGCGGACCATGGACC | |
| | | CCAGCCCTGAAGCGGCCCCTCCCCAGGCAGGCTGCCTACAGCTGT | |
| | | GCTGGGAGCCATGGCAGCCAGGCCTGCACATAAATCAGAAGTGTG | |
| | | AGCTGCGCCACAAGCCGCAGCGTGGAGAAGCCAGCTGGGCACTG | |
| | | GTGGGCCCCCTCCCCTTGGAGGCCCTTCAGTATGAGCTCTGCGGGC | |
| | | TCCTCCCAGCCACGGCCTACACCCTGCAGATACGCTGCATCCGCTG | |
| | | GCCCCTGCCTGGCCACTGGAGCGACTGGAGCCCCAGCCTGGAGCT | |
| | | GAGAACTACCGAACGGGCCCCCACTGTCAGACTGGACACATGGTG | |
| | | GCGGCAGAGGCAGCTGGACCCCAGGACAGTGCAGCTGTTCTGGA | |
| | | AGCCAGTGCCCCTGGAGGAAGACAGCGGACGGATCCAAGGTTAT | |
| | | GTGGTTTCTTGGAGACCCTCAGGCCAGGCTGGGGCCATCCTGCCCC | |
| | | TCTGCAACACCACAGAGCTCAGCTGCACCTTCCACCTGCCTTCAGA | |
| | | AGCCCAGGAGGTGGCCCTTGTGGCCTATAACTCAGCCGGGACCTC | |
| | | TCGTCCCACTCCGGTGGTCTTCTCAGAAAGCAGAGGCCCAGCTCTG | |
| | | ACCAGACTCCATGCCATGGCCCGAGACCCTCACAGCCTCTGGGTAG | |
| | | GCTGGGAGCCCCCCAATCCATGGCCTCAGGGCTATGTGATTGAGT | |
| | | GGGGCCTGGGCCCCCCCAGCGCGAGCAATAGCAACAAGACCTGG | |
| | | AGGATGGAACAGAATGGGAGAGCCACGGGGTTTCTGCTGAAGGA | |
| | | GAACATCAGGCCCTTTCAGCTCTATGAGATCATCGTGACTCCCTTGT | |
| | | ACCAGGACACCATGGGACCCTCCCAGCATGTCTATGCCTACTCTCA | |
| | | AGAAATGGCTCCCTCCCATGCCCCAGAGCTGCATCTAAAGCACATT | |
| | | GGCAAGACCTGGGCACAGCTGGAGTGGGTGCCTGAGCCCCCTGA | |
| | | GCTGGGGAAGAGCCCCCTTACCCACTACACCATCTTCTGGACCAAC | |
| | | GCTCAGAACCAGTCCTTCTCCGCCATCCTGAATGCCTCCTCCCGTGG | |
| | | CTTTGTCCTCCATGGCCTGGAGCCCGCCAGTCTGTATCACATCCACC | |
| | | TCATGGCTGCCAGCCAGGCTGGGGCCACCAACAGTACAGTCCTCA | |
| | | CCCTGATGACCTTGACCCCAGAGGGGTCGGAGCTACAC | |
| G-CSFR (ECD, 4 | | GAGTGCGGGCACATCAGTGTCTCAGCCCCCATCGTCCACCTGGGG | 17 |
| codons altered) | | GATCCCATCACAGCCTCCTGCATCATCAAGCAGAACTGCAGCCATC | |
| | | TGGACCCGGAGCCACAGATTCTGTGGAGACTGGGAGCAGAGCTTC | |
| | | AGCCCGGGGGCAGGCAGCAGCGTCTGTCTGATGGGACCCAGGAA | |
| | | TCTATCATCACCCTGCCCCACCTCAACCACACTCAGGCCTTTCTCTCC | |
| | | TGCTGCCTGAACTGGGGCAACAGCCTGCAGATCCTGGACCAGGTT | |
| | | GAGCTGCGCGCAGGCTACCCTCCAGCCATACCCCACAACCTCTCCT | |
| | | GCCTCATGAACCTCACAACCAGCAGCCTCATCTGCCAGTGGGAGCC | |
| | | AGGACCTGAGACCCACCTACCCACCAGCTTCACTCTGAAGAGTTTC | |
| | | AAGAGCCGGGGCAACTGTCAGACCCAAGGGGACTCCATCCTGGAC | |
| | | TGCGTGCCCAAGGACGGGCAGAGCCACTGCTGCATCCCACGCAAA | |
| | | CACCTGCTGTTGTACCAGAATATGGGCATCTGGGTGCAGGCAGAG | |
| | | AATGCGCTGGGGACCAGCATGTCCCCACAACTGTGTCTTGATCCCA | |
| | | TGGATGTTGTGAAACTGGAGCCCCCCATGCTGCGGACCATGGACC | |
| | | CCAGCCCTGAAGCGGCCCCTCCCCAGGCAGGCTGCCTACAGCTGT | |
| | | GCTGGGAGCCATGGCAGCCAGGCCTGCACATAAATCAGAAGTGTG | |
| | | AGCTGCGCCACAAGCCGCAGCGTGGAGAAGCCAGCTGGGCACTG | |
| | | GTGGGCCCCCTCCCCTTGGAGGCCCTTCAGTATGAGCTCTGCGGGC | |
| | | TCCTCCCAGCCACGGCCTACACCCTGCAGATACGCTGCATCCGCTG | |
| | | GCCCCTGCCTGGCCACTGGAGCGACTGGAGCCCCAGCCTGGAGCT | |
| | | GAGAACTACCGAACGGGCCCCCACTGTCAGACTGGACACATGGTG | |
| | | GCGGCAGAGGCAGCTGGACCCCAGGACAGTGCAGCTGTTCTGGA | |
| | | AGCCAGTGCCCCTGGAGGAAGACAGCGGACGCATCCAAGGTTATG | |
| | | TGGTTTCTTGGAGACCCTCAGGCCAGGCTGGGGCCATCCTGCCCCT | |
| | | CTGCAACACCACAGAGCTCAGCTGCACCTTCCACCTGCCTTCAGAA | |
| | | GCCCAGGAGGTGGCCCTTGTGGCCTATAACTCAGCCGGGACCTCTC | |
| | | GCCCCACCCCGGTGGTCTTCTCAGAAAGCAGAGGCCCAGCTCTGAC | |
| | | CAGACTCCATGCCATGGCCCGAGACCCTCACAGCCTCTGGGTAGGC | |
| | | TGGGAGCCCCCCAATCCATGGCCTCAGGGCTATGTGATTGAGTGG | |
| | | GGCCTGGGCCCCCCCAGCGCGAGCAATAGCAACAAGACCTGGAG | |
| | | GATGGAACAGAATGGGAGAGCCACGGGGTTTCTGCTGAAGGAGA | |
| | | ACATCAGGCCCTTTCAGCTCTATGAGATCATCGTGACTCCCTTGTAC | |
| | | CAGGACACCATGGGACCCTCCCAGCATGTCTATGCCTACTCTCAAG | |
| | | AAATGGCTCCCTCCCATGCCCCAGAGCTGCATCTAAAGCACATTGG | |
| | | CAAGACCTGGGCACAGCTGGAGTGGGTGCCTGAGCCCCCTGAGCT | |
| | | GGGGAAGAGCCCCCTTACCCACTACACCATCTTCTGGACCAACGCT | |
| | | CAGAACCAGTCCTTCTCCGCCATCCTGAATGCATCCTCCCGTGGCTT | |
| | | TGTCCTCCATGGCCTGGAGCCCGCCAGTCTGTATCACATCCACCTCA | |
| | | TGGCTGCCAGCCAGGCTGGGGCCACCAACAGTACAGTCCTCACCCT | |
| | | GATGACCTTGACCCCAGAGGGGTCGGAGCTACAC | |

TABLE 19
Transmembrane domain (TM). The TM consists of one of the following:

| | | | SEQ |
|---|---|---|---|
| | | | ID |
| Domain | Uniprot ID | Polypeptide Sequence | NO. |
| G-CSFR (TM) | Q99062 | IILGLFGLLLLLTCLCGTAWLCC | 18 |
| gp130 (TM) | P40189 | AIVVPVCLAFLLTTLLGVLFCF | 19 |
| IL-2Rb (TM) | P14784 | IPWLGHLLVGLSGAFGFIILVYLLI | 20 |
| IL-2RG (TM) | P31785 | VVISVGSMGLIISLLCVYFWL | 21 |
| | Genbank | | |
| Domain | Accession ID | Nucleic Acid Sequence | |
| G-CSFR (TM) | NP_000751 | ATCATCCTGGGCCTGTTCGGCCTCCTGCTGTTGCTCAC | 22 |
| | | CTGCCTCTGTGGAACTGCCTGGCTCTGTTGC | |
| gp130 (TM) | NM_002184 | GCCATAGTCGTGCCTGTTTGCTTAGCATTCCTATTGAC | 23 |
| | | AACTCTTCTGGGAGTGCTGTTCTGCTTT | |
| IL-2Rb (TM) | NM_000878 | ATTCCGTGGCTCGGCCACCTCCTCGTGGGTCTCAGCGG | 24 |
| | | GGCTTTTGGCTTCATCATCTTAGTGTACTTGCTGATC | |
| IL-2RG (TM) | NP_000197 | GTGGTTATCTCTGTTGGCTCCATGGGATTGATTATCAG | 25 |
| | | CCTTCTCTGTGTGTATTTCTGGCTG | |

TABLE 20
Intracellular domain (ICD). The ICD consists of one of the following:

| | | | SEQ |
|---|---|---|---|
| | | | ID |
| Domain | Uniprot ID | Polypeptide Sequence | NO. |
| IL-2Rb (ICD, G2R- | P14784 | NCRNTGPWLKKVLKCNTPDPSKFFSQLSSEHGGDVQKWLSSPFPSSSF | 26 |
| 1) | | SPGGLAPEISPLEVLERDKVTQLLLQQDKVPEPASLSSNHSLTSCFTNQ | |
| | | GYFFFHLPDALEIEACQVYFTYDPYSEEDPDEGVAGAPTGSSPQPLQPL | |
| | | SGEDDAYCTFPSRDDLLLFSPSLLGGPSPPSTAPGGSGAGEERMPPSL | |
| | | QERVPRDWDPQPLGPPTPGVPDLVDFQPPPELVLREAGEEVPDAGP | |
| | | REGVSFPWSRPPGQGEFRALNARLPLNTDAYLSLQELQGQDPTHLV* | |
| IL-2RG (ICD, G2R- | P31785 | ERTMPRIPTLKNLEDLVTEYHGNFSAWSGVSKGLAESLQPDYSERLCL | 27 |
| 1) | | VSEIPPKGGALGEGPGASPCNQHSPYWAPPCYTLKPET* | |
| gp130 (ICD, G2R- | P40189 | NKRDLIKKHIWPNVPDPSKSHIAQWSPHTPPRHNFNSKDQMYSDGN | 28 |
| 2) | | FTDVSVVEIEANDKKPFPEDLKSLDLFKKEKINTEGHSSGIGGSSCMSSS | |
| | | RPSIS | |
| IL-2Rb (ICD, G2R- | P14784 | ASLSSNHSLTSCFTNQGYFFFHLPDALEIEACQVYFTYDPYSEEDPDEG | 29 |
| 2) | | VAGAPTGSSPQPLQPLSGEDDAYCTFPSRDDLLLFSPSLLGGPSPPSTA | |
| | | PGGSGAGEERMPPSLQERVPRDWDPQPLGPPTPGVPDLVDFQPPPE | |
| | | LVLREAGEEVPDAGPREGVSFPWSRPPGQGEFRALNARLPLNTDAYLS | |
| | | LQELQGQDPTHLV* | |
| G-CSFR (ICD, G2R- | Q99062 | SPNRKNPLWPSVPDPAHSSLGSWVPTIMEEDAFQLPGLGTPPITKLTV | 30 |
| 3) | | LEEDEKKPVPWESH | |
| IL-2Rb (ICD, G2R- | P14784 | SSNHSLTSCFTNQGYFFFHLPDALEIEACQVYFTYDPYSEEDPDEGVAG | 31 |
| 3) | | APTGSSPQPLQPLSGEDDAYCTFPSRDDLLLFSPSLLGGPSPPSTAPGG | |
| | | SGAGEERMPPSLQERVPRDWDPQPLGPPTPGVPDLVDFQPPPELVL | |
| | | REAGEEVPDAGPREGVSFPWSRPPGQGEFRALNARLPLNTDAYLSLQ | |
| | | ELQGQDPTHLV* | |
| G-CSFR (ICD, | Q99062 | SPNRKNPLWPSVPDPAHSSLGSWVPTIMEEDAFQLPGLGTPPITKLTV | 32 |
| G12R-1) | | LEEDEKKPVPWESHNSSET | |
| IL-12Rb2 (ICD, | Q99665 | AGDLPTHDGYLPSNIDDLPSHEAPLADSLEELEPQHISLSVFPSSSLHPL | 33 |
| G12R-1) | | TFSCGDKLTLDQLKMRCDSLML* | |
| G-CSFR (ICD, | Q99062 | SPNRKNPLWPSVPDPAHSSLGSWVPTIMEEDAFQLPGLGTPPITKLTV | 34 |
| G21R-1) | | LEEDEKKPVPWESHNSSET | |
| IL-21R (ICD, | Q9HBE5 | SPGDEGPPRSYLRQWVVIPPPLSSPGPQAS* | 35 |
| G21R-1) | | | |
| G-CSFR (ICD, | Q99062 | SPNRKNPLWPSVPDPAHSSLGSWVPTIMEEDAFQLPGLGTPPITKLTV | 36 |
| G21R-2) | | LEEDEKKPVPWESHN | |
| IL-21R (ICD, | Q9HBE5 | QNSGGSAYSEERDRPYGLVSIDTVTVLDAEGPCTWPCSCEDDGYPAL | 37 |
| G21R-2) | | DLDAGLEPSPGLEDPLLDAGTTVLSCGCVSAGSPGLGGPLGSLLDRLKP | |
| | | PLADGEDWAGGLPWGGRSPGGVSESEAGSPLAGLDMDTFDSGFVG | |
| | | SDCSSPVECDFTSPGDEGPPRSYLRQWVVIPPPLSSPGPQAS* | |
| G-CSFR (ICD, | Q99062 | SPNRKNPLWPSVPDPAHSSLGSWVPTIMEEDAFQLPGLGTPPITKLTV | 38 |
| G21/2R-1) | | LEEDEKKPVPWESHNSSETCGLPTLVQTYVLQGDPRAVSTQPQSQ | |
| IL-2Rb (ICD, | P14784 | SSNHSLTSCFTNQGYFFFHLPDALEIEACQVYFTYDPYSEEDPDEGVAG | 39 |
| G21/2R-1) | | APTGSSPQPLQPLSGEDDAYCTFPSRDDLLLFSPSLLGGPSPPSTAPGG | |
| | | SGAGEERMPPSLQERVPRDWDPQPLGPPTPGVPDLVDFQPPPELVL | |
| | | REAGEEVPDAGPREGVSFPWSRPPGQGEFRALNARLPLNTDAYLSLQ | |
| | | ELQGQDPTHLV* | |
| G-CSFR (ICD, | Q99062 | SPNRKNPLWPSVPDPAHSSLGSWVPTIMEEDAFQLPGLGTPPITKLTV | 40 |
| G12/2R-1) | | LEEDEKKPVPWESH | |
| IL-2Rb (ICD, | P14784 | SSNHSLTSCFTNQGYFFFHLPDALEIEACQVYFTYDPYSEEDP | 41 |
| G12/2R-1) | | | |
| IL-12Rb2 (ICD, | Q99665 | AGDLPTHDGYLPSNIDDLPS | 42 |
| G12/2R-1) | | | |
| IL-2Rb (ICD, | P14784 | GGPSPPSTAPGGSGAGEERMPPSLQERVPRDWDPQPLGPPTPGVPD | 43 |
| G12/2R-1) | | LVDFQPPPELVLREAGEEVPDAGPREGVSFPWSRPPGQGEFRALNAR | |
| | | LPLNTDAYLSLQELQGQDPTHLV* | |
| G-CSFR (ICD, | Q99062 | SPNRKNPLWPSVPDPAHSSLGSWVPTIMEEDAFQLPGLGTPPITKLTV | 44 |
| G21/12/2R-1) | | LEEDEKKPVPWESHNSSETCGLPTLVQTYVLQGDPRAVSTQPQSQ | |
| IL-2Rb (ICD, | P14784 | SSNHSLTSCFTNQGYFFFHLPDALEIEACQVYFTYDPYSEEDP | 45 |
| G21/12/2R-1) | | | |
| IL-12Rb2 (ICD, | Q99665 | AGDLPTHDGYLPSNIDDLPS | 46 |
| G21/12/2R-1) | | | |
| IL-2Rb (ICD, | P14784 | GGPSPPSTAPGGSGAGEERMPPSLQERVPRDWDPQPLGPPTPGVPD | 47 |
| G21/12/2R-1) | | LVDFQPPPELVLREAGEEVPDAGPREGVSFPWSRPPGQGEFRALNAR | |
| | | LPLNTDAYLSLQELQGQDPTHLV* | |
| gp130 (ICD, | P40189 | NKRDLIKKHIWPNVPDPSKSHIAQWSPHTPPRHNFNSKDQMYSDGN | 48 |
| G27/2R-1) | | FTDVSVVEIEANDKKPFPEDLKSLDLFKKEKINTEGHSSGIGGSSCMSSS | |
| | | RPSISSSDENESSQNTSSTVQYSTVVHSGYRHQVPSVQVFSR | |
| IL-2Rb (ICD, | P14784 | ASLSSNHSLTSCFTNQGYFFFHLPDALEIEACQVYFTYDPYSEEDPDEG | 49 |
| G27/2R-1) | | VAGAPTGSSPQPLQPLSGEDDAYCTFPSRDDLLLFSPSLLGGPSPPSTA | |
| | | PGGSGAGEERMPPSLQERVPRDWDPQPLGPPTPGVPDLVDFQPPPE | |
| | | LVLREAGEEVPDAGPREGVSFPWSRPPGQGEFRALNARLPLNTDAYLS | |
| | | LQELQGQDPTHLV* | |
| G-CSFR (ICD, G7R- | Q99062 | SPNRKNPLWPSVPDPAHSSLGSWVPTIMEEDAFQLPGLGTPPITKLTV | 50 |
| 1) | | LEEDEKKPVPWESH | |
| IL-7Ra (ICD, G7R- | P16871 | SGKNGPHVYQDLLLSLGTTNSTLPPPFSLQSGILTLNPVAQGQPILTSLG | 51 |
| 1) | | SNQEEAYVTMSSFYQNQ* | |
| G-CSFR (ICD, | Q99062 | SPNRKNPLWPSVPDPAHSSLGSWVPTIMEEDAFQLPGLGTPPITKLTV | 52 |
| G21/7R-1) | | LEEDEKKPVPWESHNSSETCGLPTLVQTYVLQGDPRAVSTQPQSQ | |
| IL7R (ICD, | P16871 | SSSRSLDCRESGKNGPHVYQDLLLSLGTTNSTLPPPFSLQSGILTLNPVA | 53 |
| G21/7R-1) | | QGQPILTSLGSNQEEAYVTMSSFYQNQ* | |
| | Genbank | Nucleic Acid Sequence | |
| Domain | Accession ID | | |
| IL-2RB (ICD, G2R- | NM_000878 | AACTGCAGGAACACCGGGCCATGGCTGAAGAAGGTCCTGAAGTGT | 54 |
| 1) | | AACACCCCAGACCCCTCGAAGTTCTTTTCCCAGCTGAGCTCAGAGC | |
| | | ATGGAGGAGACGTCCAGAAGTGGCTCTCTTCGCCCTTCCCCTCATC | |
| | | GTCCTTCAGCCCTGGCGGCCTGGCACCTGAGATCTCGCCACTAGAA | |
| | | GTGCTGGAGAGGGACAAGGTGACGCAGCTGCTCCTGCAGCAGGA | |
| | | CAAGGTGCCTGAGCCCGCATCCTTAAGCAGCAACCACTCGCTGACC | |
| | | AGCTGCTTCACCAACCAGGGTTACTTCTTCTTCCACCTCCCGGATGC | |
| | | CTTGGAGATAGAGGCCTGCCAGGTGTACTTTACTTACGACCCCTAC | |
| | | TCAGAGGAAGACCCTGATGAGGGTGTGGCCGGGGCACCCACAGG | |
| | | GTCTTCCCCCCAACCCCTGCAGCCTCTGTCAGGGGAGGACGACGCC | |
| | | TACTGCACCTTCCCCTCCAGGGATGACCTGCTGCTCTTCTCCCCCAG | |
| | | TCTCCTCGGTGGCCCCAGCCCCCCAAGCACTGCCCCTGGGGGCAGT | |
| | | GGGGCCGGTGAAGAGAGGATGCCCCCTTCTTTGCAAGAAAGAGTC | |
| | | CCCAGAGACTGGGACCCCCAGCCCCTGGGGCCTCCCACCCCAGGA | |
| | | GTCCCAGACCTGGTGGATTTTCAGCCACCCCCTGAGCTGGTGCTGC | |
| | | GAGAGGCTGGGGAGGAGGTCCCTGACGCTGGCCCCAGGGAGGGA | |
| | | GTCAGTTTCCCCTGGTCCAGGCCTCCTGGGCAGGGGGAGTTCAGG | |
| | | GCCCTTAATGCTCGCCTGCCCCTGAACACTGATGCCTACTTGTCCCT | |
| | | CCAAGAACTCCAGGGTCAGGACCCAACTCACTTGGTGTAG | |
| IL-2RG (ICD, G2R- | NP_000197 | GAACGGACGATGCCCCGAATTCCCACCCTGAAGAACCTAGAGGAT | 55 |
| 1) | | CTTGTTACTGAATACCACGGGAACTTTTCGGCCTGGAGTGGTGTGT | |
| | | CTAAGGGACTGGCTGAGAGTCTGCAGCCAGACTACAGTGAACGAC | |
| | | TCTGCCTCGTCAGTGAGATTCCCCCAAAAGGAGGGGCCCTTGGGG | |
| | | AGGGGCCTGGGGCCTCCCCATGCAACCAGCATAGCCCCTACTGGG | |
| | | CCCCCCCATGTTACACCCTAAAGCCTGAAACCTGA | |
| gp130 (ICD, G2R- | NM_002184 | AATAAGCGAGACCTAATTAAAAAACACATCTGGCCTAATGTTCCAG | 56 |
| 2) | | ATCCTTCAAAGAGTCATATTGCCCAGTGGTCACCTCACACTCCTCCA | |
| | | AGGCACAATTTTAATTCAAAAGATCAAATGTATTCAGATGGCAATT | |
| | | TCACTGATGTAAGTGTTGTGGAAATAGAAGCAAATGACAAAAAGC | |
| | | CTTTTCCAGAAGATCTGAAATCATTGGACCTGTTCAAAAAGGAAAA | |
| | | AATTAATACTGAAGGACACAGCAGTGGTATTGGGGGGTCTTCATG | |
| | | TATGTCATCTTCTAGGCCAAGCATTTCT | |
| IL-2Rb (ICD, G2R- | NM_000878 | GCATCCTTAAGCAGCAACCACTCGCTGACCAGCTGCTTCACCAACC | 57 |
| 2) | | AGGGTTACTTCTTCTTCCACCTCCCGGATGCCTTGGAGATAGAGGC | |
| | | CTGCCAGGTGTACTTTACTTACGACCCCTACTCAGAGGAAGACCCT | |
| | | GATGAGGGTGTGGCCGGGGCACCCACAGGGTCTTCCCCCCAACCC | |
| | | CTGCAGCCTCTGTCAGGGGAGGACGACGCCTACTGCACCTTCCCCT | |
| | | CCAGGGATGACCTGCTGCTCTTCTCCCCCAGTCTCCTCGGTGGCCCC | |
| | | AGCCCCCCAAGCACTGCCCCTGGGGGCAGTGGGGCCGGTGAAGA | |
| | | GAGGATGCCCCCTTCTTTGCAAGAAAGAGTCCCCAGAGACTGGGA | |
| | | CCCCCAGCCCCTGGGGCCTCCCACCCCAGGAGTCCCAGACCTGGTG | |
| | | GATTTTCAGCCACCCCCTGAGCTGGTGCTGCGAGAGGCTGGGGAG | |
| | | GAGGTCCCTGACGCTGGCCCCAGGGAGGGAGTCAGTTTCCCCTGG | |
| | | TCCAGGCCTCCTGGGCAGGGGGAGTTCAGGGCCCTTAATGCTCGC | |
| | | CTGCCCCTGAACACTGATGCCTACTTGTCCCTCCAAGAACTCCAGG | |
| | | GTCAGGACCCAACTCACTTGGTGTAG | |
| G-CSFR (ICD, G2R- | NP_000751 | AGCCCCAACAGGAAGAATCCCCTCTGGCCAAGTGTCCCAGACCCAG | 58 |
| 3) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCAT | |
| IL-2Rb (ICD, G2R- | NM_000878 | AGCAGCAACCACTCGCTGACCAGCTGCTTCACCAACCAGGGTTACT | 59 |
| 3) | | TCTTCTTCCACCTCCCGGATGCCTTGGAGATAGAGGCCTGCCAGGT | |
| | | GTACTTTACTTACGACCCCTACTCAGAGGAAGACCCTGATGAGGGT | |
| | | GTGGCCGGGGCACCCACAGGGTCTTCCCCCCAACCCCTGCAGCCTC | |
| | | TGTCAGGGGAGGACGACGCCTACTGCACCTTCCCCTCCAGGGATG | |
| | | ACCTGCTGCTCTTCTCCCCCAGTCTCCTCGGTGGCCCCAGCCCCCCA | |
| | | AGCACTGCCCCTGGGGGCAGTGGGGCCGGTGAAGAGAGGATGCC | |
| | | CCCTTCTTTGCAAGAAAGAGTCCCCAGAGACTGGGACCCCCAGCCC | |
| | | CTGGGGCCTCCCACCCCAGGAGTCCCAGACCTGGTGGATTTTCAGC | |
| | | CACCCCCTGAGCTGGTGCTGCGAGAGGCTGGGGAGGAGGTCCCTG | |
| | | ACGCTGGCCCCAGGGAGGGAGTCAGTTTCCCCTGGTCCAGGCCTC | |
| | | CTGGGCAGGGGGAGTTCAGGGCCCTTAATGCTCGCCTGCCCCTGA | |
| | | ACACTGATGCCTACTTGTCCCTCCAAGAACTCCAGGGTCAGGACCC | |
| | | AACTCACTTGGTGTAG | |
| G-CSFR (ICD, | NP_000751 | AGCCCCAACAGGAAGAATCCCCTCTGGCCAAGTGTCCCAGACCCAG | 60 |
| G12R-1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCATAACAGCTCAGAGACC | |
| IL-12Rb2 (ICD, | NP_001550 | GCAGGTGACCTTCCCACCCATGATGGCTACTTACCCTCCAACATAG | 61 |
| G12R-1) | | ATGACCTCCCCTCACATGAGGCACCTCTCGCTGACTCTCTGGAAGA | |
| | | ACTGGAGCCTCAGCACATCTCCCTTTCTGTTTTCCCCTCAAGTTCTCT | |
| | | TCACCCACTCACCTTCTCCTGTGGTGATAAGCTGACTCTGGATCAGT | |
| | | TAAAGATGAGGTGTGACTCCCTCATGCTCTGA | |
| G-CSFR (ICD, | NP_000751 | AGCCCCAACAGGAAGAATCCCCTCTGGCCAAGTGTCCCAGACCCAG | 62 |
| G21R-1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCATAACAGCTCAGAGACC | |
| IL-21R (ICD, | NP_068570 | AGCCCTGGGGACGAAGGACCCCCCCGGAGCTACCTCCGCCAGTGG | 63 |
| G21R-1) | (1codon | GTGGTCATTCCTCCGCCACTTTCGAGCCCTGGACCCCAGGCCAGCT | |
| | altered) | AA | |
| G-CSFR (ICD, | NP_000751 | AGCCCCAACAGGAAGAATCCCCTCTGGCCAAGTGTCCCAGACCCAG | 64 |
| G21R-2) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCATAAC | |
| IL-21R (ICD, | NP_068570 | CAGAACTCGGGGGGCTCAGCTTACAGTGAGGAGAGGGATCGGCC | 65 |
| G21R-2) | (1codon | ATACGGCCTGGTGTCCATTGACACAGTGACTGTGCTAGATGCAGA | |
| | altered) | GGGGCCATGCACCTGGCCCTGCAGCTGTGAGGATGACGGCTACCC | |
| | | AGCCCTGGACCTGGATGCTGGCCTGGAGCCCAGCCCAGGCCTAGA | |
| | | GGACCCACTCTTGGATGCAGGGACCACAGTCCTGTCCTGTGGCTGT | |
| | | GTCTCAGCTGGCAGCCCTGGGCTAGGAGGGCCCCTGGGAAGCCTC | |
| | | CTGGACAGACTAAAGCCACCCCTTGCAGATGGGGAGGACTGGGCT | |
| | | GGGGGACTGCCCTGGGGTGGCCGGTCACCTGGAGGGGTCTCAGA | |
| | | GAGTGAGGCGGGCTCACCCCTGGCCGGCCTGGATATGGACACGTT | |
| | | TGACAGTGGCTTTGTGGGCTCTGACTGCAGCAGCCCTGTGGAGTGT | |
| | | GACTTCACCAGCCCTGGGGACGAAGGACCCCCCCGGAGCTACCTCC | |
| | | GCCAGTGGGTGGTCATTCCTCCGCCACTTTCGAGCCCTGGACCCCA | |
| | | GGCCAGC | |
| G-CSFR (ICD, | NP_000751 | AGCCCCAACAGGAAGAATCCCCTCTGGCCAAGTGTCCCAGACCCAG | 66 |
| G21/2R-1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCATAACAGCTCAGAGACCTGTGGCCTCCCCACTCTGGTCCAGAC | |
| | | CTATGTGCTCCAGGGGGACCCAAGAGCAGTTTCCACCCAGCCCCAA | |
| | | TCCCAG | |
| IL-2Rb (ICD, | NM_000878 | AGCAGCAACCACTCGCTGACCAGCTGCTTCACCAACCAGGGTTACT | 67 |
| G21/2R-1) | | TCTTCTTCCACCTCCCGGATGCCTTGGAGATAGAGGCCTGCCAGGT | |
| | | GTACTTTACTTACGACCCCTACTCAGAGGAAGACCCTGATGAGGGT | |
| | | GTGGCCGGGGCACCCACAGGGTCTTCCCCCCAACCCCTGCAGCCTC | |
| | | TGTCAGGGGAGGACGACGCCTACTGCACCTTCCCCTCCAGGGATG | |
| | | ACCTGCTGCTCTTCTCCCCCAGTCTCCTCGGTGGCCCCAGCCCCCCA | |
| | | AGCACTGCCCCTGGGGGCAGTGGGGCCGGTGAAGAGAGGATGCC | |
| | | CCCTTCTTTGCAAGAAAGAGTCCCCAGAGACTGGGACCCCCAGCCC | |
| | | CTGGGGCCTCCCACCCCAGGAGTCCCAGACCTGGTGGATTTTCAGC | |
| | | CACCCCCTGAGCTGGTGCTGCGAGAGGCTGGGGAGGAGGTCCCTG | |
| | | ACGCTGGCCCCAGGGAGGGAGTCAGTTTCCCCTGGTCCAGGCCTC | |
| | | CTGGGCAGGGGGAGTTCAGGGCCCTTAATGCTCGCCTGCCCCTGA | |
| | | ACACTGATGCCTACTTGTCCCTCCAAGAACTCCAGGGTCAGGACCC | |
| | | AACTCACTTGGTGTAG | |
| G-CSFR (ICD, | NP_000751 | AGCCCCAACAGGAAGAATCCCCTCTGGCCAAGTGTCCCAGACCCAG | 68 |
| G12/2R-1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCAT | |
| IL-2Rb (ICD, | NM_000878 | AGCAGCAACCACTCGCTGACCAGCTGCTTCACCAACCAGGGTTACT | 69 |
| G12/2R-1) | | TCTTCTTCCACCTCCCGGATGCCTTGGAGATAGAGGCCTGCCAGGT | |
| | | GTACTTTACTTACGACCCCTACTCAGAGGAAGACCCT | |
| IL-12Rb2 (ICD, | NP_001550 | GCAGGTGACCTTCCCACCCATGATGGCTACTTACCCTCCAACATAG | 70 |
| G12/2R-1) | | ATGACCTCCCCTCA | |
| IL-2Rb (ICD, | NM_000878 | GGTGGCCCCAGCCCCCCAAGCACTGCCCCTGGGGGCAGTGGGGCC | 71 |
| G12/2R-1) | | GGTGAAGAGAGGATGCCCCCTTCTTTGCAAGAAAGAGTCCCCAGA | |
| | | GACTGGGACCCCCAGCCCCTGGGGCCTCCCACCCCAGGAGTCCCA | |
| | | GACCTGGTGGATTTTCAGCCACCCCCTGAGCTGGTGCTGCGAGAG | |
| | | GCTGGGGAGGAGGTCCCTGACGCTGGCCCCAGGGAGGGAGTCAG | |
| | | TTTCCCCTGGTCCAGGCCTCCTGGGCAGGGGGAGTTCAGGGCCCTT | |
| | | AATGCTCGCCTGCCCCTGAACACTGATGCCTACTTGTCCCTCCAAGA | |
| | | ACTCCAGGGTCAGGACCCAACTCACTTGGTGTAG | |
| G-CSFR (ICD, | NP_000751 | AGCCCCAACAGGAAGAATCCCCTCTGGCCAAGTGTCCCAGACCCAG | 72 |
| G21/12/2R-1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCATAACAGCTCAGAGACCTGTGGCCTCCCCACTCTGGTCCAGAC | |
| | | CTATGTGCTCCAGGGGGACCCAAGAGCAGTTTCCACCCAGCCCCAA | |
| | | TCCCAG | |
| IL-2Rb (ICD, | NM_000878 | AGCAGCAACCACTCGCTGACCAGCTGCTTCACCAACCAGGGTTACT | 73 |
| G21/12/2R-1) | | TCTTCTTCCACCTCCCGGATGCCTTGGAGATAGAGGCCTGCCAGGT | |
| | | GTACTTTACTTACGACCCCTACTCAGAGGAAGACCCT | |
| IL-12Rb2 (ICD, | NP_001550 | GCAGGTGACCTTCCCACCCATGATGGCTACTTACCCTCCAACATAG | 74 |
| G21/12/2R-1) | | ATGACCTCCCCTCA | |
| IL-2Rb (ICD, | NM_000878 | GGTGGCCCCAGCCCCCCAAGCACTGCCCCTGGGGGCAGTGGGGCC | 75 |
| G21/12/2R-1) | | GGTGAAGAGAGGATGCCCCCTTCTTTGCAAGAAAGAGTCCCCAGA | |
| | | GACTGGGACCCCCAGCCCCTGGGGCCTCCCACCCCAGGAGTCCCA | |
| | | GACCTGGTGGATTTTCAGCCACCCCCTGAGCTGGTGCTGCGAGAG | |
| | | GCTGGGGAGGAGGTCCCTGACGCTGGCCCCAGGGAGGGAGTCAG | |
| | | TTTCCCCTGGTCCAGGCCTCCTGGGCAGGGGGAGTTCAGGGCCCTT | |
| | | AATGCTCGCCTGCCCCTGAACACTGATGCCTACTTGTCCCTCCAAGA | |
| | | ACTCCAGGGTCAGGACCCAACTCACTTGGTGTAG | |
| gp130 (ICD, | NM_002184 | AATAAGCGAGACCTAATTAAAAAACACATCTGGCCTAATGTTCCAG | 76 |
| G27/2R-1) | (3 codons | ATCCTTCAAAGAGTCATATTGCCCAGTGGTCACCTCACACTCCTCCA | |
| | altered) | AGGCACAATTTCAATTCAAAGGATCAAATGTATTCAGATGGCAATT | |
| | | TCACTGATGTAAGTGTTGTGGAAATAGAAGCAAATGACAAAAAGC | |
| | | CTTTTCCAGAAGATCTGAAATCATTGGACCTGTTCAAAAAGGAAAA | |
| | | AATTAATACTGAAGGACACAGCAGTGGTATTGGGGGGTCTTCATG | |
| | | TATGTCATCTTCTAGGCCAAGCATTTCTAGCAGTGATGAAAATGAA | |
| | | TCTTCACAAAACACTTCGAGCACTGTCCAGTATTCTACCGTGGTACA | |
| | | CAGTGGCTACAGACACCAAGTTCCGTCAGTCCAAGTCTTCTCAAGA | |
| IL-2Rb (ICD, | NM_000878 | GCATCCTTAAGCAGCAACCACTCGCTGACCAGCTGCTTCACCAACC | 77 |
| G27/2R-1) | (2 codons | AGGGTTACTTCTTCTTCCACCTCCCGGATGCCTTGGAGATAGAGGC | |
| | altered) | CTGCCAGGTGTACTTTACTTACGACCCCTACTCAGAGGAAGACCCT | |
| | | GATGAGGGTGTGGCCGGGGCACCCACAGGGTCTTCCCCCCAACCC | |
| | | CTGCAGCCTCTGTCAGGGGAGGACGACGCCTACTGCACCTTCCCCT | |
| | | CCAGGGATGACCTGCTGCTCTTCTCCCCCAGTCTCCTCGGTGGCCCC | |
| | | AGCCCCCCAAGCACTGCCCCTGGGGGCAGTGGGGCCGGTGAAGA | |
| | | GAGGATGCCCCCTTCTTTGCAAGAAAGAGTCCCCAGAGACTGGGA | |
| | | CCCCCAGCCCCTGGGGCCTCCCACCCCAGGAGTCCCAGACCTGGTG | |
| | | GATTTTCAGCCACCCCCTGAGCTGGTGCTGCGAGAGGCTGGGGAG | |
| | | GAGGTCCCTGACGCTGGCCCCAGGGAGGGAGTCAGTTTCCCCTGG | |
| | | TCCAGGCCTCCTGGGCAGGGGGAGTTCAGGGCCCTTAATGCTCGC | |
| | | CTGCCCCTGAACACTGATGCCTACTTGTCCCTCCAAGAACTCCAGG | |
| | | GTCAGGACCCAACTCACTTGGTGTAG | |
| G-CSFR (ICD, G7R- | NP_000751 | AGCCCCAACAGGAAGAATCCCCTCTGGCCAAGTGTCCCAGACCCAG | 78 |
| 1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCAT | |
| IL-7Ra (ICD, G7R- | NP_002176 | AGTGGCAAGAATGGGCCTCATGTGTACCAGGACCTCCTGCTTAGCC | 79 |
| 1) | | TTGGGACTACAAACAGCACGCTGCCCCCTCCATTTTCTCTCCAATCT | |
| | | GGAATCCTGACATTGAACCCAGTTGCTCAGGGTCAGCCCATTCTTA | |
| | | CTTCCCTGGGATCAAATCAAGAAGAAGCATATGTCACCATGTCCAG | |
| | | CTTCTACCAAAACCAGTGA | |
| G-CSFR (ICD, | NP_000751 | AGCCCCAACAGGAAGAATCCCCTCTGGCCAAGTGTCCCAGACCCAG | 80 |
| G21/7R-1) | | CTCACAGCAGCCTGGGCTCCTGGGTGCCCACAATCATGGAGGAGG | |
| | | ATGCCTTCCAGCTGCCCGGCCTTGGCACGCCACCCATCACCAAGCT | |
| | | CACAGTGCTGGAGGAGGATGAAAAGAAGCCGGTGCCCTGGGAGT | |
| | | CCCATAACAGCTCAGAGACCTGTGGCCTCCCCACTCTGGTCCAGAC | |
| | | CTATGTGCTCCAGGGGGACCCAAGAGCAGTTTCCACCCAGCCCCAA | |
| | | TCCCAG | |
| IL7R (ICD, | NP_002176 | TCCTCTTCCAGGTCCCTAGACTGCAGGGAGAGTGGCAAGAATGGG | 81 |
| G21/7R-1) | | CCTCATGTGTACCAGGACCTCCTGCTTAGCCTTGGGACTACAAACA | |
| | | GCACGCTGCCCCCTCCATTTTCTCTCCAATCTGGAATCCTGACATTG | |
| | | AACCCAGTTGCTCAGGGTCAGCCCATTCTTACTTCCCTGGGATCAA | |
| | | ATCAAGAAGAAGCATATGTCACCATGTCCAGCTTCTACCAAAACCA | |
| | | GTGA | |

1. A receptor, comprising:
a variant extracellular domain (ECD) of Granulocyte Colony-Stimulating Factor Receptor (G-CSFR), wherein
the variant ECD of G-CSFR comprises at least one mutation in a site II interface region, at least one mutation in a site III interface region, or combinations thereof.

2. The receptor of claim 1, wherein
the at least one mutation in the site II interface region is located at an amino acid position of the G-CSFR ECD selected from the group consisting of amino acid position 141, 167, 168, 171, 172, 173, 174, 197, 199, 200, 202 and 288 of SEQ ID NO. 2.

3. The receptor of claim 2, wherein the at least one mutation in the site II interface region is selected from the group of mutations of the G-CSFR ECD consisting of R141E, R167D, K168D, K168E, L171E, L172E, Y173K, Q174E, D197K, D197R, M199D, D200K, D200R, V202D, R288D, and R288E.

4. The receptor of any one of the above claims, wherein
the at least one mutation in the site III interface region is selected from the group of mutations of the G-CSFR ECD selected from the group consisting of amino acid position 30, 41, 73, 75, 79, 86, 87, 88, 89, 91, and 93 of SEQ ID NO. 2.

5. The receptor of claim 4, wherein
the at least one mutation in the site III interface region is selected from the group of mutations of the G-CSFR ECD consisting of S30D, R41E, Q73W, F75KF, S79D, L86D, Q87D, I88E, L89A, Q91D, Q91K, and E93K.

6. The receptor of any one of the above claims, wherein the G-CSFR ECD comprises a combination of mutations of a design number in Table 6; wherein the mutations correspond to an amino acid position of SEQ ID NO. 2.

7. The receptor of any one of the above claims, wherein
the G-CSFR ECD comprises the mutations: R41E, R141E, and R167D.

8. The receptor of any one of the above claims, wherein
the receptor is a chimeric receptor.

9. The receptor of any one of the above claims, wherein
the receptor is expressed on a cell.

10. The receptor of claim 9, wherein the cell is an immune cell and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

11. The receptor of any one of the above claims, wherein
activation of the receptor by a variant G-CSF causes a cellular response selected from the group consisting of proliferation, viability and enhanced activity of a cell expressing the receptor.

12. A nucleic acid encoding the receptor of any one of claims 1-11.

13. An expression vector comprising the nucleic acid of claim 12.

14. A cell engineered to express the receptor of any one of claims 1-11.

15. The cell of claim 14, wherein the cell is a T cell or NK cell.

16. A variant Granulocyte Colony-Stimulating Factor (G-CSF), wherein
the variant G-CSF comprises at least one mutation in a site II interface region, at least one mutation in a site III interface region, or combinations thereof.

17. The variant G-CSF of claim 16, wherein the at least one mutation in the site II interface region is located at an amino acid position selected from the group consisting of amino acid position 12, 16, 19, 20, 104, 108, 109, 112, 115, 116, 118, 119, 122 and 123 of SEQ ID NO. 1.

18. The variant G-CSF of claim 17, wherein
the at least one mutation in the site II interface region is selected from the group of mutations consisting of: S12E, S12K, S12R, K16D, L18F, E19K, Q20E, D104K, D104R, L108K, L108R, D109R, D112R, D112K, T115E, T115K, T116D, Q119E, Q119R, E122K, E122R, and E123R.

19. The variant G-CSF of any one of claims 16-18, wherein
the at least one mutation in the site III interface region is selected from the group of mutations selected from the group consisting of amino acid position 38, 39, 40, 41, 46, 47, 48, 49, and 147 of SEQ ID NO. 1.

20. The variant G-CSF of claim 19, wherein
the at least one mutation in the site III interface region is selected from the group of mutations consisting of: T38R, Y39E, K40D, K40F, L41D, L41E, L41K, E46R, L47D, V48K, V48R, L49K, and R147E.

21. The variant G-CSF of any one of claims 16-20, wherein
the variant G-CSF comprises a combination of mutations of a design number in Table 6;
wherein the mutations correspond to an amino acid position of SEQ ID NO. 1.

22. The variant G-CSF of claim 21, wherein
the variant G-CSF comprises the mutations: E46R, L108K and D112R.

23. The variant G-CSF of any one of claims 16-22, wherein
the variant G-CSF binds selectively to a receptor of any one of claims 1-11.

24. The variant G-CSF of claim 23, wherein the receptor is expressed on a cell.

25. The variant G-CSF of claim 24, wherein the cell is an immune cell.

26. The variant G-CSF of claim 25, wherein the immune cell is
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

27. The variant G-CSF of claim 26, wherein
the selective binding of the variant G-CSF to the receptor causes a cellular response selected from the group consisting of proliferation, viability and enhanced activity of the T cell or NK cell.

28. A nucleic acid encoding the variant G-CSF of any one of claims 16-27.

29. An expression vector comprising the nucleic acid of claim 28.

30. A cell engineered to express the variant G-CSF of any one of claims 16-27.

31. The cell of claim 30, wherein the cell is an immune cell.

32. A system for selective activation of a receptor expressed on a cell surface, the system comprising:
(a) the receptor of any one of claims 1-22; and
(b) the variant G-CSF of any one of claims 16-27; wherein
the receptor comprises at least one mutation in a site II interface region, a site III interface region, or combinations thereof, and the variant G-CSF comprises at least one mutation in an amino acid sequence of G-CSF that binds the site II interface region of the receptor, the site III interface region of the receptor, or combinations thereof; and wherein
the variant G-CSF binds the receptor preferentially over a wild type G-CSFR ECD, and the receptor binds the variant G-CSF preferentially over a wild type G-CSF.

33. The system of claim 32, wherein the receptor and the variant G-CSF comprise a combination of mutations of a site II interface of a design number of Table 2; wherein the receptor mutations correspond to an amino acid position of SEQ ID NO. 2 and the variant G-CSF mutations correspond to an amino acid position of SEQ ID NO. 1.

34. The system of claim 32, wherein the receptor and the variant G-CSF comprise a combination of mutations of a site III interface of a design number of Table 4; wherein the receptor mutations correspond to an amino acid position of SEQ ID NO. 2 and the variant G-CSF mutations correspond to an amino acid position of SEQ ID NO. 1.

35. The system of claim 32, wherein the receptor and the variant G-CSF comprise a combination of mutations of a site II interface, and a site III interface of a design number of Table 6; wherein the receptor mutations correspond to an amino acid position of SEQ ID NO. 2 and the variant G-CSF mutations correspond to an amino acid position of SEQ ID NO. 1.

36. The system of claim 35, wherein the combination of mutations comprises the mutations of design number 106; wherein the variant G-CSF comprises the E46R and D104K mutations corresponding to an amino acid position of SEQ ID NO. 1; and wherein the receptor comprises the R41E and K168D mutations corresponding to an amino acid position of SEQ ID NO. 2.

37. The system of claim 35, wherein the combination of mutations comprises the mutations of design number 117; wherein the variant G-CSF comprises the E46R, E122R, and E123R mutations corresponding to an amino acid position of SEQ ID NO. 1; and wherein the receptor comprises the R41E and R141E mutations corresponding to an amino acid position of SEQ ID NO. 2.

38. The system of claim 35, wherein the combination of mutations comprises the mutations of design number 130; wherein the variant G-CSF comprises the E46R, L108K, and D112R mutations corresponding to an amino acid position of SEQ ID NO. 1; and wherein the receptor comprises the R41E and R167D mutations corresponding to an amino acid position of SEQ ID NO. 2.

39. The system of claim 35, wherein the combination of mutations comprises the mutations of design number 134; wherein the variant G-CSF comprises the E46R, L108K, D112R, E122R, and E123R mutations corresponding to an amino acid position of SEQ ID NO. 1; and wherein the receptor comprises the R41E, R141E, and R167D mutations corresponding to an amino acid position of SEQ ID NO. 2.

40. The system of claim 35, wherein the combination of mutations comprises the mutations of design number 135; wherein the variant G-CSF comprises the E46R, T115K, E122R, and E123R mutations corresponding to an amino acid position of SEQ ID NO. 1; and wherein the receptor comprises the R41E, R141E, L171E, and Q174E mutations corresponding to an amino acid position of SEQ ID NO. 2.

41. The system of claim 35, wherein the combination of mutations comprises the mutations of design number 137; wherein the variant G-CSF comprises the E46R, L108K, and D112R mutations corresponding to an amino acid position of SEQ ID NO. 1; and wherein the receptor comprises the R41E, R141E, and R167D mutations corresponding to an amino acid position of SEQ ID NO. 2.

42. The system of claim 35, wherein the combination of mutations comprises the mutations of design number 300; wherein the variant G-CSF comprises the K40D, L41D, L108K, and D112R mutations corresponding to an amino acid position of SEQ ID NO. 1; and wherein the receptor comprises the F75K, Q91K and R167D mutations corresponding to an amino acid position of SEQ ID NO. 2.

43. The system of claim 35, wherein the combination of mutations comprises the mutations of design number 301; wherein the variant G-CSF comprises the T38R, E46R, L108K, and D112R mutations corresponding to an amino acid position of SEQ ID NO. 1; and wherein the receptor comprises the R41E, Q73E, and R167D mutations corresponding to an amino acid position of SEQ ID NO. 2.

44. The system of claim 35, wherein the combination of mutations comprises the mutations of design number 302; wherein the variant G-CSF comprises the E46R, L108K, and D112R mutations corresponding to an amino acid position of SEQ ID NO. 1; and wherein the receptor comprises the R41E, L86D, and R167D mutations corresponding to an amino acid position of SEQ ID NO. 2.

45. The system of claim 35, wherein the combination of mutations comprises the mutations of design number 303; wherein the variant G-CSF comprises the L108K, D112R, and R147E mutations corresponding to an amino acid position of SEQ ID NO. 1; and wherein the receptor comprises the E93K and R167D mutations corresponding to an amino acid position of SEQ ID NO. 2.

46. The system of claim 35, wherein the combination of mutations comprises the mutations of design number 304; wherein the variant G-CSF comprises the E46R, L108K, D112R, and R147E mutations corresponding to an amino acid position of SEQ ID NO. 1; and wherein the receptor comprises the R41E, E93K, and R167D mutations corresponding to an amino acid position of SEQ ID NO. 2.

47. The system of claim 35, wherein the combination of comprises the mutations of design number 305; wherein the variant G-CSF comprises the E19K, E46R, L108K, and D112R mutations corresponding to an amino acid position of SEQ ID NO. 1; and wherein the receptor comprises the R41E, R167D, and R288E mutations corresponding to an amino acid position of SEQ ID NO. 2.

48. The system of claim 35, wherein the combination of mutations comprises the mutations of design number 307; wherein the variant G-CSF comprises the S12E, K16D, E19K, and E46R mutations corresponding to an amino acid position of SEQ ID NO. 1; and wherein the receptor comprises the R41E, D197K, D200K and R288E mutations corresponding to an amino acid position of SEQ ID NO. 2.

49. The system of claim 35, wherein the combination of mutations comprises the mutations of design number 308; wherein the variant G-CSF comprises the E19R, E46R, D112K mutations corresponding to an amino acid position of SEQ ID NO. 1; and wherein the receptor comprises the R41E, R167D, V202D and R288E mutations corresponding to an amino acid position of SEQ ID NO. 2.

50. The system of claim 35, wherein the combination of mutations comprises the mutations of design number 400; wherein the variant G-CSF comprises the E19K, E46R, D109R, and D112R mutations corresponding to an amino acid position of SEQ ID NO. 1; and wherein the receptor comprises the R41E, R167D, M199D and R288D mutations corresponding to an amino acid position of SEQ ID NO. 2.

51. The system of claim 35, wherein the combination of mutations comprises the mutations of design number 401; wherein the variant G-CSF comprises the E19K, E46R, L108K, and D112R mutations corresponding to an amino acid position of SEQ ID NO. 1; and wherein the receptor comprises the R41E, R167D, and R288D mutations corresponding to an amino acid position of SEQ ID NO. 2.

52. The system of claim 35, wherein the combination of mutations comprises the mutations of design number 402; wherein the variant G-CSF comprises the E19K, E46R, D112K, and T115K mutations corresponding to an amino acid position of SEQ ID NO. 1; and wherein the receptor comprises the R41E, R167E, Q174E and R288E mutations corresponding to an amino acid position of SEQ ID NO. 2.

53. The system of claim 35, wherein the combination of mutations comprises the mutations of design number 403; wherein the variant G-CSF comprises the E19R, E46R, and D112K, mutations corresponding to an amino acid position of SEQ ID NO. 1; and wherein the receptor comprises the R41E, R167D, and R288E mutations corresponding to an amino acid position of SEQ ID NO. 2.

54. A method of selective activation of a receptor expressed on the surface of a cell, comprising:
contacting a receptor of any one of claims 1-11 with a variant G-CSF of claims 16-27.
The method of claim 54, wherein the receptor is expressed on an immune cell, and, optionally,
a T cell, and, optionally,
a NK cell, and, optionally,
a NKT cell, and, optionally,
a B cell, and, optionally,
a plasma cell, and, optionally,
a macrophage, and, optionally,
a dendritic cell, and, optionally,
the cell is a stem cell, and, optionally,
the cell is a primary cell, and, optionally,
the cell is a human cell.

55. The method of claim 54, wherein the selective activation of the immune cell causes a cellular response selected from the group consisting of proliferation, viability and enhanced activity of the immune cell.

56. A method of producing an immune cell expressing a receptor of any one of claims 1-11, comprising introducing to the cells the nucleic acid of claim 12 or the expression vector of claim 13.

57. A method of treating a subject in need thereof, comprising:
infusing into the subject the cell of claim 14.

58. The method of claim 57, further comprising administering a variant G-CSF of any one of claims 16-27 to the subject.

59. The method of claim 57 or 58, wherein the method is used to treat cancer.

60. The method of claim 57 or 58, wherein the method is used to treat an inflammatory condition.

61. The method of claim 57 or 58, wherein the method is used to treat graft rejection.

62. The method of claim 57 or 58, wherein the method is used to treat an infectious disease.

63. The method of claim 57 or 58; further comprising administering at least one additional active agent; optionally wherein the additional active agent is an additional cytokine.

64. A method of treating a subject in need thereof, wherein the method comprises:
i) isolating an immune cell-containing sample; (ii) transducing or transfecting the immune cells with a nucleic acid sequence encoding a variant cytokine receptor of claims 1-11; (iii) administering or infusing the immune cells from (ii) to the subject; and (iv) contacting the immune cells with a variant G-CSF of claims 16-27 that binds the variant receptor.

65. The method of claim 64, wherein the subject has undergone an immuno-depletion treatment prior to administering or infusing the cells to the subject.

66. The method of claim 64, wherein the immune cell-containing sample is isolated from the subject that will be administered or infused with the cells.

67. The method of claim 64, wherein the immune cells are contacted with the cytokine in vitro prior to administering or infusing the cells to the subject.

68. The method of claim 64, wherein the immune cells are contacted with the cytokine that binds the chimeric receptor for a sufficient time to activate signaling from the chimeric receptor.

69. A kit for treating a subject in need thereof, comprising: cells encoding a variant receptor of any one of claim 1-11 and instructions for use; optionally wherein the kit comprises a variant G-CSF of claims 16-27 that binds the variant receptor; and optionally wherein the cells are immune cells.

70. A kit for producing a system for selective activation of a receptor expressed on a cell surface, the kit comprising:
(a) the nucleic acid of claim 12 or the expression vector of claim 13;
(b) the variant G-CSF of any one of claims 16-27, the nucleic acid of claim 12 or the expression vector of claim 29; and
(c) instructions for use.

71. A kit for producing a chimeric receptor expressed on a cell, comprising:
cells comprising an expression vector encoding the variant receptor of any one of claims 1-11 and instructions for use; optionally wherein the cells are bacterial cells; and optionally wherein the kit comprises a variant G-CSF that binds the variant receptor.
